(19) United States
(12) Patent Application Publication
(10) Pub. No.: US 2006/0281110 A1
(43) Pub. Date: Dec. 14, 2006

(54) Methods to identify antimicrobial compounds that interrupt ribosome biogenesis

(75) Inventors: Robert A. Britton, East Lansing, MI (US); William C. Uicker, Portland, OR (US); Laura Schaeffer, East Lansing, MI (US)

Correspondence Address: MEDLEN & CARROLL, LLP;Suite 350, 101 Howard Street, San Francisco, CA, 94105, US

(73) Assignee: MICHIGAN STATE UNIVERSITY

(21) Appl. No.: 11/434,081

(22) Filed: May 15, 2006

Related U.S. Application Data
(60) Provisional application No. 60/684,857, filed on May 26, 2005.

(51) Int. Cl. C12Q 1/68; C07H 21/04; C12N 9/22

(52) U.S. Cl. 435/6; 435/199; 536/24.1

(57) ABSTRACT

The present invention discloses that essential and highly conserved GTPases (i.e., for example, YlqF, YqeH, YsxC, or YphC proteins) from Bacillus subtilis participate in ribosome biogenesis. Before this invention, the biological function of the GTPase proteins were unknown. These GTPase proteins are disclosed to affect translation and believed to promote ribosome biogenesis. For example, cells depleted of YlqF protein accumulate a precursor of the 50S subunit that migrates at 45S, suggesting a role in assembly of the large ribosomal subunit. Analysis of the protein content of the 45S particle showed that ribosomal protein L16 is missing from the large subunit. Inhibitors of GTPase-ribosomal interactions (i.e., for example, ribosomal protein or ribosomal nucleic acids) comprise a novel class of antimicrobial compounds.

### FIELD OF INVENTION

The present invention is related to the development of new antimicrobials. In one embodiment, the invention relates to a GTPase that controls the assembly of microbial ribosomal subunits (i.e., for example, 30S, 45S, or 50S subunits). In one embodiment, a GTPase interacts with ribosomal proteins (i.e., for example, L16, L35, or L36) or ribosomal nucleic acids (i.e., for example, 5S, 16S, or 23S rRNA). In one embodiment, the present invention contemplates the identification of antimicrobial compounds that may prevent the interaction of the GTPase proteins with ribosomal proteins or ribosomal nucleic acids and may prevent the function of GTPases in ribosome biogenesis.

### BACKGROUND

The ribosome is responsible for a fundamental process common to all cells; the synthesis of proteins. The mechanisms by which ribosomes are formed in vivo still remain to be elucidated. In contrast to the over 150 non-ribosomal proteins identified in eukaryotes as important for ribosome biogenesis, very few proteins have been implicated in this process in bacteria.

Ribosomes are complex structures comprised of over 50 proteins and 3 RNA molecules. Ban et al., “The complete atomic structure of the large ribosomal subunit at 2.4 A resolution” Science 289:905-920 (2000); Bashan et al., “Structural basis of the ribosomal machinery for peptide bond formation, translocation, and nascent chain progression” Mol Cell 11:91-102 (2003); and Ramakrishnan V., “Ribosome structure and the mechanism of translation” Cell 108:557-572 (2002). The details of how ribosomes are formed in vivo, however, remains an open question.

Small (30S) and large (50S) bacterial ribosomal subunits can be assembled in vitro under non-physiological conditions (high temperatures, high salt). These conditions, however, suggest that additional factors are necessary for in vivo assembly. Culver G. M., “Assembly of the 30S ribosomal subunit” Biopolymers 68:234-249 (2003).; and Nierhaus K. H., “The assembly of prokaryotic ribosomes” Biochimie 73:739-755 (1991). The factors playing a dedicated role in bacterial ribosome assembly (i.e., prokaryotic) have not been uncovered, and unlike eukaryotic systems, the current state of the art suggests that non-ribosomal proteins or enzymes are not involved. Dez et al., “Ribosome synthesis meets the cell cycle” Curr Opin Microbiol 7:631-637 (2004); and Culver G. M., “Assembly of the 30S ribosomal subunit” Biopolymers 68:234-249 (2003).

GTPases are non-ribosomal proteins known to control a wide variety of processes including protein synthesis. For example, GTPases are known to control protein translation during initiation (initiation factor 2), elongation (EF-Tu, EF-G), and termination (RF1 and RF2). In addition, several GTPases from yeast (i.e., for example, Nog1p, Nog2p and Nug1p) have been implicated in eukaryotic 60S biogenesis or transport. Bassler et al., “Identification of a 60S preribosomal particle that is closely linked to nuclear export” Mol Cell 8:517-529 (2001); Jensen et al., “The NOG1 GTP-binding protein is required for biogenesis of the 60S ribosomal subunit” J Biol Chem 278:32204-32211 (2003); Kallstrom et al., “The putative GTPases Nog1p and Lsg1p are required for 60S ribosomal subunit biogenesis and are localized to the nucleus and cytoplasm, respectively” Mol Cell Biol 23:4344-4355 (2003); and Saveanu et al., “Nog2p, a putative GTPase associated with pre-60S subunits and required for late 60S maturation steps” Embo J 20:6475-6484 (2001). Bacterial genome sequencing projects have shown that most bacteria typically contain several G proteins (i.e., for example, a GTPase). The function of these enzymes are, however, unknown (i.e., for example, at least eleven in B. subtilis). Kunst et al., “The complete genome sequence of the gram-positive bacterium Bacillus subtilis” Nature 390, 249-256 (1997)).

The identification and regulation of bacterial proteins responsible for the assembly of bacterial ribosomes is a problem currently facing the field of microbiology. What is needed is the identification of non-ribosomal bacterial proteins that play a role in bacterial ribosomal protein biogenesis. These proteins will be targets for non-ribosomal bacterial protein inhibitors that may be developed into a new class of antimicrobial pharmaceutical agents.

### SUMMARY

The present invention is related to the development of new antimicrobials. In one embodiment, the invention relates to a GTPase that controls the assembly of microbial ribosomal subunits (i.e., for example, 30S, 45S, or 50S subunits). In one embodiment, a GTPase interacts with ribosomal proteins (i.e., for example, L16, L35, or L36) or ribosomal nucleic acids (i.e., for example, 5S, 16S, or 23S rRNA). In one embodiment, the present invention contemplates the identification of antimicrobial compounds that may prevent the interaction of the GTPase proteins with ribosomal proteins or ribosomal nucleic acids and may prevent the function of GTPases in ribosome biogenesis.

In one embodiment, the present invention contemplates a method to identify an antimicrobial compound, comprising: a) providing; i) a test compound; ii) a first protein, said first protein comprising a GTPase selected from the group comprising YlqF, YsxC, or YphC; iii) a second protein capable of interacting with said first protein, wherein said second protein comprises a ribosomal protein. In one embodiment, the ribosomal protein is selected from the group consisting of L16, L35, and L36; b) mixing said first and second protein in the presence of said test compound; and c) measuring the interaction of said first and second proteins. In one embodiment, the YlqF protein is a eukaryotic homolog. In one embodiment, said homolog comprises a human Sprn protein.

In one embodiment, the present invention contemplates a method to identify an antimicrobial compound, comprising: a) providing; i) a test compound; ii) a protein, said protein comprising a GTPase selected from the group comprising YlqF, YsxC, YphC, and YqeH; and iii) a nucleic acid capable of interacting with said protein, wherein said nucleic acid comprises a ribosomal nucleic acid; b) mixing said first protein and said nucleic acid in the presence of said test compound, and c) measuring the interaction of said protein and said nucleic acid. In one embodiment, said nucleic acid comprises ribonucleic acid (RNA). In one embodiment, said ribonucleic acid may be selected from the group comprising 5S, 16S, or 23S RNA.

In one embodiment, the present invention contemplates a method to identify an antimicrobial compound, comprising: a) providing: i) a test compound; ii) a first protein, said first protein comprising a GTPase; iii) a second protein capable of interacting with said first protein, wherein said second protein comprises a ribosomal protein; b) mixing said first and second protein in the presence of said test compound; and c) measuring the interaction of said first and second proteins. In one embodiment, the GTPase is selected from the group consisting of YlqF, Era, Obg, YsxC, YphC, YyaF, YlaG, YlaG, YloQ, ThdF, and YnbA. In one embodiment, the ribosomal protein is selected from the group consisting of L16, L35, and L36. In one embodiment, the second protein comprises at least one fluorophore. In one embodiment, the measuring comprises energy emission detection. In one embodiment, the energy emission comprises FRET. In another embodiment, the measuring comprises GTPase activity detection. In one embodiment, the test compound comprises a protein translation inhibitor. In one embodiment, the protein translation inhibitor binds to said GTPase. In one embodiment, the binding prevents said interaction between said GTPase and said ribosomal protein. In one embodiment, the protein translation inhibitor is selected from the group consisting of a polypeptide and a small molecular weight organic molecule.

In one embodiment, the present invention contemplates a method to identify an antimicrobial compound, comprising: a) providing: i) a test compound; ii) a protein comprising a GTPase; iii) a nucleic acid capable of interacting with said protein, wherein said nucleic acid comprises a ribosomal nucleic acid; b) mixing said protein and nucleic acid in the presence of said test compound; and c) measuring the interaction of said protein and nucleic acid. In one embodiment, the GTPase is selected from the group consisting of YlqF, YqeH, Era, Obg, YsxC, YphC, YyaF, YlaG, YlaG, YloQ, ThdF, and YnbA In one embodiment, the ribosomal nucleic acid is selected from the group consisting of 5S, 16S, or 23S rRNA. In one embodiment, the nucleic acid comprises at least one fluorophore. In one embodiment, the measuring comprises energy emission detection. In one embodiment, the energy emission comprises FRET. In one embodiment, the measuring comprises GTPase acitivity detection. In one embodiment, the test compound comprises a protein translation inhibitor. In one embodiment, the protein translation inhibitor binds to said GTPase. In one embodiment, the binding prevents said interaction between said GTPase and said nucleic acid. In one embodiment, the protein translation inhibitor is selected from the group consisting of a polypeptide and a small molecular weight organic molecule.

In one embodiment, the present invention contemplates a composition comprising a GTPase, a ribosomal protein, a ribosomal RNA, and a test compound, wherein said GTPase comprises a basic N-terminus and an acidic C-terminus. In one embodiment, the ribosomal protein interacts with said C-terminus. In one embodiment, the ribosomal RNA interacts with said N-terminus. In one embodiment, the test compound binds with said GTPase. In one embodiment, the GTPase is selected from the group consisting of YlqF, YqeH, Era, Obg, YsxC, YphC, YyaF, YlaG, YloQ, ThdF, and YnbA. In one embodiment, the ribosomal protein is selected from the group consisting of L16, L35, and L36. In one embodiment, the ribosomal RNA is selected from the group comprising 5S, 16S, or 23S rRNA. In one embodiment, the test compound comprises a protein translation inhibitor. In one embodiment, the binding prevents said interaction between said GTPase and said ribosomal protein. In one embodiment, the protein translation inhibitor is selected from the group consisting of a polypeptide and a small molecular weight organic molecule.

In one embodiment, the present invention contemplates a method comprising: a) providing; i) a microarray comprising a B. subtilis genome; ii) a ribosomal protein having an attached first fluorophore and second fluorophore; iii) a third fluorophore capable of binding to said ribosomal protein; and iv) a GTPase protein capable of interacting with said ribosomal protein; b) contacting said microarray with said ribosomal protein and said GTPase protein in the presence of said third fluorophore; and c) measuring an energy emission. In one embodiment, the energy emission comprises FRET. In one embodiment, the method further comprises a test compound capable of binding to said GTPase protein. In one embodiment, said test compound reduces said energy emission. In one embodiment, said ribosomal protein is selected from the group comprising L16, L35, and L36. In one embodiment, said GTPase is selected from the group comprising YlqF, YqeH, Era, Obg, YsxC, YphC, YyaF, YlaG, YloQ, ThdF, and YnbA.

In one embodiment, the present invention contemplates a method comprising: a) providing; i) a microarray comprising a B. subtilis genome; ii) a reporter gene incorporated into the genome, wherein said gene is expressed under a GTPase protein-depleted condition; iii) a small molecule capable of inhibiting said GTPase protein; b) incubating said genome with said reporter gene and said small molecule; and c) measuring said reporter gene expression. In one embodiment, the GTPase protein is selected from the group comprising YlqF, YqeH, YsxC, and YphC. In one embodiment, the reporter gene comprises green fluorescent protein. In one embodiment, the reporter gene comprises a luciferase protein.

### DEFINITIONS

The terms used in this invention are, in general, expected to adhere to standard definitions generally accepted by those having ordinary skill in the art of microbiology. A few exceptions, as listed below, have been further defined within the scope of the present invention.

The term “antimicrobial compound” as used herein, refers to any compound that reduces microbial growth. Such a compound includes, but is not limited to, polypeptides, proteins, small molecular weight organic molecules, hormones, and the like.

The term “test compound” as used herein, refers to any compound suspected of having a capability of interacting with a GTPase that results in antimicrobial activity. For example, a “test compound” includes, but is not limited to, protein translation inhibitors, metabolic inhibitors, polypeptides, small molecular weight organic molecules, hormones, and the like.

The term “GTPase” as used herein, refers to any protein or polypeptide that hydrolyzes guanosine triphosphate (GTP) into guanosine diphosphate+phosphate+free energy. For example, a GTPase may be used to release energy in order to promote ribosome biogenesis.

The term “GTP-binding protein” refers to any protein capable of binding guanosine triphoshpate and/or guanosine diphosphate.

The term “ribosomal protein” as used herein, refers to any polypeptide that is assembled into a functional ribosome. For example, a bacterial ribosomal protein includes, but is not limited to, L16, L35, or L36.

The term “ribosomal nucleic acid” as used herein, refers to any nucleic acid that is assembled into a functional ribosome. For example, a bacterial nucleic acid includes, but is not limited to, 5S, 16S, or 23S rRNA.

The term “proteomic analysis” as used herein, refers to a biochemical analysis that isolates and identifies specific proteins present in a biological sample. For example, a sucrose centrifugation density gradient technique may be used which separates proteins based upon sedimentation rates. Generally, the term “proteomics” refers to a branch of biotechnology concerned with applying the techniques of molecular biology, biochemistry, and genetics to analyzing the structure, function, and interactions of the proteins produced by the genes of a particular cell, tissue, or organism, with organizing the information in databases, and with applications of the data (as in medicine or biology).

The term “polypeptide” as used herein, refers to a condensed polymer of amino acids within which each amino acid is joined by a peptide bond to the immediately preceding and to the immediately subsequent amino acid in the chain. A polypeptide comprises a first amino acid, generally referred to as the amino-terminal amino acid, and a last amino acid, generally referred to as the carboxyl terminal amino acid. Natural polypeptides include, but are not limited to, linear, branched, or cyclic forms. Generally, a polypeptide comprises a mixture of twenty naturally occurring amino acids. Nonetheless, an amino acid may be covalently modified. There is considerable overlap between a “polypeptide” and a “protein”.

The term “protein translation” as used herein, refers to the process whereby free amino acids are enzymatically condensed into peptidergic polymers, thus forming polypeptides and proteins. The formation of peptide bonds is facilitated by intracellular structures (ribosomes) that provide support and enzymatic control for the polymer synthesis.

The term “small molecular weight organic molecule” refers to any protease-resistant compound capable of interacting with a polypeptide or protein. For example, a small molecular weight organic molecule may range between approximately 5-1,500 daltons, preferably between 100-750 daltons, and more preferably between 250-500 daltons.

The term “interacting” or “binding” refers to any physical relationship between at least two molecules, wherein said physical relationship may be stabilized by forces including, but not limited to, ionic bonding, covalent bonding, hydrophobic forces, Van der Waals forces, electrostatic attraction, and the like.

The term “microarray” as used herein, refers to any solid surface comprising a plurality of addressed biological macromolecules (e.g., nucleic acids or antibodies). The location of each of the macromolecules in the microarrayis known, so as to allow for identification of the samples following analysis.

### BRIEF DESCRIPTION OF THE FIGURES

The Figures identified below are only presented as illustrations of the present invention and are not intended to be limiting.

FIG. 1 presents illustrative data regarding B. subtilis growth having the following mutations. Both Plates—Region A: Pₛₚₐₙₖ -ylqF (Strain RB301); Region B: Pₛₚₐₙₖ -yqeH (Strain RB286); and Region C: Pₛₚₐₙₖ -aroD (Strain RB288). All bacterial cultures were grown on LB medium. The left plate was supplemented with 1 mM isopropyl-β-D-thiogalactopyranoside (IPTG).

FIG. 2 presents one embodiment of the genome structure of an yqeH gene region. Arrows represent genes and direction of transcription. The location of the Pₛₚₐₙₖ promoters in Strain RB286 and Strain RB288 are indicated by the 90° arrows.

FIG. 3 presents exemplary data showing ribosome profiling of B. subtilis cells depleted of YlqF, IF2, and EF-Tu proteins using the Pₛₚₐₙₖ inducible promoter. The traces are presented from the bottom of a sucrose gradient (25%) on the left to the top (10%) on the right. Panel A: Pₛₚₐₙₖ -ylqF+1 mM IPTG. Panel B: Pₛₚₐₙₖ -ylqF. Panel C: Pₛₚₐₙₖ -IF2. Panel D. Pₛₚₐₙₖ -EF-Tu. Panel E. wild type genome. Elution Order left-to-right: 70S-50S-30S.

FIG. 4 presents exemplary data showing that the ratio of 23S:16S rRNA is altered in Pₛₚₐₙₖ -yqeH cells (Strain RB286) in various concentrations of IPTG. Total RNA was run on a 1% formaldehyde agarose gel. Error bars depict the standard error of the mean (SEM) for three independent experiments. Solid Bar: 0 μM IPTG. Crosshatched Bar: 5 μM IPTG. Vertical Striped Bar: 10 μM IPTG. Horizontal Striped Bar: 1000 μM IPTG.

FIG. 5 presents exemplary data showing ribosome profiling of YqeH protein-depleted B. subtilis cells using the Pₛₚₐₙₖ inducible promoter. Panel A: Pₛₚₐₙₖ -yqeH+1 mM IPTG. Panel B: Pₛₚₐₙₖ -yqeH. Panel C. Close-up of Pₛₚₐₙₖ -yqeH 30S region; not to scale.

FIG. 6 presents an exemplary gel electrophoretic purification of YlqF-His₆ and YqeH-His₆ proteins. Lane 1: molecular weight standards. Lane 2: ylqF-His₆ (˜32 kD). Lane 3. YqeH-His₆ (˜41 kD).

FIG. 7 shows a representative growth pattern of Strain RB406 (wild type; Plate A-C Left Side) versus Strain RB406 (0.03% xylose suppressor mutant; Plate A-C Right Side) using different concentrations of xylose. Plate A: LB media+2.0% xylose. Plate B: LB media+0.03% xylose. Plate C: LB media only. Suppression is seen only in the presence of 0.03% xylose (i.e., Plate B).

FIG. 8 presents illustrative photomicrographs showing nucleoid morphology of cells depleted of YlqF proteins. Simultaneous fluorescent/phase contrast microscopy allowed visualization of both cell and nucleoid morphology. Nucleoids were stained with 4,6-diamidino-2-phenylindole (DAPI). Panel A. Pₛₚₐₙₖ -ylqF+1 mM IPTG. Panel B. Pₛₚₐₙₖ -ylqF. Panel C: B. subtilis wild type cells+tetracycline. Note that YlqF protein-depleted cells (Panel B) appear longer than wild type cells (Panel C).

FIG. 9 presents exemplary data regarding ribosome profiles of cells depleted of YlqF, IF2, or EF-Tu proteins. Panel A: Pₛₚₐₙₖ-ylqF+1 mM IPTG. Panel B: Pₛₚₐₙₖ -ylqF (i.e., cells depleted of YlqF protein). Panel C: Pₛₚₐₙₖ -infB (i.e., cells depleted of initiation factor 2 protein). Panel D. Pₛₚₐₙₖ -tufA (i.e., cells depleted of EF-Tu protein). Gradient Profile: Bottom (25%); Top: (10%). Panel E. Wild-type. 70S, 50S and 30S subunits elute in left-to-right order, respectively.

FIG. 10 presents exemplary data regarding the ribosome profiles of Strain RB301 (i.e., Pₛₚₐₙₖ -ylqF) grown in varying concentrations of IPTG. Doubling times are indicated within parenthesis; Panel A. No IPTG (150 minutes). Panel B. 6 μM (90 minutes). Panel C. 10 μM (50 minutes). Panel D. 20 μM (35 minutes). Panel E. 1 mM (25 minutes). Gradients were analyzed by continuous monitoring of A254. Gradient Profile: Bottom (25%); Top: (10%). Dashed lines, left-to-right, represent gradient positions for migration of 70S, 50S and 30S complexes, respectively.

FIG. 11 presents a representative 12% SDS-PAGE electrophoresis gel showing that the ribosomal protein L16 is missing in the 45S subunit. Proteins of the 45S subunit Lane 1: 45S subunit. Lane 2: 50S subunit (lane 2). Arrows indicate proteins bands that are present in the 50S particle and are missing from the 45S particle. The identity of L16 determined by mass spectrometry. The proteins in the low molecular weight region of the gel have not been identified.

FIG. 12 presents one embodiment of a proposed model for YlqF protein function in ribosomal biogenesis.

FIG. 13 presents exemplary data regarding ribosome profiles of cells depleted of YsxC, YphC, YlqF, or initiation factor 2 proteins. All strains grown without IPTG. Panel A: Pₛₚₐₙₖ-ysxC (i.e., cells depleted of YsxC protein). Panel B: Pₛₚₐₙₖ -yphC (i.e., cells depleted of YphC protein). Panel C: Pₛₚₐₙₖ -ylqF (i.e., cells depleted of YlqF protein). Panel D. Pₛₚₐₙₖ -infB (i.e., cells depleted of initiation factor 2 protein). Gradient Profile: Bottom (25%); Top: (10%). 70S, 50S and 30S subunits elute in left-to-right order, respectively.

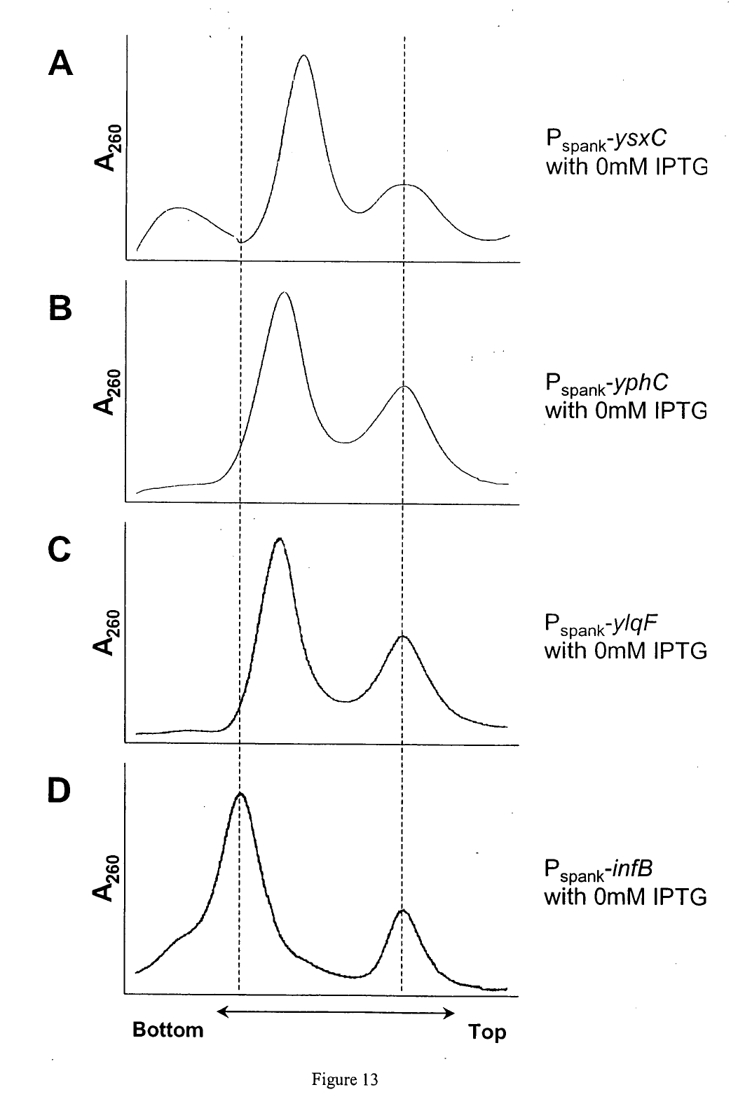

FIG. 14 presents exemplary data of an electrophoretic gel separation showing ribosomal protein compositions: Lane A: 45S subunits from YsxC protein-depleted cells; Lane B: 50S subunits from a Pₛₚₐₙₖ-yphC strain+1 mM IPTG; Lane C: 45S subunits from YphC protein-depleted cells; Top Arrow: ribosomal protein L16; Bottom Arrow: ribosomal proteins L35 and L36. Note: Ribosomal proteins L16, L35, and L36 are missing in Lane A and Lane C.

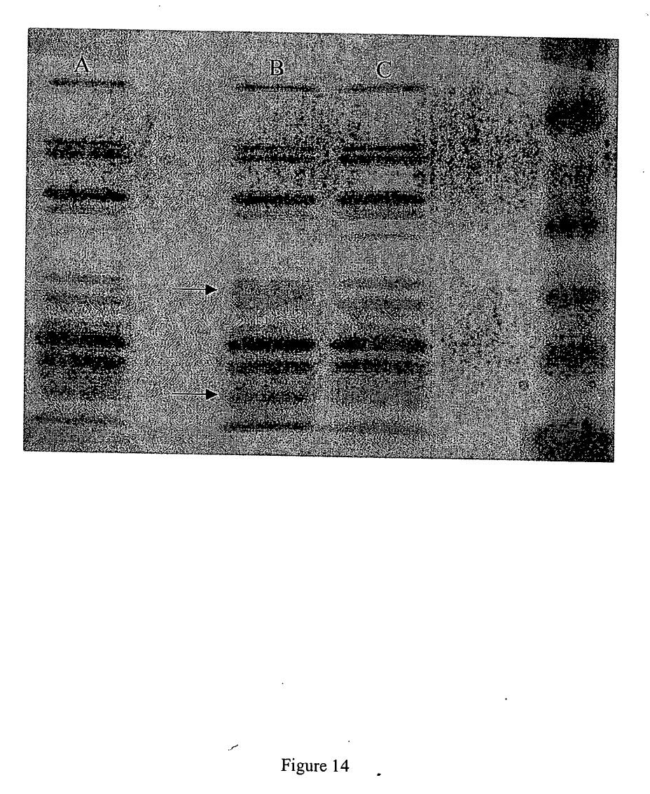

FIG. 15 presents exemplary data demonstrating that the 70S ribosomes of partially YlqF-depleted cells are composed of 50S and 30S subunits. Panel A: Stippled box identifies the 70S subunits isolated from partially depleted RB301 (Pₛₚₐₙₖ-ylqF) cells. Panel B: Wild-type subunits from non-depleted cells. Panel C: Dissociated 70S ribosomes from Panel A (stippled box and arrow) using a 10-25% sucrose gradient. The dashed lines indicate where 70S, 50S, and 30S complexes migrate in the gradient.

FIG. 16 presents exemplary data showing that YlqF directly interacts with the 45S intermediate and not the mature 50S subunit. The data was collected from 12% SDS-PAGE electrophoresis and blotted to a nylon membrane using standard Western Blot analysis. Incubations using rabbit polyclonal YlqF antibody was followed by horseradish peroxidase-conjugated goat anti-rabbit antibody and Western Lightning® (PerkinElmer) chemiluminescent detection. Purified YlqF-His6 was added as a comparative marker.

### DETAILED DESCRIPTION OF THE INVENTION

The present invention is related to the development of new antimicrobials. In one embodiment, the invention relates to a GTPase that controls the assembly of microbial ribosomal subunits (i.e., for example, 30S, 45S, or 50S subunits). In one embodiment, a GTPase interacts with ribosomal proteins (i.e., for example, L16, L35, or L36) or ribosomal nucleic acids (i.e., for example, 5S, 16S, or 23S rRNA). In one embodiment, the present invention contemplates the identification of antimicrobial compounds that may prevent the interaction of the GTPase proteins with ribosomal proteins or ribosomal nucleic acids and may prevent the function of GTPases in ribosome biogenesis.

In one embodiment, the present invention contemplates the identification of antimicrobial compounds that may prevent the interaction of the YlqF protein and ribosomal proteins or may prevent the function of YlqF in ribosome biogenesis GTP-binding proteins (i.e., for example, a GTPase) may function as molecular switches and might be involved in many essential cellular processes in prokaryotes and eukaryotes. Bourne, H. R., “GTPases: a family of molecular switches and clocks” Philos Trans R Soc Lond B Biol Sci 349:283-9 (1995); Bourne et al., “The GTPase superfamily: a conserved switch for diverse cell functions” Nature 348:125-32 (1990); Bourne et al., “The GTPase superfamily: conserved structure and molecular mechanism” Nature 349:117-27 (1991); and Vetter et al., “The guanine nucleotide-binding switch in three dimensions” Science 294:1299-304 (2001). One known GTPase is Ras, which is mutated in several types of human cancers. Macaluso et al., “Ras family genes: an interesting link between cell cycle and cancer” J Cell Physiol 192:125-30 (2002). Research related to the role of Ras proteins and the functions of other members of the Ras superfamily have shown they may play critical roles in the cell including development, vesicle trafficking, and apoptosis. Boettner et al., “The role of Rho GTPases in disease development” Gene 286:155-74 (2002); Manser E., “Small GTPases take the stage” Dev Cell 3:323-8 (2002): and Martinez et al., “Ras proteins” Biochim Biophys Acta 1404:101-12 (1998). In bacteria, GTPase are believed to play roles in many processes including, but not limited to, translation, protein secretion, and cell division. Elongation factor proteins (i.e., for example, EF-Tu or EF-G) and initiation factor 2 (IF2) have been identified as GTPase proteins involved in translation. Ramakrishnan V., “Ribosome structure and the mechanism of translation” Cell 108:557-72 (2002); and Rodnina et al., “GTPases mechanisms and functions of translation factors on the ribosome” Biol Chem 381:377-87 (2000).

I. GTPases

In one embodiment, present invention contemplates that GTPases may be involved in ribosome biogenesis or translation initiation. Some GTPases having an unusual GTP-binding domain structure are believed conserved in gram-positive bacteria (i.e., for example, YlqF and YqeH proteins) with homologs found in many eukaryotic genomes, including humans. In one embodiment, the present invention contemplates elucidating the roles of ylqF, ysxC, yphC and yqeH genes in bacteria.

Although it is not necessary to understand the mechanism of an invention, it is believed that many G proteins (i.e., for example, a GTPase) are essential for bacterial growth. Morimoto et al., “Six GTP-binding proteins of the Era/Obg family are essential for cell growth in Bacillus subtilis” Microbiology 148:3539-3552 (2002). Recent studies of the essential GTPase proteins, Era and Obg (CgtA), have demonstrated that they interact with the ribosome and may function in translation or ribosome assembly. Sharma et al., “Interaction of Era with the 30S Ribosomal Subunit Implications for 30S Subunit Assembly” Mol Cell 18:319-29 (2005).

Further, the primary function of Era protein is unclear because the protein is also implicated in carbon metabolism, cell cycle progression, and growth rate control. Similar to Era, CgtA depletion or mutation in C. crescentus causes a decrease in 50S ribosomal subunit formation. Datta et al., “The Caulobacter crescentus GTPase CgtAC is required for progression through the cell cycle and for maintaining 50S ribosomal subunit levels” Mol Microbiol 54:1379-1392 (2004); Inoue et al., “Suppression of defective ribosome assembly in a rbfA deletion mutant by overexpression of Era, an essential GTPase in Escherichia coli” Mol Microbiol 48:1005-1016 (2003); Lin et al., “The Caulobacter crescentus CgtAC protein cosediments with the free 50S ribosomal subunit” J Bacteriol 186:481-489 (2004); Sayed et al., “Era, an essential Escherichia coli small G-protein, binds to the 30S ribosomal subunit” Biochem Biophys Res Commun 264:51-54 (1999); Scott et al., “The Bacillus subtilis GTP binding protein obg and regulators of the sigma(B) stress response transcription factor cofractionate with ribosomes” J Bacteriol 182:2771-2777 (2000); Tan et al., “Overexpression of two different GTPases rescues a null mutation in a heat-induced rRNA methyltransferase” J Bacteriol 184:2692-2698 (2002); and Wout et al., “The Escherichia coli GTPase CgtAE cofractionates with the 50S ribosomal subunit and interacts with SpoT, a ppGpp synthetase/hydrolase” J Bacteriol 186:5249-5257 (2004). Cell viability of a CgtA temperature sensitive mutant, however, does not correlate with the observed 50S subunit levels suggesting that the essential function of CgtA is not 50S biogenesis. Also, Obg (CgtA) has also been shown to participate in stress responses, chromosome partitioning, DNA replication, and a newly identified replication fork arrest checkpoint. Datta et al., “The Caulobacter crescentus GTPase CgtAC is required for progression through the cell cycle and for maintaining 50S ribosomal subunit levels” Mol Microbiol 54:1379-1392 (2004); Foti et al., “A bacterial G protein-mediated response to replication arrest” Mol Cell 17: 549-560 (2005); Kobayashi et al., “Deficiency of essential GTPbinding protein ObgE in Escherichia coli inhibits chromosome partition” Mol Microbiol 41:1037-1051 (2001); and Wout et al., “The Escherichia coli GTPase CgtAE cofractionates with the 50S ribosomal subunit and interacts with SpoT, a ppGpp synthetase/hydrolase” J Bacteriol 186:5249-5257 (2004). Thus, the precise functions of Era and Obg proteins in ribosome assembly, if any, remain a mystery.

GTPase proteins have also been implicated in diverse cellular processes including, but not limited to, DNA replication, replication fork arrest response, chromosome partitioning, carbon metabolism, and development. Britton et al., “Characterization of mutations affecting the Escherichia coli essential GTPase era that suppress two temperature-sensitive dnaG alleles” J Bacteriol 179:4575-4582 (1997); Britton et al., “Cell cycle arrest in Era GTPase mutants: a potential growth rate-regulated checkpoint in Escherichia coli” Mol Microbiol 27:739-750 (1998); Dutkiewicz et al., “Overexpression of the cgtA (yhbZ, obgE) gene, coding for an essential GTP-binding protein, impairs the regulation of chromosomal functions in Escherichia coli” Curr Microbiol 45:440-445 (2002); Foti et al., “A bacterial G protein-mediated response to replication arrest” Mol Cell 17: 549-560 (2005); Gollop et al., “A GTP-binding protein (Era) has an essential role in growth rate and cell cycle control in Escherichia coli” J Bacteriol 173:2265-2270 (1991); Inoue et al., “Specific growth inhibition by acetate of an Escherichia coli strain expressing Era-dE, a dominant negative Era mutant” J Mol Microbiol Biotechnol 4:379-388 (2002); Lemer et al., “Pleiotropic changes resulting from depletion of Era, an essential GTP-binding protein in Escherichia coli” Mol Microbiol 5:951-957 (1991); Minkovsky et al., “Bex, the Bacillus subtilis homolog of the essential Escherichia coli GTPase Era, is required for normal cell division and spore formation” J Bacteriol 184:6389-6394 (2002); Powell et al., “Novel proteins of the phosphotransferase system encoded within the rpoN operon of Escherichia coli. Enzyme IIANtr affects growth on organic nitrogen and the conditional lethality of an era^{ts} mutant” J Biol Chem 270:4822-4839 (1995); and Vidwans et al., “Possible role for the essential GTP-binding protein Obg in regulating the initiation of sporulation in Bacillus subtilis” J Bacteriol 177:3308-3311 (1995). At this time, however, the precise roles of many bacterial GTP-binding proteins in the cell remain unclear.

FtsY and Ffh are GTPase proteins that are believed part of the signal recognition particle that is involved in protein secretion. Herskovits et al., “New prospects in studying the bacterial signal recognition particle pathway” Mol Microbiol 38:927-39 (2000); and Powers et al., “Reciprocal stimulation of GTP hydrolysis by two directly interacting GTPases” Science 269:1422-4 (1995). Further, the GTPase, FtsZ, may be involved in division septum wherein the polymerization of this protein is regulated by its GTP/GDP bound state. Lutkenhaus et al., “Bacterial cell division and the Z ring” Annu Rev Biochem 66:93-116 (1997).

A. Essential Bacterial GTPases

As described above, several bacterial genes that have been identified by those having skill in the art as essential for bacterial growth. The term “essential”, as used herein, means that those having skill in the art have identified that the expression of a particular gene is necessary for bacterial growth and/or survival. Many experiments that are offered to support this conclusion are limited to in vitro cell culture and in vivo knock-out genetic engineering techniques. These technologies, however, have not identified any particular structural compositions that can be used to identify compounds to inhibit the activity of these genes. In one embodiment, the present invention contemplates that compounds that prevent the interaction of a GTPase and a specific ribosomal protein and/or rRNA may be used to inhibit the biological activity of these GTPase proteins.

Currently, at least thirty-six (36) genes have been reported as essential for the growth of Streptococcus pneumoniae and some other bacterial species (i.e., Staphylococcus aeurus, Haemophilus influenzae, and Escherichia coli). Some genes encode GTPase proteins that may include, but are not limited to, obg, ylqF, yphC, yqeH, and ysxC genes. Zalacain et al., “A Global Approach To Identify Novel Broad-Spectrum Antibacterial Targets Among Proteins Of Unknown Function” J Mol Microbiol Biotechnol 6:109-126 (2003); and Fritz et al. “Use Of YLQF, YAEG, YYBQ, And YSXC, Essential Bacterial Genes And Polypeptides” U.S. Pat. No. 6,815,177. Filed: Apr. 25, 2003. Issued: Nov. 9, 2004 (herein incorporated by reference). While this research suggests that these genes are essential for bacterial growth and might be potential targets for antibiotics, they do not disclose that essential gene products (i.e., for example, those products of the ylqF, ysxC, yphC or yqeH genes) are capable of interacting with any ribosomal proteins or ribosomal nucleic acids.

The GTPase proteins era and obg are believed essential for cell viability and may couple growth with cell cycle progression. Chein et al., “Staphylococcal GTPase OBG Nucleotide Sequence Encoding Staphylococcal GTP-Binding Protein” U.S. Pat. No. 6,706,495. Filed: Feb. 23, 2001. Mar. 16, 2004; and Nicholas R. O., “ERA” U.S. Pat. No. 6,225,102. Filed: Mar. 14, 2000. Issued: May 1, 2001 (both herein incorporated by reference). Chein et al., however, admits that even though the obg gene encodes a known GTPase protein, its biological interactions are unknown. Nicholas believes the intracellular interactions of era are responsible for bacterial septation and nucleoid segregation. Specifically, neither reference teaches that any GTPase proteins (i.e., for example, YlqF or YqeH) are involved in ribosomal biogenesis. The FtsZ protein is also a GTPase that may be useful in identifying antimicrobial compounds that interfere with cell division. There are no teachings, however, that suggest the FtsZ protein is involved in ribosomal biogenesis. de Boer et al., “Compositions And Methods For Screening Antimicrobials” U.S. Pat. No. 5,948,889. Filed: May 21, 1996. Issued: Sep. 7, 1999 (herein incorporated by reference). The functionality of B. subtilis homologs for many of the GTPase proteins, are however, unknown.

Analysis of the Bacillus subtilis genome has identified at least eleven highly conserved putative GTP-binding proteins of unknown function, many of which are conserved in eukaryotes. These eleven GTPases are suggested to be members of the “translation factor related” (TRAFAC) class. Leipe et al., “Classification and evolution of P-loop GTPases and related ATPases” J Mol Biol 317:41-72 (2002). Experimental, evolutionary, and bioinformatics analyses of the bacterial TRAFAC GTPases have led to the speculation that many of these proteins are involved in translation. Caldon et al., “Function of the universally conserved bacterial GTPases” Curr Opin Microbiol 6:135-9 (2003); Caldon et al., “Evolution of a molecular switch: universal bacterial GTPases regulate ribosome function” Mol Microbiol 41:289-97 (2001); and Leipe et al., “Classification and evolution of P-loop GTPases and related ATPases” J Mol Biol 317:41-72 (2002). These GTPase proteins may have possible functions ranging from direct roles in translation to acting as sensors of ribosome activity or status. Studies of several of these GTPases in different microorganisms have demonstrated that several of these proteins are essential for bacterial growth. Maddock et al., “Identification of an essential Caulobacter crescentus gene encoding a member of the Obg family of GTP-binding proteins” J Bacteriol 179:6426-31 (1997); Morimoto et al., “Six GTP-binding proteins of the Era/Obg family are essential for cell growth in Bacillus subtilis” Microbiology 148:3539-52 (2002); Takiff et al., “Genetic analysis of the rnc operon of Escherichia coli” J Bacteriol 171:2581-90 (1989); Trach et al., “The Bacillus subtilis spo0B stage 0 sporulation operon encodes an essential GTP-binding protein” J Bacteriol 171:1362-71 (1989). The YlqF and YqeH proteins form subfamilies within the yawG-ylqF gene family, each comprising GTPase proteins of unknown function. The human homolog of the YawG protein (not found in bacteria) has been localized in the nucleolus which has led to speculation that members of this family are involved in translation. Racevskis et al., “Cloning of a novel nucleolar guanosine 5′-triphosphate binding protein autoantigen from a breast tumor” Cell Growth Differ 7:271-80 (1996). Table 1.

TABLE 1
Conserved GTPase Proteins of Unknown Function In B. subtilis

| | | Conserved in | Eukaryotic |
|---|---|---|---|
| GTPase | Essential? | prokaryotes? | homolog? |
| Era | Yes | Yes | Yes |
| Obg (CgtA) | Yes | Yes | Yes |
| YsxC | Yes | Yes | Yes |
| ylqF | Yes | Yes* | Yes |
| YphC (EngA) | Yes | Yes | No |
| YyaF | No | Yes | Yes |
| YlaG | No | Yes | No |
| yqeH | Yes | Yes* | Yes |
| YloQ (YjeQ) | Yes | Yes | No |
| ThdF | ND | Yes | Yes |
| YnbA | ND | Yes | Yes |

( ): Alternative name in other organisms.
*Found in many gram positive bacteria and some gram-negative bacteria.

Genetic era mutations, or cellular Era protein depletion, have implicated era in cell division, cell cycle control, carbon metabolism, and rRNA processing. Britton et al., “Characterization of mutations affecting the Escherichia coli essential GTPase era that suppress two temperature-sensitive dnaG alleles” J Bacteriol 179:4575-82 (1997); Britton et al., “Cell cycle arrest in Era GTPase mutants: a potential growth rate-regulated checkpoint in Escherichia coli” Mol Microbiol 27:739-50 (1998); Inoue et al., “Suppression of defective ribosome assembly in a rbfA deletion mutant by overexpression of Era, an essential GTPase in Escherichia coli” Mol Microbiol 48:1005-16 (2003); Inoue et al., “Specific growth inhibition by acetate of an Escherichia coli strain expressing Era-dE, a dominant negative era mutant” J Mol Microbiol Biotechnol 4:379-88 (2002); Johnstone et al., “The widely conserved Era G-protein contains an RNAbinding domain required for Era function in vivo” Mol Microbiol 33:1118-31 (1999); Lerner et al., “Pleiotropic changes resulting from depletion of Era, an essential GTP-binding protein in Escherichia coli” Mol Microbiol 5:951-7 (1991); Lu et al., “The gene for 16S rRNA methyltransferase (ksgA) functions as a multicopy suppressor for a cold-sensitive mutant of era, an essential RAS-like GTP-binding protein in Escherichia coli” J Bacteriol 180:5243-6 (1998); Meier et al., “16S rRNA is bound to era of Streptococcus pneumoniae” J Bacteriol 181:5242-9 (1999); and Minkovsky et al., “Bex, the Bacillus subtilis homolog of the essential Escherichia coli GTPase Era, is required for normal cell division and spore formation” J Bacteriol 184:6389-94 (2002).

The essential GTPase proteins, Era and Obg (CgtA), have also been implicated in ribosome biogenesis or stability, and cells depleted of these proteins exhibit a decrease in 70S ribosome formation. Inoue et al., “Suppression of defective ribosome assembly in a rbfA deletion mutant by overexpression of Era, an essential GTPase in Escherichia coli” Mol Microbiol 48:1005-1016 (2003); and Lin et al., “The Caulobacter crescentus CgtAC protein cosediments with the free 50S ribosomal subunit” J Bacteriol 186:481-489 (2004). Era-depleted cells accumulate 17S rRNA indicating that the 16S small subunit rRNA is not processed correctly, however, ribosome assembly intermediates have not been detected. Inoue et al., “Suppression of defective ribosome assembly in a rbfA deletion mutant by overexpression of Era, an essential GTPase in Escherichia coli” Mol Microbiol 48:1005-1016 (2003).

Similarly, obg genetic defects also cause pleiotropic phenotypes and have implicated the obg gene in ribosome assembly, stress activation, chromosome partitioning, DNA repair, and DNA replication. Kok et al., “Effects on Bacillus subtilis of a conditional lethal mutation in the essential GTP-binding protein Obg” J Bacteriol 176:7155-60 (1994); Lin et al., “The Caulobacter crescentus CgtA(C) Protein Cosediments with the Free 50S Ribosomal Subunit” J Bacteriol 186:481-9 (2004); Scott et al., “Obg, an essential GTP binding protein of Bacillus subtilis, is necessary for stress activation of transcription factor sigma(B)” J Bacteriol 181:4653-60 (1999); Sikora-Borgula et al., “A role for the common GTP-binding protein in coupling of chromosome replication to cell growth and cell division” Biochem Biophys Res Commun 292:333-8 (2002); Slominska et al., “Impaired chromosome partitioning and synchronization of DNA replication initiation in an insertional mutant in the Vibrio harveyi cgtA gene coding for a common GTP-binding protein” Biochem J 362:579-84 (2002); Vidwans et al., “Possible role for the essential GTP-binding protein Obg in regulating the initiation of sporulation in Bacillus subtilis” J Bacteriol 177:3308-11 (1995); Wout et al., “The Escherichia coli GTPase CgtAE cofractionates with the 50S ribosomal subunit and interacts with SpoT, a ppGpp synthetase/hydrolase” J Bacteriol 186:5249-57 (2004); and Zielke et al., “Involvement of the cgtA gene function in stimulation of DNA repair in Escherichia coli and Vibrio harveyi” Microbiology 149:1763-70 (2003).

Recent studies have suggested that the primary functions of Era and Obg proteins may involve ribosome assembly or ribosomal RNA processing/modification. Inoue et al., “Suppression of defective ribosome assembly in a rbfA deletion mutant by overexpression of Era, an essential GTPase in Escherichia coli” Mol Microbiol 48:1005-16 (2003); Lin et al., “The Caulobacter crescentus CgtA(C) Protein Cosediments with the Free 50S Ribosomal Subunit” J Bacteriol 186:481-9 (2004); and Tan et al., “Overexpression of two different GTPases rescues a null mutation in a heat-induced rRNA methyltransferase” J Bacteriol 184:2692-8 (2002). Likewise, analysis of the essential GTPase gene yjeQ in Escherichia coli (i.e., yloQ in B. subtilis) suggests this gene may be associated with ribosomes. Daigle et al., “Studies of the interaction of Escherichia coli yjeQ with the ribosome in vitro” J Bacteriol 186:1381-7 (2004).

B. Bacterial GTPases and Ribosomal Biogenesis

The present state of the art makes clear that some bacterial GTPase proteins have unknown function. In one embodiment, the present invention contemplates an essential GTPase in Bacillus subtilis (i.e., for example, ylqF and yqeH) that interacts with a ribosomal protein (i.e., for example, L16, L35, or L36) or a ribosomal nucleic acid (i.e., for example, 5S, 16S, or 23S rRNA). In one embodiment, the interaction of a GTPase protein and a ribosomal protein and/or a ribosomal ribonucleic acid occurs during ribosome biogenesis. Although it is not necessary to understand the mechanism of an invention, it is believed that DNA microarray expression analysis and the cell biology of YlqF protein-depleted cells indicate that YlqF proteins supports protein translation. It is further believed that defective ribosomal formation occurs in YlqF protein-depleted cells, wherein the primary function of YlqF protein appears to support 50S large subunit biogenesis because a 45S assembly intermediate accumulates in ylqF deficient cells. Supporting this potential mechanism are other observations that in Saccharomyces cerevisiae, eukaryotic YlqF, YsxC, or YphC protein homologs might be involved in the biogenesis or transport of a 60S large ribosomal subunit.

In another embodiment, bacterial cells depleted in YqeH proteins fail to accumulate 70S ribosomes. Although it is not necessary to understand the mechanism of an invention, it is believed that the primary defect in YqeH protein-depleted cells appears to be reflected by the lack of accumulation of the 30S subunit. It is further believed that isolated rRNA analysis from YqeH protein-depleted cells demonstrate that 16S rRNA levels are decreased nearly 50% when compared to wild-type cells.

In one embodiment, the present invention contemplates a method comprising; providing a GTPase protein, wherein said GTPase controls ribosome biogenesis. In one embodiment, the GTPase controls ribosome biogenesis by sensing intracellular GTP levels. In one embodiment, the ribosome biogenesis comprises either the 50S subunit or the 70S subunit formation. In one embodiment, 50S subunit formation is reduced in YlqF protein-depleted cells. In another embodiment, 70S ribosome formation is reduced in YlqF protein-depleted or YqeH protein-depleted cells. In yet another embodiment, bacterial cell growth rate is reduced in YlqF protein-depleted or YqeH protein-depleted cells. Although it is not necessary to understand the mechanism of an invention, it is believed that that YlqF protein or YqeH protein may be limiting growth factors when either is underexpressed.

Recently, it was reported that rRNA synthesis may be controlled by GTP concentration in Bacillus subtilis. For example, a decreased GTP concentration leads to reduced transcription of rRNA and less ribosome biogenesis. Krasny et al., “An alternative strategy for bacterial ribosome synthesis: Bacillus subtilis rRNA transcription regulation” Embo J 23, 4473-4483 (2004). Although it is not necessary to understand the mechanism of an invention, it is believed that when a reduction in intracellular energy is sensed (i.e., for example, reduced GTP levels) a rapid reduction in protein synthesis may occur by a combination of reduced initiation of ribosome biogenesis and reduced assembly of partially completed ribosomes.

In one embodiment, the present invention contemplates regulating ribosome biogenesis at a stage wherein 70S ribosome formation immediately halts. In one embodiment, ribosome formation is halted under fast growth conditions (i.e., for example, wherein ˜50% of cell mass comprises ribosomes). Although it is not necessary to understand the mechanism of an invention, it is believed that immediately halting ribosome biogenesis allows the cell to quickly limit the amount of functional ribosomes when it encounters adverse conditions. For example, reduced intracellular GTP concentration (i.e., for example, as observed during a “stringent response” or decreasing resource availability) would signal a YlqF protein to cease assembly of the 50S subunit. It is further believed that since a YlqF protein may be essential for bacterial growth, the protein serves as a “checkpoint” (i.e., for example, a sensor) to determine if intracellular GTP levels are sufficient to warrant assembling additional ribosomes. In one embodiment, the present invention contemplates that a YlqF protein triggers progression from a 45S assembly intermediate to an active 50S subunit. In one embodiment, a YlqF protein receives an intracellular environmental signal to initiate additional protein translation activity. In one embodiment, an intracellular environmental signal comprises intracellular GTP levels. In another embodiment, an intracellular environmental signal comprises an intracellular metabolic intermediate.

The present invention contemplates that ylqF and yqeH genes may be essential for bacterial growth. For example, neither gene can be deleted from the bacterial genome without providing the cell with a second (i.e., alternative) gene copy. It is known that both proteins can bind GTP and GDP. Morimoto et al., “Six GTP-binding proteins of the Era/Obg family are essential for cell growth in Bacillus subtilis” Microbiology 148:3539-52 (2002). In one embodiment, aylqF gene affects bacterial cell morphology. In one embodiment, a YlqF protein-depleted bacterial cell become slightly longer in size and comprise condensed nucleoids. It is known that nucleoid condensation also occurs when translation is inhibited. Donachie et al., “Chromosome partition in Escherichia coli requires postreplication protein synthesis” J Bacteriol 171:5405-9 (1989); van Helvoort et al., “Chloramphenicol causes fusion of separated nucleoids in Escherichia coli K-12 cells and filaments” J Bacteriol 178:4289-93 (1996).

In one embodiment, the present invention contemplates that a yqeH gene negatively regulates DNA replication. It is known that YqeH protein-depleted cells contain additional chromosomes as compared to wild-type cells. Morimoto et al., “Six GTP-binding proteins of the Era/Obg family are essential for cell growth in Bacillus subtilis” Microbiology 148:3539-52 (2002). Despite these observations, the bacterial intracellular functions of both ylqF and yqeH genes are still unknown.

1. The ylqF Gene

In one embodiment, the present invention contemplates that a ylqF gene promotes ribosome biogenesis. In one embodiment, a ylqF gene increases protein translation. In one embodiment, a ylqF gene product interacts with a ribosomal protein or ribosomal nucleic acid (i.e., for example, 5S or 23S rRNA) in a late step of ribosome biogenesis. In another embodiment, a ylqF gene product improves protein translation initiation. In another embodiment, a ylqF gene product is an essential GTPase protein found in B. subtilis.

It is known that YlqF proteins are members of the TRAFAC superfamily of GTPases and P-loop ATPases and is conserved in all three kingdoms of life. Leipe et al., “Classification and evolution of P-loop GTPases and related ATPases” J Mol Biol 317:41-72 (2002). ylqF is believed to be widely distributed in Gram-positive bacteria and is also found sporadically in some Gram-negative bacteria, including, but not limited to, Vibrio cholerae and Neisseria meningiditis, but not Escherichia coli. Although ylqF genes have been shown to be essential for growth in Bacillus subtilis, Streptococcus pneumoniae, and Staphylococcus aureus, its biological function has not been elucidated. Morimoto et al., “Six GTP-binding proteins of the Era/Obg family are essential for cell growth in Bacillus subtilis” Microbiology 148:3539-3552 (2002); and Zalacain et al., “A global approach to identify novel broad-spectrum antibacterial targets among proteins of unknown function” J Mol Microbiol Biotechnol 6:109-126 (2003). A recent survey of proteins identified a “top ten” list that require functional characterization. Galperin et al., “Conserved hypothetical’ proteins: prioritization of targets for experimental study” Nucleic Acids Res 32:5452-5463 (2004). Three proteins identified as requiring functional characterization were highly conserved GTPases; including, for example, YlqF and YsxC proteins.

In one embodiment, the present invention contemplates that YlqF protein promotes protein translation, wherein ribosome biogenesis is increased. Although it is not necessary to understand the mechanism of an invention, it is believed that YlqF protein-depleted cells accumulate a 50S subunit precursor that migrates at approximately 45S. It is further believed that the 45S precursor accumulation suggests a role for YlqF protein in large ribosomal subunit assembly. In one embodiment, the 45S precursor is missing ribosomal protein L16.

2. Eukaryotic YlqF Protein Homologs

The present invention contemplates that essential GTPases in bacteria may be involved in ribosome biogenesis. In eukaryotic studies, GTPases involved in ribosome biogenesis have been also linked to cell cycle events. Dez et al., “Ribosome synthesis meets the cell cycle” Curr Opin Microbiol 7:631-637 (2004).

A similar link may also be present between bacterial ribosome biogenesis and cell cycle progression. The present invention contemplates that since several of the essential bacterial GTPases have eukaryotic counterparts the elucidation of their functions may provide insight into understanding ribosome biogenesis and sensing of translational status in higher organisms.

Eukaryotic homologs of YlqF and YqeH proteins are known in the art. There is increasing evidence in eukaryotic systems that the signals from the nucleolus, where ribosome biogenesis takes place, can couple cell growth with the cell cycle and differentiation. Racevskis et al., “Cloning of a novel nucleolar guanosine 5′-triphosphate binding protein autoantigen from a breast tumor” Cell Growth Differ 7:271-80 (1996); Tsai et al., “A nucleolar mechanism controlling cell proliferation in stem cells and cancer cells” Genes Dev 16:2991-3003 (2002). The nucleolar GTPase nucleostemin, which is a distant homolog of the YlqF protein, controls proliferation of stem cells and cancer cells. Because of the homology between YlqF protein and eukaryotic nucleolar GTPase proteins, uncovering the function of YlqF protein in B. subtilis may aid our understanding of eukaryotic GTPase proteins.

It is known that many eukaryotic organisms have YlqF protein homologs suggesting their involvement in large subunit (i.e., 60S) assembly. For example, the archaeal ribosomal protein L10e (L10 in eukaryotes) is homologous to the bacterial L16 and they adopt similar structures. Nishimura et al., “Solution structure of ribosomal protein L16 from Thermus thermophilus HB8” J Mol Biol 344:1369-1383 (2004). Similarly, the location of L10e in the structure of the archaeon Haloarcula marismortui 50S subunit is similar to where L16 is located in the D. radiodurans 50S subunit. Ban et al., “The complete atomic structure of the large ribosomal subunit at 2.4 A resolution” Science 289:905-920 (2000); Harms et al., “High resolution structure of the large ribosomal subunit from a mesophilic eubacterium” Cell 107:679-688 (2001). In one embodiment, the present invention contemplates that eukaryotic ribosome assembly may be regulated by YlqF protein-homologs that control L10e (L10) insertion into the ribosome. In another embodiment, YlqF protein homologs promote mitochondrial ribosome biogenesis. It is known that the Mtg1 gene product is required for mitochondrial translation. Barrientos et al., “MTG1 codes for a conserved protein required for mitochondrial translation. Mol Biol Cell 14:2292-2302 (2003).

Eukaryotic homologs of GTPases may be identified by BLAST and PSI-BLAST analysis. Analysis of eukaryotic Era protein in humans, chickens, and plants suggest roles for Era protein in development and apoptosis. Akiyama et al., “Mammalian homologue of E. coli Ras-like GTPase (ERA) is a possible apoptosis regulator with RNA binding activity” Genes Cells 6:987-1001 (2001); Britton et al., “Isolation and preliminary characterization of the human and mouse homologues of the bacterial cell cycle gene era” Genomics 67:78-82 (2000); Gohda et al., “Elimination of the vertebrate Escherichia coli Ras-like protein homologue leads to cell cycle arrest at G1 phase and apoptosis” Oncogene 22:1340-8 (2003); and Ingram et al., “The Antirrhinum erg gene encodes a protein related to bacterial small GTPases and is required for embryonic viability” Curr Biol 8:1079-82 (1998). Despite these observations, the precise functions of these GTPase genes are not known in any biological system.

YlqF protein is believed to be evolutionarily related to eukaryotic 60S ribosome subunit biogenesis factors. BLAST and PSI-BLAST analyses of the non-redundant protein database demonstrated an evolutionary link between YlqF and eukaryotic ribosomal proteins involved in large subunit biogenesis or proteins that are localized to the nucleolus. Table 2.

TABLE 2
BLAST and PSI-BLAST analysis of eukaryotic proteins
with significant similarity to YlqF protein.

| | | | BLAST^{A} | PSI-BLAST^{B} |
|---|---|---|---|---|
| Protein | Species | Function | E-value | E-value |
| Nog2p | S. | 60S ribosome | 1e−13 | 2e−10 |
| | cerevisiae | biogenesis | | |
| Nug1p | S. | 60S ribosome | 5e−12 | 1e−08 |
| | cerevisiae | biogenesis | | |
| Sprn | Humans | Unknown | 8e−36 | 1e−20 |
| GNL2 | Humans | Unknown | 1e−11 | 1e−14 |
| GNLN3 | Humans | unknown | 1e−12 | 2e−05 |
| Nucleostemin | Humans | Nucleolar | 5e−09 | 2e−02 |
| | | location - | | |
| | | regulates | | |
| | | cell | | |
| | | differentiation | | |
| | | and | | |
| | | proliferation | | |

^{A}Blast analysis determined using full length ylqF protein of B. subtilis versus the non-redundant protein database. Only selected results are shown from humans and yeast. Many additional eukaryotic species have homologs of ylqF.
^{B}PSI-Blast analysis was carried out using the C-terminal 105 amino acids of ylqF consisting of the domain of the protein structurally distinct from the GTPbinding domain. Three iterations were performed.

YlqF protein is known to have similarity to the Nog2p GTP-binding domain; a eukaryotic Sacchromycetes cerevisiae GTPase that may be involved in 60S ribosome biogenesis. Saveanu et al., “Nog2p, a putative GTPase associated with pre-60S subunits and required for late 60S maturation steps” Embo J 20:6475-6484 (2001). The YlqF protein is also homologous to Nug1p from S. cerevisiae, another eukaryotic GTPase, that when mutated, causes accumulation of pre-60S ribosomal subunits in the cell. Bassler et al., “Identification of a 60S preribosomal particle that is closely linked to nuclear export” Mol Cell 8:517-529 (2001). The similarities between YlqF protein and some eukaryotic GTPase proteins are not confined to conserved GTP-binding domains. For example, PSI-BLAST analysis of the non-redundant database using only the C-terminal 105 amino acids (excluding the GTP-binding domain) showed significant similarity to the eukaryotic GTPases listed in Table 1. Nug1p and Nog2p amino acid sequences (i.e., eukaryotic large subunit biogenesis factors) found homology to YlqF proteins in many bacteria. It is believed that Homo sapiens YlqF protein homologs include, but are not limited to, Sprn, nucleostemin, and the nucleolar GTPase GNL2. For example, nucleostemin is a nucleolar GTPase proposed to coordinate cell proliferation and growth. Tsai et al., “A nucleolar mechanism controlling cell proliferation in stem cells and cancer cells” Genes Dev 16, 2991-3003 (2002). Although it is not necessary to understand the mechanism of an invention, it is believed that Sprn, nucleostemin, GNL2, or GNL3 may regulate human ribosomal biogenesis.

3. Pₛₚₐₙₖ-ylqF Bacterial Strains

In one embodiment, the present invention contemplates bacterial strains comprising an inducible ylqF gene. In another embodiment, the ylqF gene comprises an inducible LacI repressible promoter Pₛₚₐₙₖ. In one embodiment, the Pₛₚₐₙₖ promoter is induced by isopropyl-beta-D-thiogalactopyranoside (IPTG). In one embodiment, a B. subtilis strain RB301 comprises the inducible ylqF gene. Although it is not necessary to understand the mechanism of an invention, it is believed that the Pₛₚₐₙₖ strain construction comprises the removal of a native full-length ylqF gene promoter and inserts the inducible Pₛₚₐₙₖ promoter. It is further believed that the ylqF gene is likely monocistronic since it is flanked on both sides by putative transcription terminators, consequently potential polar effects on downstream gene expression is not likely a factor. In the absence of IPTG, the Pₛₚₐₙₖ promoter is inactive, wherein YlqF protein is not produced. DNA microarray results confirmed that expression of genes directly downstream of the ylqF gene were not affected (data not shown).

4. The yqeH Gene

In one embodiment, the present invention contemplates that the yqeH gene regulates bacterial 30S ribosomal subunit biogenesis. Although it is not necessary to understand the mechanism of an invention, it is believed that the level of 16S rRNA is decreased in YqeH protein-depleted cells, resulting in a decrease in the amount of 30S subunit present in the cell, wherein 70S ribosome levels are decreased with a concomitant reduction in bacterial cell growth rate. In one embodiment, YqeH protein regulates the processing or modification of the small subunit RNA. In another embodiment, YqeH protein regulates 30S subunit assembly wherein 16S rRNA is decreased.

II. Ribosomal Proteins

Using techniques known in the art, it is possible to analyze proteins missing from the large subunit in YlqF protein-depleted cells. Although it is not necessary to understand the mechanism of an invention, it is believed that this analysis may provide an explanation of how YlqF protein may regulate translation by participating in a late ribosome assembly step. In one embodiment, ribosomal protein L16 is missing from the 50S subunit in YlqF protein-depleted cells. It is known that L16 is one of the last proteins added during in vitro assembly of the 50S subunit. Franceschi et al., “Ribosomal proteins L15 and L16 are mere late assembly proteins of the large ribosomal subunit. Analysis of an Escherichia coli mutant lacking L15” J Biol Chem 265:16676-16682 (1990).

Biochemical and structural studies of the ribosome have demonstrated that L16 is involved in multiple ribosome functions. The position of L16 in the ribosome shows close proximity to helices of the 23S rRNA that play important roles in peptidyl-transferase activity and IF2 binding. Ban et al., “The complete atomic structure of the large ribosomal subunit at 2.4 A resolution” Science 289:905-920 (2000); Harms et al., “High resolution structure of the large ribosomal subunit from a mesophilic eubacterium” Cell 107:679-688 (2001); La Teana et al., “Initiation factor IF 2 binds to the alpha-sarcin loop and helix 89 of Escherichia coli 23S ribosomal RNA” RNA 7:1173-1179 (2001). It is believed that L16 makes contact with an aminoacylated tRNA at the ribosomal A site. Bashan et al., “Structural basis of the ribosomal machinery for peptide bond formation, translocation, and nascent chain progression” Mol Cell 11:91-102 (2003); and Nishimura et al., “Solution structure of ribosomal protein L16 from Thermus thermophilus HB8” J Mol Biol 344:1369-1383 (2004). These structural data are supported by biochemical evidence showing a role for L16 in peptidyltransferase activity and association of the 50S and 30S subunits. Bernabeu et al., “The involvement of protein L16 on ribosomal peptidyl transferase activity” Eur J Biochem 79:469-472 (1977); Guerin et al., “Effects of partial deproteinization on the functional properties of 50S ribosomal subunits of E. coli. Biochimie 63:699-707 (1981); and Tate et al., “The peptidyltransferase centre of the Escherichia coli ribosome. The histidine of protein L16 affects the reconstitution and control of the active centre but is not essential for release-factor-mediated peptidyl-tRNA hydrolysis and peptide bond formation” Eur J Biochem 165:403-408 (1987). Although it is not necessary to understand the mechanism of an invention, it is believed that the addition of L16 results in a large conformational change in the 50S subunit that may contribute to the altered migration of the 45S complex observed in the ribosome profiling experiments. Teraoka et al., “Protein L16 induces a conformational change when incorporated into a L16-deficient core derived from Escherichia coli ribosomes” FEBS Lett 88, 223-226 (1978).

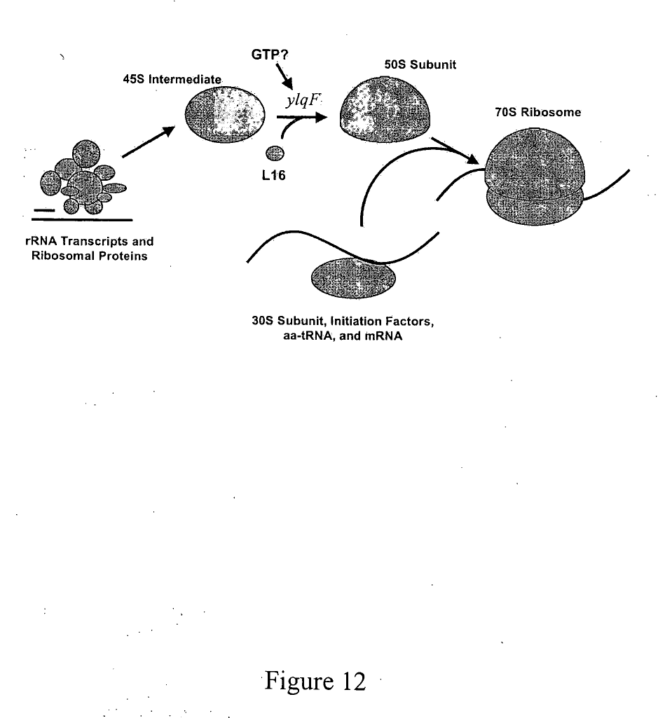

In one embodiment, the present invention contemplates that YlqF protein controls translation by regulating the incorporation of L16 into the 50S subunit. FIG. 12. Although it is not necessary to understand the mechanism of an invention, it is believed that L16 causes a conformational change in the region of the 50S subunit that then allows for the formation of an IF2 binding site, interaction with the A-site tRNA, and correct formation of the peptidyltransferase center. Further, it is believed that YlqF protein control of L16 incorporation allows the cell to easily modulate translational capacity by regulating a protein that affects ribosomal structure and function.

III. Ribosomal Assembly

Ribosomes are the site of action of many antimicrobial agents. Essential proteins involved in ribosomal assembly and function are contemplated as within the scope of the present invention and may be useful in developing a new class of antimicrobial agents (i.e., for example, antibiotics). In one embodiment, a GTPase protein (i.e., for example, YlqF and YqeH proteins) promotes ribosome biogenesis and consequently, induces protein translation. In another embodiment, an inhibitor of bacterial GTPase enzyme prevents ribosome biogenesis and consequently, inhibits protein translation. Although it is not necessary to understand the mechanism of an invention, it is believed that YlqF and YqeH proteins are the first non-ribosomal bacterial proteins shown to be essential for in vivo ribosome subunit biogenesis.

According to the “RNA world” theory of ribosome evolution, RNA sequences were first able to promote peptide bond formation and were later incorporated into more efficient RNA-protein complexes. Jeffares et al., “Relics From The RNA World” Mol. Evol. 46:18-36 (1998). Although rRNA sequences have not been precisely conserved throughout evolution, the basic structural features of the ribosome are highly conserved in all kingdoms. Gray et al., “Evolution Of rRNA Gene Organization” pp. 49-69. In: Ribosomal RNA: Structure, Evolution, Processing And Function In Protein Biosynthesis; Eds: R. A. Zimmermann and A. E. Dahlberg, CRC Press, Boca Raton, Fla. (1996). The structure and function of bacterial ribosomes can be subjected to crystal structure analysis. Brodersen et al., “Crystal Structure Of The 30S Ribosomal Subunit From Thermus thermophilus: Structure Of The Proteins And Their Interactions With 16S RNA” J. Mol. Biol. 316:725-768 (2002); Ramakrishnan, V. “Ribosome Structure And The Mechanism Of Translation” Cell 108:557-572 (2002); and Yusupov et al., “Crystal Structure Of The Ribosome At 5.5 A Resolution” Science 292:883-896 (2001). Despite this knowledge, the processing and assembly of transcripts into mature rRNA species and overall ribosome biogenesis is still very poorly understood.

This lack of understanding is particularly prevalent in prokaryotes. Three rRNA genes often lie within ribosomal DNA (rDNA) operons in a specific sequence: for example, small-subunit rRNA, large-subunit rRNA, and 5S rRNA. However, this organization can vary. For example, in Thermus thermophilus, the 16S gene is separated from, and transcribed independently of, the 23S and 5S genes. Grindley et al. “Effects Of Different Alleles Of The E. coli K12 polA Gene On The Replication Of Non-Transferring Plasmids” Mol. Gen. Genet. 143:311-318 (1976). In Pirellula marina, however, the SS genes are separated from the 16S and 23S rRNA genes, while all three rRNAs are transcribed separately in both Leptospira interrogans and Thermoplasma acidophilum. Fukunaga et al., “Unique Organization Of Leptospira interrogans rRNA Genes” J. Bacteriol. 171:5763-5767 (1989); Liezack et al, “Evidence For Unlinked rrn Operons In The Planctomycete Pirellula marina” J. Bacteriol. 171:5025-5030 (1989): and Ree et al., “Organization And Expression Of The 16S, 23S And 5S Ribosomal RNA Genes From The Archaebacterium Thermoplasma acidophilum” Nucleic Acids Res. 18:4471-4478 (1990).

In other organisms, ribosomal gene organization can also be highly fragmented. For example, pieces of the rRNA genes of mitochondrial genomes in green algae are encoded in separate regions of the mitochondrial genome but details describing how ribosomes are made from multiple pieces of rRNA are lacking. Nedelcu et al., “The Complete Mitochondrial DNA Sequence Of Scenedesmus obliquus Reflects An Intermediate Stage In The Evolution Of The Green Algal Mitochondrial Genome” Genome Res. 10:819-831 (2000); and Schnare et al., “Fourteen Internal Transcribed Spacers In The Circular Ribosomal DNA Of Euglena gracilis” J. Mol. Biol. 215:85-91 (1990).

A. Small Subunit Processing

Maturation of 23S and 16S rRNA is derived mainly from studies in E. coli; little is known how this process occurs in other organisms. Small ribosome subunits including, but not limited to, 23S, 16S, and 5S rRNA are believed transcribed as a single transcript (i.e., for example, the 30S subunit) that may be subsequently cleaved into individual mature forms by ribonucleases (RNAses). For example, RNAseIII performs the initial cleavage of the 30S transcript into immature 23S and 17S precursors. Srivastava et al., “Mechanism and regulation of bacterial ribosomal RNA processing” Annu Rev Microbiol 44:105-29 (1990).

The immature 23S rRNA that is released by RNAseIII cleavage comprises 3-7 nucleotides on the 5′ end and 7-9 nucleotides on the 3′ end. It is believed that functional ribosomes can be reconstituted using the immature 23S molecule, indicating that additional processing of these nucleotides is not essential for ribosome-mediated protein translation.

In contrast, the 17S rRNA undergoes several additional processing events resulting in the removal of an additional 115 nucleotides from the 5′ end and 33 nucleotides from the 3′ end. The production of the mature 16S rRNA results from cleavage by an as yet unidentified RNAase. Li et al., “RNase G (CafA protein) and RNase E are both required for the 5′ maturation of 16S ribosomal RNA” Embo J 18:2878-85 (1999). In further contrast to the 23S subunit, mature 16S rRNA is essential for ribosome function; in other words, immature 16S rRNA cannot form functional ribosomes.

B. Ribosomal Protein Assembly

The bacterial ribosome (i.e., for example, E. coli) is a large and dynamic ribonucleoprotein machine composed primarily of two subunits. The 50S large subunit is believed formed by two RNAs (23S and 5S) and thirty-three (33) ribosomal proteins (L1 to L36), whereas the small 30S subunit comprises one RNA (16S) and twenty-one (21) ribosomal proteins (S1 to S21).

Ribosomal RNA (rRNA) is thought to be synthesized as a large precursor, the maturation of which may involve numerous steps, including nucleotide modification and endo- and exonucleolytic cleavages that sequentially remove precursor sequences. Srivastava et al., “Mechanism And Regulation Of Bacterial Ribosomal RNA Processing” Annu Rev Microbiol 44:105-129 (1990). The ribosomal proteins assemble onto the rRNAs concomitantly during synthesis and subsequent processing yields functional subunits. Although the structure of the mature ribosome has now been characterized at the atomic level, the molecular details of assembly and processing remain incompletely understood. Ramakrishnan V., “Ribosome Structure And The Mechanism Of Translation” Cell 108:557-572 (2002).

Initial processing of rRNA in Escherichia coli is carried out by the endonuclease RNase III, which separates the 16S, 23S, and 5S rRNAs and any tRNAs that are encoded in the operon. Apirion et al., “Molecular Biology Of RNA Processing In Prokaryotic Cells” pp. 36-52. In: Processing of RNA, Ed: Apirion, D., CRC Press, Inc., Boca Raton, Fla. (1984); French et al., “Transcription Mapping Of The Escherichia coli Chromosome By Electron Microscopy” J. Bacteriol. 171:4207-4216 (1989); and Hofmann et al., “Visualization Of Ribosomal Ribonucleic Acid Synthesis In A Ribonuclease III-Deficient Strain Of Escherichia coli” J. Bacteriol. 132:718-722 (1977). Presumably, the structural integrity of the mature RNA molecules in ribosomal subunits is maintained by secondary and tertiary structure, as well as by cooperation with ribosomal proteins, resulting in functional ribosomes. Intervening sequence (IVS) elements are mostly found in two sites of eubacterial 23S rRNA, at nucleotides 533 to 560 (helix 25) and 1164 to 1185 (helix 45). Burgin et al., “The Excision Of Intervening Sequences From Salmonella 23S Ribosomal RNA” Cell 60:405-414 (1990); and Mattatall et al., “Salmonella enterica serovar Typhimurium LT2 Possesses Three Distinct 23S rRNA Intervening Sequences” J. Bacteriol. 178:2272-2278 (1996). These preferred positions might reflect selection against the presence of IVS elements at rRNA functional sites, ribosomal protein binding sites, or sites where fragmentation of the RNA molecule would cause instability of the 50S subunit. Interestingly, in an RNase III mutant strain, uncleaved IVS elements are phenotypically silent, as their presence does not affect growth or incorporation of the longer 23S rRNA into fully functional ribosomes. Gregory et al., “Functional Escherichia coli 23S rRNAs Containing Processed And Unprocessed Intervening Sequences From Salmonella typhimurium” Nucleic Acids Res. 24:4918-4923 (1996).

The prokaryotic ribosomal protein L16 is known as essential for ribosomal assembly. Sumpter et al., “Modification Of Histidine Residues On Protein From The 50S Subunit Of The Escherichia coli Ribosome. Effects On The Subunit Assembly And Peptidyl Transferase Centre Activity” Eur J Biochem 196:255-260 (1991). It is known that modification of histidine residues result in an unstable ribosomal constitution despite the fact that 60-70% of peptidyl transferase activity was maintained. Until the present invention, GTPase proteins (i.e., for example, YsxC and YphC) were not suggested as having any involvement in E. coli ribosomal biogenesis by interacting with L16.

In vivo, the assembly of ribosomal subunits is a stepwise process involving a series of intermediate precursor particles. These intermediates contain a subset of ribosomal proteins as well as precursors of rRNA, and sediment more slowly than the mature ribosomal subunits. Lindahl L., “Intermediates And Time Kinetics Of The In Vivo Assembly Of Escherichia coli Ribosomes” J Mol Biol 92: 15-37 (1975). Functional E. coli subunits can be reconstituted from isolated rRNA and ribosomal proteins in vitro, indicating that reconstitution is a self-assembly process. Traub et al., “Structure And Function Of E. coli Ribosomes. V. Reconstitution Of Functionally Active 30S Ribosomal Particles From RNA And Proteins” Proc Natl Acad Sci USA 59:777-784 (1968); and Dohme et al., “Total Reconstitution And Assembly Of 50S Subunits From Escherichia coli Ribosomes In Vitro” J Mol Biol 107:585-599 (1976). The reconstitution studies revealed a well-defined order of addition of the ribosomal proteins and a substantial co-operativity in their binding. The precursor particles formed in vitro have a protein composition similar to those found in vivo, suggesting that assembly proceeds similarly both in vivo and in vitro. Nierhaus K., “The Assembly Of Prokaryotic Ribosomes” Biochimie 73:739-755 (1991).

Ribosomes are a known major component of the cell and their synthesis is tightly controlled. Ribosomes are complex structures comprising three RNA molecules and at least fifty (50) proteins that are assembled in an ordered manner. Under optimal growth conditions, ribosomes can account for nearly 50% of cell mass. Neidhardt et al., Physiology of the Bacterial Cell: A Molecular Approach, Sinauer Associates (1990). Thus, a cell devotes a significant amount of its energy to producing translational capacity. For instance, ribosomal RNA must be processed and modified, wherein many modifications (i.e., for example, pseudourididylation and methylation) are important for ribosome function. Lane et al., “Pseudouridine and O2′-methylated nucleosides. Significance of their selective occurrence in rRNA domains that function in ribosome-catalyzed synthesis of the peptide bonds in proteins” Biochimie 77:7-15 (1995); Ofengand J., “Ribosomal RNA pseudouridines and pseudouridine synthases” FEBS Lett 514:17-25 (2002); and Srivastava et al., “Mechanism and regulation of bacterial ribosomal RNA processing” Annu Rev Microbiol 44:105-29 (1990). Recently, it was estimated that approximately 25% of genes essential for growth in yeast may be involved in ribosome biogenesis or translation. Peng et al., “A panoramic view of yeast noncoding RNA processing” Cell 113:919-33 (2003). In contrast to eukaryotes, the control of bacterial (i.e., prokaryotic) ribosome biogenesis by non-ribosomal proteins is not well understood. Until the present invention, no non-ribosomal protein essential for bacterial ribosome biogenesis had been identified. Although 30S subunits from E. coli can be assembled in vitro with purified proteins and mature 16S rRNA, “additional factors” are likely to participate in vivo. Culver G. M., “Assembly of the 30S ribosomal subunit” Biopolymers 68:234-49 (2003).

Candidates for these “additional factors” may include the DnaK protein chaperone system, among others. Maki et al., “The DnaK chaperone system facilitates 30S ribosomal subunit assembly’ Mol Cell 10:129-38 (2002); and Maki et al., “Demonstration of the role of the DnaK chaperone system in assembly of 30S ribosomal subunits using a purified in vitro system” RNA 9:1418-21 (2003): RbfA, Bylund et al., “RimM and RbfA are essential for efficient processing of 16S rRNA in Escherichia coli” J Bacteriol 180:73-82 (1998); and Xia et al., “The role of RbfA in 16S rRNA processing and cell growth at low temperature in Escherichia coli” J Mol Biol 332:575-84 (2003): SrmB, Charollais et al., “The DEAD-box RNA helicase SrmB is involved in the assembly of 50S ribosomal subunits in Escherichia coli” Mol Microbiol 48:1253-65 (2003): and CsdA, Charollais et al., “CsdA, a cold-shock RNA helicase from Escherichia coli, is involved in the biogenesis of 50S ribosomal subunit” Nucleic Acids Res 32:2751-9 (2004). None, however, are essential for in vivo ribosomal biogenesis because cells with mutations in these genes are still viable.

However, whereas in vivo assembly takes only a few minutes at 37° C., the reconstitution of active subunits in vitro requires much longer incubation times, high temperatures, and non-physiological ionic conditions. These discrepancies suggest that, as yet unidentified, non-ribosomal proteins assisting in vivo ribosomal assembly are lacking in in vitro ribosomal assembly techniques. Only recently has the existence of one such an ‘assembly factor’ been demonstrated. It has been shown that purified DnaK and its co-chaperones DnaJ and GrpE proteins, together with ATP, facilitate the assembly of 30S subunits in vitro. Maki et al., “The DnaK Chaperone System Facilitates 30S Ribosomal Subunit Assembly” Mol Cell 10:129-138 (2002). Interestingly, mutations in dnaK and groEL genes were shown previously to affect ribosome biogenesis in vivo. Alix et al., “Mutant DnaK Chaperones Cause Ribosome Assembly Defects In Escherichia coli” Proc Natl Acad Sci USA 90: 9725-9729 (1993); and El Hage et al., “The Chaperonin GroEL And Other Heat-Shock Proteins, Besides DnaK, Participate In Ribosome Biogenesis In Escherichia coli” Mol Gen Genet 264:796-808 (2001). It is believed that the chaperone system might be involved in the proper folding of some ribosomal proteins during 30S assembly.

During the assembly of prokaryotic 30S ribosomal subunits it is known that the small subunit proteins bind to the 16S rRNA in a hierarchical manner. Mizushima et al., “Assembly Mapping Of 30S Ribosomal Proteins From E. coli” Nature 226:214-1218 (1970); and Held et al., “Assembly Mapping Of 30S Ribosomal Proteins From Escherichia coli” J. Biol. Chem 249:3103-3111 (1974). These small subunit proteins are grouped into primary, secondary and tertiary binding proteins, depending on the requirements for prior protein binding. A primary binding ribosomal protein (S 15) binds independently to a three-way junction (3WJ) in the central domain of the 16S rRNA in the early stages of 30S subunit assembly. Binding of S15 induces a conformational change in the 3WJ formed by helices 20, 21 and 22 which leads to coaxial stacking of helices 21 and 22, while helix 20 forms a 60° angle with helix 22. Orr et al., “Protein And Mg²⁺-Induced Conformational Changes In The S15 Binding Site Of 16S Ribosomal RNA” J. Mol. Biol 275, 453-464 (1998). The stabilization of this conformation by S15 is a prerequisite for subsequent binding events during the assembly of 30S subunits in order to produce a functional 70S ribosome.

The sequence of this region of the 16S rRNA differs from the homologous sequence in human 80S ribosomes. In one embodiment, the present invention contemplates that the S15 binding site comprises a target for selective blocking of prokaryotic ribosome assembly. In one embodiment, inhibition of ribosome assembly by small-molecule compounds comprise binding to a 3WJ wherein a ribosomal conformation change is inhibited. In another embodiment the small-molecule compound directly inhibits S15 binding.

C. Non-Ribosomal Bacterial Proteins

Although several non-ribosomal proteins have been implicated in ribosome biogenesis in bacteria, none are known to be essential. For example, two DEAD box RNA helicase proteins (i.e., CsdA and SrmB) are involved in 50S biogenesis in E. coli at low temperatures. Interestingly, SrmB mutants arrest with a large ribosome subunit that is ˜40S in size which are lacking or severely reduced for several ribosomal proteins. Charollais et al., “CsdA, a cold-shock RNA helicase from Escherichia coli, is involved in the biogenesis of 50S ribosomal subunit” Nucleic Acids Res 32:2751-2759 (2004); and Charollais et al., “The DEADbox RNA helicase SrmB is involved in the assembly of 50S ribosomal subunits in Escherichia coli” Mol Microbiol 48:1253-1265 (2003). Strains harboring null mutations of CsdA or SrmB proteins, either singly or in combination, are viable at normal temperatures indicating they are not essential for ribosome biogenesis.

D. GTPase-Ribosomal Protein Structural Interaction

Precise functions for eukaryotic S. cerevisiae GTPase homologs (i.e., for example, Nog2p and Nug1p) have not been elucidated and, therefore, provide little insight into bacterial ylqF gene molecular function. In one embodiment, ylqF gene product participates directly in ribosome biogenesis, wherein ylqF gene product directly incorporates the L16 ribosomal protein into the 50S subunit. See FIG. 12. In another embodiment, a YlqF protein creates an environment whereby an L16 ribosomal protein is incorporated into a ribosomal subunit. In one embodiment, the environment comprises rRNA modification (i.e., for example, tertiary structural alteration, amine protonation, etc.). In another embodiment, the environment comprises protein modification (i.e., for example, acetylation, amidylation, phosphorylation, etc.). Although it is not necessary to understand the mechanism of an invention, it is believed that a YlqF-created environment comprises indirect mechanisms that promote ribosomal protein insertion.

The YlqF amino acid sequence structure is known and contains two distinct domains; an N-terminal GTP-binding domain and an acidic C-terminal domain separated by a conserved linker (1PUJ). Kniewel et al., “Structure of the YlqF GTPase from B. subtilis” In: New York Structural Genomics Research Consortium (2003). The YlqF protein N-terminal domain is reported as highly basic (pI of 10.12) with the first 50 amino acids prior to the GTP binding domain being rich in lysine and arginine, possibly defining a domain that would be expected to interact with rRNA. The C-terminal domain is reportedly very acidic (pI of 4.71) and has an exposed patch of negatively charged amino acids that would be expected to interact with the highly basic L16 ribosomal protein. In one embodiment, a YlqF protein co-localizes rRNA and L16. In another embodiment, YlqF protein hydrolyzes GTP to facilitate L16 insertion into the 50S subunit. In another embodiment, YlqF protein indirectly regulates an intermediary protein (i.e., for example, L35 ribosomal protein or DEAD box RNA helicase) to facilitate L16 ribosomal binding to the 45S complex.

E. GTPase Growth Promotion

Many uncharacterized bacterial GTP-binding proteins may have a role in protein translation. The present invention utilizes DNA microarray analysis of YlqF protein-depleted cells to investigate ylqF gene function. It is known that this strategy has been successfully applied to the high-throughput characterization of essential genes in yeast. Mnaimneh et al., “Exploration of essential gene functions via titratable promoter alleles” Cell 118:31-44 (2004). In one embodiment, the present invention contemplates that microarray analysis identifies ylqF gene function using YlqF protein-depleted cells by characterizing bacterial cell growth characteristics.

It is known that E. coli cells defective in ribosome assembly overproduce rRNA and ribosomal protein genes. Takebe et al., “Increased expression of ribosomal genes during inhibition of ribosome assembly in Escherichia coli” J Mol Biol 184:23-30 (1985). It is also known that the differential expression of genes in YlqF protein-depleted cells show a striking similarity to gene expression seen after bacterial cell exposure to sub-lethal concentrations of translational inhibitors. Certain translational inhibitors create an intracellular environment that is similar to what is observed during a “stringent response”. Eymann et al., “Bacillus subtilis functional genomics: global characterization of the stringent response by proteome and transcriptome analysis” J Bacteriol 184:2500-2520 (2002); compared with Sabina et al., “Interfering with different steps of protein synthesis explored by transcriptional profiling of Escherichia coli K-12” J Bacteriol 185:6158-6170 (2003).

A “stringent response” in B. subtilis has been investigated by a combination of genomic and proteomic approaches. Eymann et al., “Bacillus subtilis functional genomics: global characterization of the stringent response by proteome and transcriptome analysis” J Bacteriol 184:2500-20 (2002). “Stringent response” is a cellular mechanism that dramatically alters the expression of rRNA and many genes involved in translation in response to amino acid limitation. Although it is not necessary to understand the mechanism of an invention, it is believed that a “stringent response” is induced when an uncharged tRNA enters the A-site of the ribosome, causing the ribosome to stall. Cashel et al., “The Stringent Response” p. 1458-1496. In: F. C. Neidhardt (ed.), Escherichia coli and Salmonella typhimurium: cellular and molecular biology, vol. 2. ASM Press, Washington, D.C. (1996).

The effector molecule of a “stringent response” comprises the protein RelA that produces guanosine tetraphosphate ((p)ppGpp). Induction of the stringent response results in the strong repression of genes coding for rRNA, ribosomal proteins, and other translation factors. A YlqF protein-depletion expression profile is, for the most part, opposite of a B. subtilis RelA-dependent response; for example, genes repressed during a “stringent response” are overexpressed in YlqF protein-depleted cells. Eymann et al., “Bacillus subtilis functional genomics: global characterization of the stringent response by proteome and transcriptome analysis” J Bacteriol 184:2500-20 (2002). Further, genes overexpressed during the “stringent response” are decreased in expression in YlqF protein-depleted cells. Thus, YlqF protein-depleted cells have a gene expression phenotype similar to the “relaxed” phenotype observed in RelA mutants.

During a “stringent response”, RelA-related proteins repress the activity of both ribosomal and non-ribosomal gene products involved in the translational apparatus. In YlqF protein-depleted cells, RelA-related proteins are overexpressed (i.e., protein translation is reduced). Consequently, a ylqF gene displays a gene expression phenotype opposite of the “stringent response”; in other words, a ylqF gene product induces protein translation wherein during a “stringent response” the ylqF gene would be normally repressed. For example, when E. coli cells were treated with sub-lethal antibiotic concentrations that inhibit translational initiation or elongation, an YlqF protein-depleted expression pattern was found.

In one embodiment, yphC and yqeH gene expression is increased in YlqF protein-depleted cells. In another embodiment, yphC and yqeH gene expression promotes bacterial ribosomal biogenesis (i.e., for example, in B. subtilis). Although it is not necessary to understand the mechanism of an invention, it is believed that neither yphC nor yqeH genes are altered in expression during a “stringent response”, indicating that they are being expressed via a different mechanism. It is further believed these genes may be involved in ribosome biogenesis because their expression levels are increased when ribosome biogenesis was inhibited.

In one embodiment, the present invention contemplates that the ylaG gene is overexpressed in YlqF protein-depleted cells. In another embodiment, the ylaG gene is repressed during a “stringent response”. It is known that the YlaG E. coli protein homolog may function as a translation factor specific for the expression of Fis protein. Owens et al., “A dedicated translation factor controls the synthesis of the global regulator Fis” Embo J 23:3375-3385 (2004).

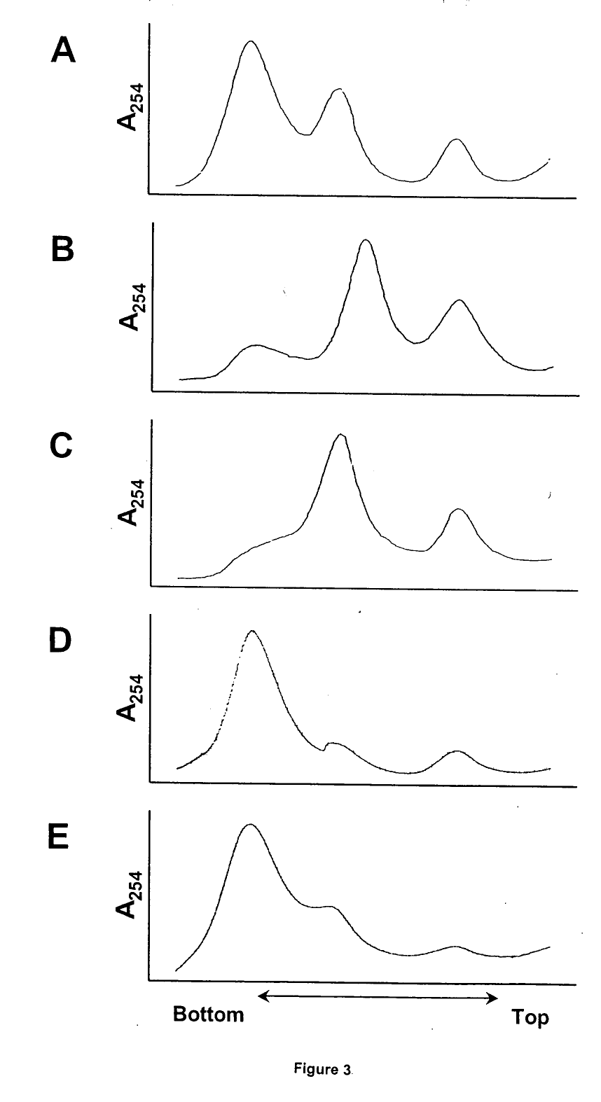

One interpretation of these results is that the RelA protein is not properly activated in YlqF protein-depleted cells. Activation of the “stringent response” by RelA protein requires a stalled, but active ribosome. Because RelA proteins are not essential, it is probable that other necessary components involved in (p)ppGpp synthesis and stalling of functional ribosomes are not being formed in YlqF protein-depleted cells. Although it is not necessary to understand the mechanism of an invention, it is believed that a “stringent response” comprises reduced functional 70S ribosome assembly or a reduced initiation of protein synthesis. Finally, several genes that were altered in expression in a RelA-independent manner (i.e., responding to translation inhibition or growth arrest) were similarly changed in YlqF protein-depleted cells. This suggests these genes are affected by lack of translation; for example, 70S ribosomes are lacking in YlqF protein-depleted cells. FIG. 3.

Global expression profiles from cells treated with translation inhibitors lend further support for a role for the ylqF gene in translation. Ribosome expression profiles from E. coli cells have been constructed using protein synthesis inhibitors that affect different steps of translation. Sabina et al., “Interfering with different steps of protein synthesis explored by transcriptional profiling of Escherichia coli K-12” J Bacteriol 185:6158-70 (2003). Of the inhibitors tested, kasugamycin treatment appears to be most similar to the response observed with YlqF protein-depleted cells.

Kasugamycin inhibits the initiation step of translation without affecting elongation. Kozak et al., “Differential inhibition of coliphage MS2 protein synthesis by ribosome-directed antibiotics” J Mol Biol 70:41-55 (1972); and Okuyama et al., “Inhibition by kasugamycin of initiation complex formation on 30S ribosomes. Biochem Biophys Res Commun 43:196-9 (1971). Consequently, the presence of kasugamycin may cause a decrease in (p)ppGpp levels. Cortay et al., “Effects of aminoglycoside antibiotics on the coupling of protein and RNA syntheses in Escherichia coli” Biochem Biophys Res Commun 112:801-8 (1983). When E. coli cells were treated with kasugamycin a dramatic increase in genes encoding the translational machinery was found (not all antibiotics tested exhibited increases in ribosomal proteins), as was the case with YlqF protein-depleted cells. As with YlqF protein-depleted cells, kasugamycin decreased expression of genes encoding proteins necessary for energy production. Although it is not necessary to understand the mechanism of an invention, it is believed that these responses are due to the cell directly upregulating the protein synthesis machinery while turning down energy production because of defects in translation.

Inhibition of protein synthesis with puromycin (which inhibits elongation) shows some overlap with both the kasugamycin and YlqF protein-depletion responses. Increased expression of ribosomal protein genes has also been noted in the response of Streptococcus pneumoniae to sub-lethal concentrations of translational inhibitors; mainly those affecting elongation. Ng et al., “Transcriptional regulation and signature patterns revealed by microarray analyses of Streptococcus pneumoniae R6 challenged with sublethal concentrations of translation inhibitors” J Bacteriol 185:359-70 (2003).

YlqF protein-depleted cells have an expression profile most similar to that observed upon inhibiting the initiation of translation. Interestingly, depletion of initiation factor 2 (IF2) in E. coli results in a relaxed phenotype highlighted by increased rRNA synthesis. Cole et al., “Feedback regulation of rRNA synthesis in Escherichia coli. Requirement for initiation factor IF2” J Mol Biol 198:383-92 (1987).

Therefore, defects in the initiation of translation, either by antibiotic treatment or depletion of a known translation initiation factor, resemble the expression profiles observed from YlqF protein-depleted cells. That these responses are not general effects of the inhibition of cell growth is noted by the failure of the protein synthesis inhibitors, mupirocin and 4-azaleucine, to show similar expression profiles. Sabina et al., “Interfering with different steps of protein synthesis explored by transcriptional profiling of Escherichia coli K-12” J Bacteriol 185:6158-70 (2003).

IV. GTPase Inhibitors

In one embodiment, the present invention contemplates mutations that reverse YlqF protein-depleted cell growth inhibition. In another embodiment, the present invention contemplates methods to identify compounds (i.e., for example, a nucleic acid or a protein) that directly interact with YlqF protein, wherein the activity of YlqF protein is inhibited.

A. Genetic Suppression

Reversing mutant phenotypes by genetic suppression has long been used to infer gene function. In one embodiment, the present invention contemplates genetically suppressed bacterial strains comprising mutations that allow cells to live in the absence of an essential GTPase by restoring the essential function performed by the essential GTPase. In one embodiment, the present invention contemplates a genetically suppressed bacterial strain expressing reduced GTPase (i.e., for example, not totally depleted of YlqF or YqeH proteins). Although it is not necessary to understand the mechanism of an invention, it is believed that this process isolates genetic mutations encoding proteins that directly interact or otherwise modulate function of the essential GTPase. In another embodiment, the present invention contemplates a genetically suppressed bacterial strain that is completely GTPase-depleted (i.e., for example, YlqF or YqeH proteins).

In one embodiment, a genetically suppressed bacterial strain comprises an overexpressed protein. In another embodiment, a genetically suppressed bacterial strain comprises multiple gene copies. It is known that genetic multicopy suppression is a type of genetic screen frequently used in bacterial genetics for isolating genes that have a functional interaction with the gene of interest. For example, a multicopy suppressor of a dominant negative allele of the era gene in E. coli has been isolated. Lu et al., “The gene for 16S rRNA methyltransferase (ksgA) functions as a multicopy suppressor for a cold-sensitive mutant of era, an essential RAS-like GTP-binding protein in Escherichia coli” J Bacteriol 180:5243-6 (1998). Additionally, Obg and EngA proteins in E. coli have been isolated as multicopy suppressors of an rrmJ gene mutation (RrmJ protein methylates 23S rRNA in E. coli). Tan et al., “Overexpression of two different GTPases rescues a null mutation in a heat-induced rRNA methyltransferase” J Bacteriol 184:2692-8 (2002).

B. Small Molecular Weight Compounds

The present invention contemplates methods to identify lead compounds (i.e., for example, small molecular weight organic molecules) for potential antibiotics, wherein high-throughput methods efficiently screen large diverse sets of compounds. In one embodiment, fluorescence assays are particularly well suited for high-throughput screening because they are sensitive, can be automated and can be rapidly performed in small volumes and large format using microtiter plates.

The present invention contemplates a three-fluorophore (i.e., markers) fluorescence resonance energy transfer (FRET) assay that allows for screening of small-molecule libraries for potential inhibitors of ribosome assembly (i.e., for example, inhibitors of YlqF or YqeH proteins). In one embodiment, a minimal 3WJ containing all determinants for S15 binding is labeled with two fluorophores (donor and acceptor 1), and a third fluorophore (acceptor 2) is attached to S15. In another embodiment, the three fluorophores are placed such that the conformational change of the 16S rRNA central domain 3WJ and the binding of S15 can be monitored simultaneously. Although it is not necessary to understand the mechanism of an invention, it is believed that a FRET assay reliably identifies compounds that bind to the junction and affect the conformation, and has the potential to identify compounds that interfere with S15 binding. Further it is believed that because of the high sensitivity and small material amounts, high-throughput assays are compatible with 384-well microtiter plates and thus can be readily adapted to high-throughput screening for novel inhibitors of 30S assembly. Klostermeier et al., “A Three-Fluorophore FRET Assay For High-Throughput Screening Of Small-Molecule Inhibitors Of Ribosome Assembly” Nucleic Acids Research 32:2707-2715 (2004).

One type of interaction between the markers that is advantageously used causes a fluorescence resonant energy transfer (FRET) which occurs when the two markers are within a distance of between about 1 angstrom (A) to about 50 A, and preferably less than about 10 A. In this case, excitation of one marker with electromagnetic radiation causes the second marker to emit electromagnetic radiation of a different wavelength that is detectable. This could be accomplished, for example, by incorporating a fluorescent marker at the N-terminal end of a protein (i.e., for example, a GTPase protein) using the E. coli initiator tRNA^{fmet}. An epitope may then incorporated near the N-terminal end (i.e., for example, SteptTag, Sigma-Genosys). Streptavidin is then conjugated using known methods with a second fluorescent marker which is chosen to efficiently undergo fluorescent energy transfer with a first marker. The efficiency of this process can be determined by calculating a Forster energy transfer radius which depends on the spectral properties of the two markers. The marker-streptavidin complex is then introduced into the translation mixture. Only when nascent protein is produced does fluorescent energy transfer between the first and second marker occur due to the specific interaction of the nascent protein StreptTag epitope with the streptavidin.

There are a variety of dyes that can be used as marker pairs in this method that will produce easily detectable signals when brought into close proximity. Previously, such dye pairs have been used, for example, to detect PCR products by hybridizing to probes labeled with a dye on one probe at the 5′-end and another at the 3′-end. The production of the PCR product brings a dye pair in close proximity causing a detectable FRET signal. In one application the dyes, fluoresein and LC 640 were utilized on two different primers (Roche Molecular Biochemicals). When the fluorescein is excited by green light (around 500 nm) that is produced by a diode laser, the LC 640 emits red fluorescent light (around 640 nm) which can be easily detected with an appropriate filter and detector. Rothschild et al., “Methods for the detection, analysis and isolation of nascent proteins” U.S. Pat. No. 6,875,592 (2005)(herein incorporated by reference).

VI. Comparative Ribosome Profiling

In one embodiment, the present invention contemplates that GTPase function may be assessed by analyzing ribosome expression profiles in GTPase-depleted bacterial cells. In one embodiment, DNA microarray analysis of YlqF protein-depleted cells was compared to previously published array data regarding translational inhibitors and a “stringent response” to determine how ylqF genes regulate ribosome expression. In one embodiment, a YlqF protein promotes ribosome expression. Other non-ribosomal GTPase proteins having a potential to regulate ribosome expression may include, but are not limited to, Era, Obg, YsxC, YloQ, or YphC. Although it is not necessary to understand the mechanism of an invention, it is believed that any compound that promotes ribosome expression, concomitantly promotes protein translation. Further, it is believed that any compound that inhibits ribosome expression, concomitantly reduces protein translation.

In one embodiment, DNA microarrays may identify genes that are overexpressed when a GTPase protein is depleted from B. subtilis cells. These genes can be converted into in vivo reporter genes induced by reductions in GTPase activity by known fusion techniques. For example, candidate genes may provide an in vivo screening method using a fluorescent (i.e., for example, a green fluorescent protein) or luminescent (i.e., for example, a luciferase protein) reporter fusion genes. In one embodiment, the fusion genes are induced when a GTPase activity is inhibited. Although it is not necessary to understand the mechanism of an invention, it is believed that small molecule compound inhibition of B. subtilis cell GTPase protein activity induces a reporter fusion gene promoter. In one embodiment, the promoter is inactive when a GTPase protein is active. In another embodiment, the promoter is active when a GTPase protein is inhibited or defective.

It has been suggested that Era, Obg, and YjeQ (YloQ in B. subtilis) proteins are involved in some aspect of protein translation. In one embodiment, the present invention contemplates that ribosome expression profiles from bacterial strains depleted of at least one GTPase may provide some indication as to whether a GTPase is involved in ribosome assembly (i.e., for example, similar to YlqF or YqeH proteins).

Depletion strains for each aforementioned GTPases may be constructed using inducible promoters including, but not limited to, an IPTG-inducible promoter and a xylose inducible such as P_{xylA}. IPTG-inducible depletion strains may be grown in the presence of 1 mM IPTG or without IPTG. Ribosomes can then be isolated by subjecting cell lysates to 10-25% sucrose centrifugation density gradients (35,000 rpm for 3.5 hours).

In one embodiment, the present invention contemplates a method comprising construction of a plurality of ribosome expression profiles from a bacterial strain comprising a Pₛₚₐₙₖ IPTG-inducible promoter. In one embodiment, the Pₛₚₐₙₖ bacterial strain is depleted in at least one GTPase protein. In one embodiment, the depleted GTPase protein is selected from the group comprising YlqF, IF2, EF-Tu, Era, Obg, YsxC, YphC, or YqeH. In one embodiment, each profile is compared to identify similarities and differences between bacterial strains. In one embodiment, the ribosome expression profiling construction comprises a B. subtilis DNA microarray. Although it is not necessary to understand the mechanism of an invention, it is believed that such information provides data regarding protein translation or ribosome biogenesis regulation. FIG. 3.

In another embodiment, the present invention contemplates a method comprising construction of ribosome expression profiles from a wild-type bacterial cell. In one embodiment, the wild-type cell expression profile is constructed in the presence of sub-lethal amounts of ribosome-specific antibiotics. In another embodiment, the wild-type bacterial cell expression profile is constructed wherein the cells are depleted of known translation factors.

In one embodiment, the present invention contemplates a ribosome expression profiling method comprising ribosomal RNA-DNA hybridization. Nucleic acid hybridization techniques are known in the art. Britton et al., “Genome-wide analysis of the stationary-phase sigma factor (sigma-H) regulon of Bacillus subtilis” J Bacteriol 184:4881-90 (2002); and Stanley et al., “Identification of catabolite repression as a physiological regulator of biofilm formation by Bacillus subtilis by use of DNA microarrays” J Bacteriol 185:1951-7 (2003). In one embodiment, hybridization comprises Cy3 and Cy5 fluorescent dyes coupled to cDNA after reverse transcription of RNA. This is in contrast to known techniques wherein the fluorescent dyes are coupled to cDNA during reverse transcription. Slides will be scanned on a GenePix 4000B scanner (Axon Instruments) and the obtained data is manipulated using GeneTraffic software (lobion). GeneTraffic performs multiple types of normalization and hierarchical clustering on the expression data. For example, differentially expressed genes may be identified by: i) iterative outlier analysis; and ii) significance analysis for microarrays (SAM) analysis. Britton et al., “Genome-wide analysis of the stationary-phase sigma factor (sigma-H) regulon of Bacillus subtilis” J Bacteriol 184:4881-90 (2002); and Tusher et al., “Significance analysis of microarrays applied to the ionizing radiation response” Proc Natl Acad Sci USA 98:5116-21 (2001). Further verification regarding changes in expression can be confirmed by real-time polymerase chain reaction analysis (RT-PCR).

In one embodiment, the present invention contemplates using ribosome expression profiles from wild-type bacterial cells grown in the presence of protein translation inhibitors as a standard reference on which to compare ribosomal profiles from GTPase-depleted strains. In one embodiment, translation inhibitors of branched chain aminoacyl-tRNA formation are selected from the group comprising 4-azoleucine and mupirocin. In another embodiment, translation inhibitors of initiation comprise kasugamycin. In another embodiment, translation inhibitors of elongation may be selected from the group comprising puromycin, chloramphenicol, tetracycline, and erythromycin. In one embodiment, the translation inhibitor concentration is an amount effective to inhibit bacterial growth 4-5 fold.

Although it is not necessary to understand the mechanism of an invention, it is believed that each translational inhibitor drug generates a specific ribosome expression profile. For example, ribosome expression profile “signatures” were identified when Streptococcus pneumoniae was treated with different translational inhibitors, each affecting different steps in translation elongation (i.e., puromycin, chloramphenicol, tetracycline, and erythromycin). Ng et al., “Transcriptional regulation and signature patterns revealed by microarray analyses of Streptococcus pneumoniae R6 challenged with sublethal concentrations of translation inhibitors” J Bacteriol 185:359-70 (2003).

In one embodiment, the present invention contemplates comparing ribosome expression profiles constructed in the presence of translational inhibitors and/or depletion of known translation factors with ribosome expression profiles of cells depleted in at least one GTPase. In one embodiment, the comparison comprises hierarchical clustering of the profile data. In another embodiment, the comparison comprises manual comparison of differentially expressed genes. Although it is not necessary to understand the mechanism of an invention, it is believed that a positive correlation between an antibiotic and/or translation factor with a GTPase expression profile suggests a possible function for the GTPase.

In one embodiment, the present invention contemplates a ribosome profiling and DNA microarray analysis comprising YlqF protein-depleted cells. In one embodiment, a comparison of YlqF protein-depleted cells with wild-type cells grown in the presence of a protein translation inhibitor identify that YlqF protein affects a late step in ribosome biogenesis. In another embodiment, a comparison of YlqF protein-depleted cells with wild-type cells grown in the presence of a protein translation inhibitor identify that YlqF protein affects protein translation initiation. In one embodiment, a ribosome expression profile comprises undetectable 70S ribosomes in Strain RB301 (i.e., for example, Pₛₚₐₙₖ -ylqF) grown without IPTG, wherein said profile is similar to cultures depleted of initiation factor 2 (Pₛₚₐₙₖ -IF2). In one embodiment, an expression profile comprises detectable 70S ribosomes in a bacterial strain depleted in elongation factor Tu protein (i.e., for example, Pₛₚₐₙₖ -EF-Tu). In one embodiment, an expression profile comprises a separation between the 50S and 30S subunits that is smaller in YlqF protein-depleted cells than in Strain RB301 cells grown in the presence of IPTG or in wild-type B. subtilis cells. Although it is not necessary to understand the mechanism of an invention, it is believed that a reduction in 70S ribosome formation is not a likely explanation because IF2-depleted cells have 50S and 30S subunits that migrate normally on sucrose gradients. It is further believed that the results suggest the peak corresponding to the SOS subunit is smaller than in wild-type cells, indicating a defect in large subunit biogenesis. In one embodiment, microarray data identifies genes that are specifically induced when ylqF gene function is inhibited. For example, by cloning a promoter for these genes behind a reporter gene, (i.e., for example, green fluorescent protein; GFP) small molecule libraries can be screened for the ability to induce these genes.

In one embodiment, the present invention contemplates that YlqF protein-depleted cells comprise an altered 50S subunit. In one embodiment, a YlqF protein acts as a chaperone to load protein(s) onto the 23S rRNA. In another embodiment, a YlqF protein assists in the folding of 23S rRNA. Alternative embodiments include, but are not limited to, methods where a YlqF protein coordinates initiation factor binding to the ribosome or acting as a sensor of nutritional status (i.e., for example, intracellular GTP levels) that signal the ribosome that conditions are sufficient to support protein translation.

VII. Comparative Genomic Analysis

In one embodiment, the present invention contemplates a method comprising a proteomic analysis of a ribosome from a cell depleted in at least one GTPase. In one embodiment, the ribosome comprises 50S subunits. In another embodiment, the ribosome comprises 30S subunits. In another embodiment, the ribosome comprises a 70S ribosome. In one embodiment, the cell comprises a depletion in YlqF protein concentration.

A. Sucrose Density Centrifugation

In one embodiment, a proteomic analysis of a bacterial cell ribosome comprises an alteration in the migration of the 50S and/or 30S subunit. Although it is not necessary to understand the mechanism of an invention, it is believed that either the 50S or 30S subunit has an aberrant composition of protein or RNA. In one embodiment, the bacterial cell comprises a YlqF protein-depleted cell. In one embodiment, the 50S and/or 30S ribosome subunits from YlqF protein-depleted cells are compared to wild-type 50S and/or 30S ribosome subunits. In one embodiment, the ribosome subunits are determined by sucrose density gradient centrifugations (10-25%) followed by agarose gel electrophoresis to confirm the presence of ribosomal nucleic acids (i.e., for example, 23S or 16S rRNA). In one embodiment, the proteomic analysis determines that a ribosomal protein is missing in a GTPase-depleted cell (i.e., for example, L16). In another embodiment, the proteomic analysis determines that a ribosome subunit is altered in size in a GTPase protein-depleted cell. In another embodiment, the proteomic analysis determines that a ribosome subunit comprises folding or conformational alterations in a GTPase protein-depleted cell.

B. Immunoprecipitation

It is known that proteins can interact with each other and can be identified by co-immunoprecipitation of the complex. Traditionally, the protein complex identification uses an antibody raised against one of the complexed proteins. In one embodiment, the present invention contemplates a method comprising antisera raised against YlqF or YqeH proteins. In another embodiment, the method further comprises immunoprecipitation of either YlqF or YqeH proteins. In one embodiment, an immunoprecipitation negative control comprises a Pₛₚₐₙₖ -ylqF or Pₛₚₐₙₖ -yqeH bacterial strain grown in the absence of IPTG.

1. Reversible Crosslinking

In order to enhance the sensitivity of immunoprecipitation a reversible crosslinking agent such as, but not limited to, dithiobis(succinimidylpropionate (DSP) or formaldehyde may be used to crosslink proteins in vivo. After immunoprecipitation, the crosslinks can be reversed by heating to yield individual proteins. In one embodiment, a cross-linked YlqF protein forms a complex with other proteins that, when isolated from cells, migrate at a size larger than its predicted molecular weight on a SDS-PAGE gel. In another embodiment, reversal of this crosslink yields a monomeric YlqF protein. Although it is not necessary to understand the mechanism of an invention, it is believed that crosslinking creates an association of a YlqF protein with itself and possibly with other proteins that can be identified by mass spectrometry.

C. Yeast 2-Hybrid Technology

The yeast 2-hybrid system is known to detect protein:protein interactions. This approach was used to identify interactions between the GTPase Obg protein and components of the general stress response in B. subtilis. Scott et al., “Obg, an essential GTP binding protein of Bacillus subtilis, is necessary for stress activation of transcription factor sigma(B)” J Bacteriol 181:4653-60 (1999). In one embodiment, the present invention contemplates a method using yeast 2-hybrid technology to identify proteins that interact with YlqF or YqeH proteins. For example, the Hybrid Hunter Yeast Two-Hybrid System® (Invitrogen) can be used and experiments performed as instructed. In one embodiment, a B. subtilis genomic library is cloned into the “prey” vector and YlqF or YqeH proteins are used as “bait”.

D. Bacterial 2-Hybrid Technology

In one embodiment, the present invention contemplates a method comprising screening for small molecule inhibitors of GTPase/ribosomal protein interactions using a bacterial 2-hybrid system.

A bacterial two hybrid system is a known signal amplification system of at least two chimeric polypeptides containing a first chimeric polypeptide corresponding to a first fragment of an enzyme and a second chimeric polypeptide corresponding to a second fragment of an enzyme or a modulating substance capable of activating said enzyme. The first fragment is fused to a molecule of interest and the second fragment or the modulating substance is fused to a target ligand. The activity of the enzyme is restored by the in vivo interaction between the molecule of interest and the target ligand. Signal amplification is generated and, for example, triggers transcriptional activation. The signal amplification system is useful in a method of selecting a molecule of interest, which is capable of binding to target ligand, wherein the interaction between the molecule of interest and the target ligand is detected with the signal amplification system. For example, a signal amplification system may comprise Escherichia coli, in which the proteins of interest are genetically fused to two complementary fragments of the catalytic domain of Bordetella pertussis adenylate cyclase. Ladant et al., “Bacterial multi-hybrid systems and applications thereof” U.S. Pat. No. 6,333,154 (2001)(herein incorporated by reference).

E. GTPase Activity Assays

In one embodiment, the present invention contemplates a method comprising the step of monitoring GTPase activity when a GTPase interacts with ribosomal subunit. In one embodiment, the GTPase is selected from the group comprising YlqF, YsxC, and YphC. In another embodiment, the GTPase comprises YqeH. In one embodiment, the ribosomal subunit is selected from the group comprises a 45S subunit, a 50S subunit, or a pre-50S subunit. In another embodiment, the ribosomal subunit comprises a mature 30S subunit or a precursor 30S subunit. In another embodiment, the ribosomal subunit comprises a ribosomal protein selected from the group comprising L16, L35, L36. In one embodiment, the ribosomal subunit comprises a ribosomal nucleic acid selected from the group consisting of 5S rRNA or 23S rRNA. In another embodiment, the ribosomal subunit comprises 16s rRNA. In one embodiment, a small molecular wieght compound inhibits the interaction.

Several assays are known which measure GTPase activity. Trahey et al., Science 238:542 (1987); and Adari et al., Science, 240:518 (1988). GTPase may be assayed in vitro, using several different techniques. One assay involves measuring the presence of GDP resulting from the hydrolysis of GTP. This assay involves combining in an appropriate physiologically buffered aqueous solution, empirically determined optimal amounts of normal cellular p21 protein, α-³²P-GTP, plus a GTPase. The solution may also contain protease inhibitors and a reducing agent. Also, since cations (i.e., for example, Mg²⁺) greatly stimulate GTPase activity they should be present in an effective amount. The reaction solution is incubated for various times and may be conducted at temperatures typically employed to perform enzymatic assays, preferably 10°-40° C., and more preferably at 37° C. At the appropriate times aliquots are removed and assayed for α-³²P-GDP. This is readily accomplished by first separating p21 protein containing bound α-³²P-GDP from the other reactants in the solution, particularly free α-³²P-GTP. This can be achieved by immunoprecipitating p21 antibodies. Immune precipitation techniques and anti-p21 antibodies are known, and routinely employed by those skilled in the art. α-³²P-GDP, is released from the immune precipitate preferably by dissolving the sample in a denaturing detergent at an elevated temperature, more preferably in 1% sodium dodecyl sulfate at 65° C. for five minutes, and chromatographing the mixture on a suitable thin layer chromatographic plate (i.e., for example, a PEI cellulose plate in 1M LiCl α-³²P-GDP is identified by its mobility relative to a known standard using suitable radiodetection techniques, preferably autoradiography. McCormick et al., “GTPase activating protein fragments” U.S. Pat. No. 5,763,573 (1998)(herein incorporated by reference).

Experimental

The following examples are intended as merely illustrative of the present invention and are not to be considered limiting.

### EXAMPLE I

### Construction of Pₛₚₐₙₖ -ylqF and Pₛₚₐₙₖ -ygeH Strains

This example describes the construction of bacterial strains that place the expression of ylqF or yqeH under the control of an isopropyl-beta-D-thiogalacto-pyranoside (IPTG) inducible, LacI repressible promoter Pₛₚₐₙₖ.

A fragment containing the ribosome binding site and the 5′ end of either the ylqF or yqeH gene was cloned into the Pₛₚₐₙₖ vector pJL86, which cannot replicate in B. subtilis. The resulting plasmids were transformed and recombined into the chromosome of B. subtilis by single crossover. This recombination places the full-length GTPase gene under the control of the Pₛₚₐₙₖ promoter.

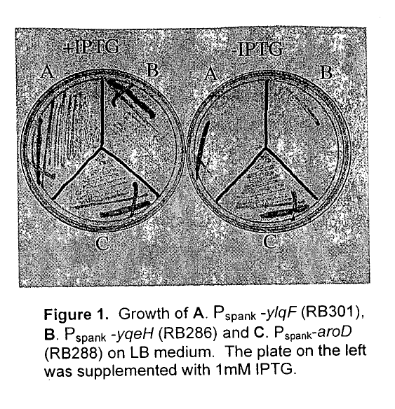

The ylqF gene is flanked by potential transcription terminators and appears to be monocistronic. Therefore, the repression of the Pₛₚₐₙₖ promoter is not expected to influence the expression of downstream genes. This was later confirmed by microarray analysis. The Pₛₚₐₙₖ -ylqF (RB301) strain was tested for IPTG-dependent growth. On LB medium containing 1 mM IPTG, RB301 formed large colonies as expected. In the absence of IPTG colonies were barely visible after 24 hours and appeared abnormal. FIG. 1.

Strain RB301 was grown initially in LB medium supplemented with 1 mM IPTG and then shifted to LB medium containing no IPTG. Growth of the culture was monitored by measurement of culture density at OD₆₀₀. In the presence of 1 mM IPTG the RB301 strain grows at a doubling time of approximately 35 min, which is indistinguishable from wild-type cells. In the absence of IPTG, a decline in the growth rate of RB301 cells is observed after about four doublings. Continued growth in the absence of IPTG shows that cells do continue growth at a very slow rate (125 min doubling time). This residual growth is likely due to a small amount of leaky expression of the Pₛₚₐₙₖ promoter.

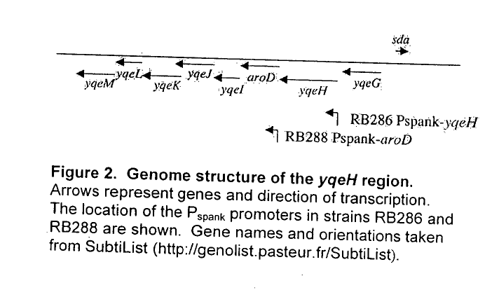

The location of the yqeH gene suggests that it is co-expressed with a number of additional genes in an operon. FIG. 2. To ensure that the Pₛₚₐₙₖ-yqeH strain would not result in a decreased expression of genes downstream of yqeH an additional strain was constructed that places aroD, the gene immediately downstream of yqeH, under the control of Pₛₚₐₙₖ. The Pₛₚₐₙₖ-yqeH and Pₛₚₐₙₖ-aroD strains were grown on LB medium in the presence of 1 mM IPTG and in the absence of IPTG. As expected, both strains formed large colonies after overnight incubation at 37° C. in the presence of 1 mM IPTG. However, only the Pₛₚₐₙₖ-yqeH strain showed reduced growth in the absence of IPTG, with very small colonies visible after 24 hours. The growth of the Pₛₚₐₙₖ-aroD strain grown in the absence of IPTG was indistinguishable from 1 mM IPTG, demonstrating that depletion of yqeH is necessary for the growth defect.

A decrease in the growth rate directly correlated with the amount of IPTG present in RB286 (Pₛₚₐₙₖ -yqeH) and RB301 (Pₛₚₐₙₖ -ylqF) cultures. Strain RB286 was grown in LB medium at 37° C. in the presence of varied amounts of IPTG. RB286 and RB301 cells grown in the presence of 1 mM IPTG grew at a rate indistinguishable from wild-type cells. Table 3. Growth rate decreased until a doubling time of 100 min was achieved in the absence of IPTG. Cells grown without IPTG still grew exponentially suggesting that leaky expression from the Pₛₚₐₙₖ promoter is occurring. Although protein levels were not measured in these cells it is believed that a reduced level of YqeH protein is responsible for the alteration in growth rate.

TABLE 3
Doubling times of Pₛₚₐₙₖ-yqeH (RB286) and Pₛₚₐₙₖ-ylqF (RB301)
strains grown in various concentrations of IPTG. Cells were incubated
in LB medium at 37° C. Under these conditions wild-type
Bacillus subtilis has a doubling time of 27 min.

| Strain/[IPTG] | 1 mM | 10 μM | 5 μM | 0 μM |
|---|---|---|---|---|
| RB286 (Pₛₚₐₙₖ-yqeH) | 28 min | 45 min | 70 min | 100 min |
| RB301 (Pₛₚₐₙₖ-ylqF) | 35 min | 70 min | 98 min | 125 min |

These results demonstrate that YqeH and YlqF protein levels are able to directly influence the growth rate of B. subtilis.

### EXAMPLE II

### Analysis of Gene Expression Changes in Cells Depleted of YlqF Protein

This example uses full-genome B. subtilis DNA microarrays to analyze the cellular gene expression response to depletion of the essential GTPase protein, YlqF. Long oligonucleotides (65mers) corresponding to all 4,106 annotated open reading frames of B. subtilis were purchased from Compugen. The oligonucleotides were resuspended in 3×SSC buffer to a final concentration of 25 μM at the Michigan State University Genomics Technology Support Facility (GTSF). Oligonucleotides were spotted onto UltraGAPS slides (Coming) using the OmniGrid (GeneMachines) robot at the GTSF. Experimental protocols for B. subtilis DNA microarrays were performed as previously described. Britton et al., “Genome-wide analysis of the stationary-phase sigma factor (sigma-H) regulon of Bacillus subtilis” J Bacteriol 184:4881-90 (2002); and Stanley et al., “Identification of catabolite repression as a physiological regulator of biofilm formation by Bacillus subtilis by use of DNA microarrays” J Bacteriol 185:1951-7 (2003).

TABLE 4
Differentially expressed genes in cells depleted of YlqF protein.

| | # of genes | # of genes |
|---|---|---|
| | overexpressed | underexpressed |
| | in YlqF protein | in YlqF protein-depleted |
| Functional class | depleted cells | cells |
| Cellular wall | 1 | 5 |
| Transport/binding proteins | 12 | 25 |
| and lipoproteins | | |
| Sporulation | 3 | 14 |
| Protein secretion | 2 | 0 |
| Carbohydrate metabolism | 4 | 55 |
| glycolysis TCA cycle | | |
| Intermediary metabolism | 7 | 64 |
| DNA replication | 0 | 2 |
| modification and repair | | |
| DNA recombination | 1 | 0 |
| RNA synthesis | 8 | 19 |
| Protein synthesis | 51 | 2 |
| Protein folding | 0 | 3 |
| Protein modification | 3 | 2 |
| Cell processes (adaptation | 3 | 48 |
| protection) | | |
| Unknown proteins | 23 | 73 |
| Electron transport chain | 0 | 7 |
| and ATP synthase | | |
| Cell division | 0 | 2 |
| Total | 118 | 321 |

Microarray analysis of YlqF protein-depleted cells showed many changes in gene expression when compared to cells in which the ylqF gene was expressed. Table 4. The ylqF gene was chosen for an initial trial experiment because it is monocistronic and depletion of this gene product will not result in the depletion of any downstream gene products. The strain containing the Pₛₚₐₙₖ-ylqF construct was grown in the presence of 1 mM IPTG or without IPTG in LB medium at 37° C. Samples for DNA microarray analysis were taken at a time when the culture lacking IPTG showed a significant slowing of growth. Conditions for growth were optimized so that this slowdown occurred at an OD₆₀₀ of 0.5-0.7. A sample from the 1 mM IPTG culture was taken at a similar OD₆₀₀ value to control for any changes in gene expression caused by increased cell density. Four independent microarray experiments were performed. Significant changes in gene expression were identified by iterative outlier analysis and Significance Analysis of Microarrays (SAM) analysis. Britton et al., “Genome-wide analysis of the stationary-phase sigma factor (sigma-H) regulon of Bacillus subtilis” J Bacteriol 184:4881-90 (2002); and Tusher et al., “Significance analysis of microarrays applied to the ionizing radiation response” Proc Natl Acad Sci USA 98:5116-21 (2001).

Cells depleted of the YlqF protein showed dramatic changes in gene expression. Over 400 genes showed a greater than two-fold change in expression. Analysis of significantly changed genes strongly indicates that cells depleted of YlqF protein are defective in translation. Ribosomal protein genes, initiation factors, elongation factors, and other proteins directly involved in translation were all overexpressed in YlqF protein-depleted cells, with the highest fold changes being 10-fold. Considering that ribosomal proteins are some of the most highly expressed proteins in the cell, a 10-fold increase in their transcripts is quite surprising. One interpretation of this result is that YlqF protein-depleted cells are not properly translating proteins and are turning up the synthesis of the translational machinery in an attempt to generate functional ribosomes. Consistent with this view is the fact that some molecular chaperones (i.e., for example, GroEL, GroES, and DnaK proteins), which assist in folding proteins, are underexpressed in YlqF protein-depleted cells. Lower amounts of protein production would be consistent with this result; less protein production results in a lower requirement for chaperones to fold these proteins. Further, genes encoding proteins involved in generating energy (glycolytic enzymes, TCA cycle enzymes, proteases) and detoxifying oxygen radicals produced by oxidative phosphorylation (catalase, superoxide dismutase, etc.) were decreased in expression. Several of the observed changes were confirmed by real-time quantitative RT-PCR (data not shown).

### EXAMPLE III

### YlqF Protein-Depleted Cells are Defective in Ribosome Assembly

This example presents data regarding ribosome expression profiling in YlqF protein-depleted cells.

Ribosome profiles of cells depleted of YlqF protein showed a defect in 70S ribosome assembly. RB301 cells were grown in the presence (1 mM) or absence of IPTG and cell lysates were analyzed by sucrose gradient density centrifugation. Daigle et al., “Studies of the interaction of Escherichia coli YjeQ with the ribosome in vitro” J Bacteriol 186:1381-7 (2004); Inoue et al., “Suppression of defective ribosome assembly in a rbfA deletion mutant by overexpression of Era, an essential GTPase in Escherichia coli” Mol Microbiol 48:1005-16 (2003); and Lin et al., “The Caulobacter crescentus CgtA(C) Protein Cosediments with the Free 50S Ribosomal Subunit” J Bacteriol 186:481-9 (2004). Cell lysates were centrifuged for 3.5 hours at 35,000 rpm through a 10-25% sucrose gradient.

A hole was made in the bottom of the centrifuge tube and profiles were obtained by continuous monitoring of A254. In the presence of 1 mM IPTG, RB301 has a ribosome profile similar to that of wild-type cells. FIGS. 3A & 3E, respectively. When grown in the absence of IPTG (125 min doubling time), the 70S ribosome peak is drastically reduced, demonstrating there are few functional 70S ribosomes assembled in YlqF protein-depleted cells. FIG. 3B. An increase in the amounts of the 30S and 50S individual subunits is also observed, consistent with a defect in 70S assembly. Additionally, a decrease in the separation between the 30S and 50S subunits was noted, suggesting there is a defect in the assembly of one of the subunits. The peak corresponding to the 50S subunits indicates a slower sedimentation rate in the YlqF protein-depleted cells than in wild-type cells. These results suggest that ylqF genes participate in translation initiation or a late step in ribosome biogenesis, possibly in the assembly of the 50S subunit.

To compare the ribosome profile of YlqF protein-depleted cells with the ribosome profile of a known translation initiation factor, a strain comprising Pₛₚₐₙₖ-infB (encoding initiation factor 2) was constructed. The resultant depletion of IF2 results in a doubling time similar to that of YlqF protein-depleted cells and a similar ribosome profile. FIG. 3C. Specifically, 70S ribosomes are not formed and the individual 30S and 50S levels are increased. However, the 30S and 50S subunits are positioned correctly in the gradient and do not show the abnormal migration as observed in YlqF protein-depleted cells. This demonstrates the reduced separation of the 50S and 30S is not due to a defect in translation initiation (or loss of 70S subunits) and that ylqF genes likely affect a process in translation distinct from IF2 protein function.

Because there are two additional genes (i.e., for example, ylxP and rbfA) downstream of infb that could also be affected by infB depletion, a control strain was constructed that placed the next gene downstream of infB under the control of the Pₛₚₐₙₖ promoter. Ribosome profiling of the Pₛₚₐₙₖ -ylxP strain showed that ribosome formation was unaffected when grown in the absence of IPTG (data not shown).

To confirm that a general defect in translation does not result in the dissolution of the 70S ribosomes under centrifugation conditions, a ribosome profile was created using cells depleted of the translation elongation factor EF-Tu (P_{spankhy}-tufA). P_{spankhy} in a mutant version of Pspank that allows higher expression in the presence of IPTG. As expected, cells depleted of EF-Tu have an increased amount of 70S ribosomes and decreased 50S and 30S subunits due to the inability to complete elongation during translation. FIG. 3D. This result shows that our observed loss of 70S ribosomes in YlqF protein- and YqeH protein-depleted cells is not due to a general defect in translation.

### EXAMPLE IV

### YlqF is Evolutionarily Related to Eukaryotic GTPases Involved in Ribosome Biogenesis

This example presents the homology relationships between prokaryotic and eukaryotic GTPase.

BLAST analysis of YlqF protein indicated an evolutionary relationship with: i) GTPases known to be involved in large subunit ribosome biogenesis, for example Nog2p and Nug1p, Bassler et al., “Identification of a 60S preribosomal particle that is closely linked to nuclear export” Mol Cell 8:517-29 (2001); and Saveanu et al., “Nog2p, a putative GTPase associated with pre-60S subunits and required for late 60S maturation steps” Embo J 20:6475-84 (2001); ii) a nucleolar GTPase that controls cellular proliferation, for example nucleostemin, Tsai et al., “A nucleolar mechanism controlling cell proliferation in stem cells and cancer cells” Genes Dev 16:2991-3003 (2002); and iii) a human nucleolar GTPase of unknown function (E-values: 6e-9 to 1e-13). The best match in the human genome is the Sprn protein (E value: 4e-36), but there is no functional information about this protein. The similarities between these proteins are not limited to the GTP binding domain as PSI-BLAST analysis using N-terminal and C-terminal sequences from YlqF protein that did not include the GTP-binding domain also identified significant similarities to these eukaryotic GTPases. This analysis provides additional evidence that YlqF protein plays a role in ribosome biogenesis in bacteria. The precise functions of Nog2p and Nug1p in ribosome biogenesis are still unknown.

### EXAMPLE V

### YqeH Protein-Depleted Cells Have an Altered Ratio of 23S:16S rRNA

This example presents an analysis of ribosomal RNA in YqeH protein-depleted cells.

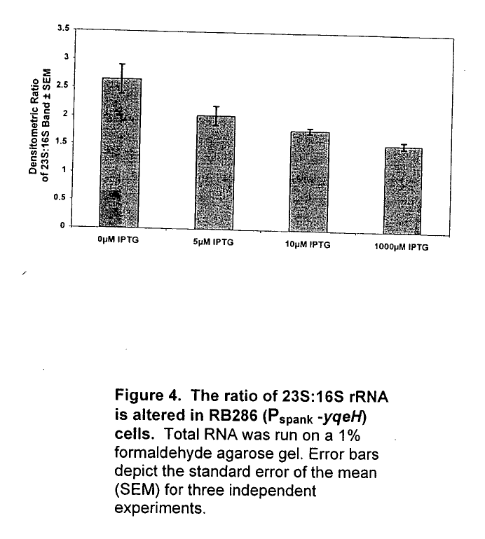

Isolated RNA from YqeH protein-depleted cells reveal that the 16S rRNA band was much fainter than the 16S rRNA band in RNA isolated from wild-type cells or YlqF protein-depleted cells. The ratios of 23S:16S rRNA in RNA isolations from pSPANK-yqeH cells grown in 1 mM IPTG, 10 μM IPTG, 5 μM IPTG, or no IPTG were determined. RNA isolated from cells grown in 1 mM IPTG and wild-type cells had a 23S:16S rRNA ratio of approximately 1.5. RNA isolated from cells depleted of YqeH protein showed an increased ratio of 23S:16S rRNA. FIG. 4. The increasing 23S:16S ratio correlated with the amount of IPTG added to the culture. Decreasing the amount of IPTG added to the culture (slower growth rate) resulted in higher 23S:16S rRNA ratios. In cultures grown in the absence of IPTG (100 min doubling time), the 23S:16S ratio was ˜2.6. If we assume that the levels of 23S rRNA are not altered in these cells then the 16S rRNA is 42% less abundant in cells depleted of YqeH protein.

The decrease in 16S rRNA correlates well with growth rate; i.e., as growth rate decreases the amount of 16S rRNA decreases. A 50% decrease in 16S rRNA is quite dramatic considering the rRNA is one of the most abundant molecules in the cell. These results strongly suggest a role for the yqeH gene in translation, specifically a role in 16S rRNA maturation or stability. The addition of a ribonuclease inhibitor to cells prior to RNA isolation had no effect on the increased ratio suggesting that 16S rRNA is not being degraded during isolation.

### EXAMPLE VI

### YqeH Protein-Depleted Cells are Defective in 30S Subunit Assembly

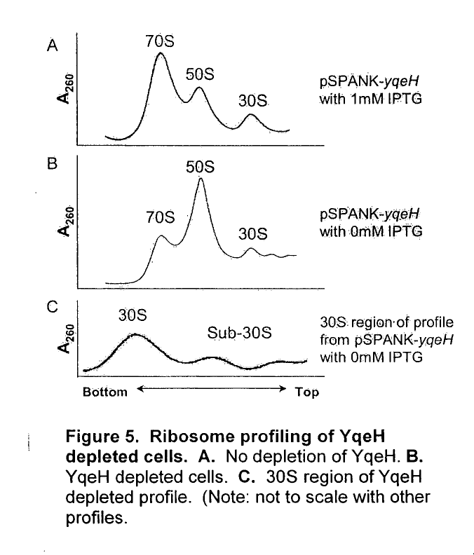

This example presents data showing the 30S subunit assembly is defective in YqeH protein-depleted cells. FIG. 5.

The ribosome profiles were constructed from RB286 cells grown in the presence (1 mM) or absence of IPTG. Cells lysates were prepared and centrifuged over a 10-25% sucrose gradient. In the presence of 1 mM IPTG (FIG. 5A) RB286 cells show a distribution of 70S ribosomes and individual 50S and 30S subunits that is similar to wild-type cells (wild-type not shown). When grown in the absence of IPTG (FIG. 5B) there is a striking decrease in the 30S subunit peak and loss of 70S ribosomes. The 50S peak appears largely unaffected. An RB288 control strain that depletes the expression of genes downstream of the yqeH gene had a normal ribosome profile. (Data not shown). The RB286 depletion profile indicates that 16S rRNA is specifically depleted in YqeH protein-depleted cells. FIG. 5B. Further, a small peak that is slightly smaller than the 30S subunit is believed to be an accumulating intermediate during 30S subunit biogenesis in YqeH protein-depleted cells. FIG. 5C. These data indicate that the biogenesis of the 30S subunit is defective.

### EXAMPLE VII

### Purification of YlqF-His₆ and YqeH-His₆ Proteins

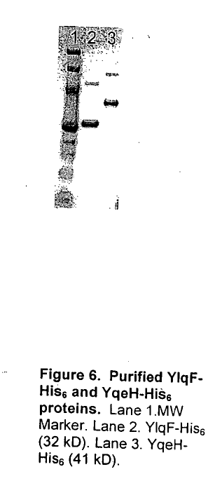

His₆ fusions to the C-terminus of both YlqF and YqeH proteins were constructed, overexpressed in E. coli, and purified using Ni beads as per manufacturer's protocol (Novagen). FIG. 6. The purified proteins may be used in generating polyclonal antibodies. Both YlqF-His₆ and YqeH-His₆ proteins are functional in vivo as determined by creating strains in which the only copy of either essential GTPase was the fusion protein. The phenotype of these strains was indistinguishable from that of wild-type cells, indicating the fusions are functional.

### EXAMPLE VIII

### Creating Strains to be Used for the Isolation of Suppressors of the Growth Defect in YlqF- or YqeH Protein-Depleted Cells

Described below are strains that have been developed for use in suppressor analysis of the ylqF and yqeH genes.

(A) ylqF Gene

The pSWEET vector comprises a xylose-inducible promoter that can be used to isolate suppressors for both the ylqF and yqeH genes by expressing genes cloned into this plasmid. The ylqF gene was cloned downstream of the bgaB gene in the vector pSWEET-bgaB, a vector that contains a heat stable version of the lacZ (bgaB) gene driven by the xylA promoter. Bhavsar et al., “Development and characterization of a xylose dependent system for expression of cloned genes in Bacillus subtilis: conditional complementation of a teichoic acid mutant” Appl Environ Microbiol 67:403-10 (2001).

Expression from the xylA promoter is xylose inducible/XylR repressible and has been optimized for controlled regulation of gene expression in B. subtilis. The presence of the bgaB gene in the construct allows for detection of mutations affecting the P_{xylA} promoter, which are undesirable. The additional advantage of this system over the Pₛₚₐₙₖ system is that there are two copies of the xylR gene (encoding the xylose repressor); therefore very few suppressors will be mutations affecting xylR. The vector also contains sequences that allow the insertion of the P_{xylA}-bgaB-ylqF construct at the amyE locus in the chromosome.

After inserting the xylose inducible ylqF gene into B. subtilis at amyE, the ylqF gene at its native locus was completely deleted and replaced by an antibiotic resistance gene (erm) using a PCR mediated knockout strategy.

The resulting strain (RB353) is now xylose-dependent for optimal growth on LB medium, although the doubling time of this strain is approximately 45 min (as opposed to 125 min for RB301 grown without IPTG), suggesting some significant expression of the ylqF gene in the absence of xylose. However, without xylose only small colonies are present after 24 hours and suppressors allowing fast growth were easily isolated following mutagenesis with 0.6% ethyl methanesulfonate (EMS). Without mutagenesis no fast growing suppressors were observed.

Twenty-five suppressors that formed healthy colonies of wild-type appearance in 24 hours were isolated. All twenty-five suppressors were white on X-Gal containing medium, indicating that suppression was not due to mutations affecting the activity of the Pxyl promoter. To confirm that the suppressor mutations were not linked to either the P_{xylA}-bgaB-ylqF (cmr) or ΔylqF::erm marker both of these markers were back-crossed from the suppressor strain into a wild-type background and assayed for xylose independent growth. All of the suppressors were unable to form healthy colonies in the absence of xylose and appeared similar to RB353, clearly indicating that an extragenic mutation unlinked to either marker was responsible for suppression.

(B) yqeH Gene

A similar strategy has been employed for isolating suppressors of the yqeH gene. The yqeH gene was cloned into the pSWEET vector, inserted at amyE, and the native copy of the yqeH gene was replaced with a spectinomycin (spc) marker to create strain RB406. This strain does not include bgaB as was the case for the ylqF strain described above. The resulting strain is dependent on xylose for growth. Suppressor mutations isolated in a fully repressed background (no xylose) are believed to bypass the essential function that the yqeH gene provides to the cell. Results using Strain RB406 suggest that such mutations may be difficult or impossible to isolate. By growing the P_{xylA}-yqeH strain in a concentration of xylose that allows partial growth, it is possible to isolate mutations in genes encoding proteins that directly interact with the yqeH gene or otherwise modulate the function of the yqeH gene. By providing the cell with a lowered amount of YqeH protein (i.e., YqeH protein-depleted) it should be possible to isolate mutations that alter the function of an interacting protein, which would not be possible in a fully depleted situation.

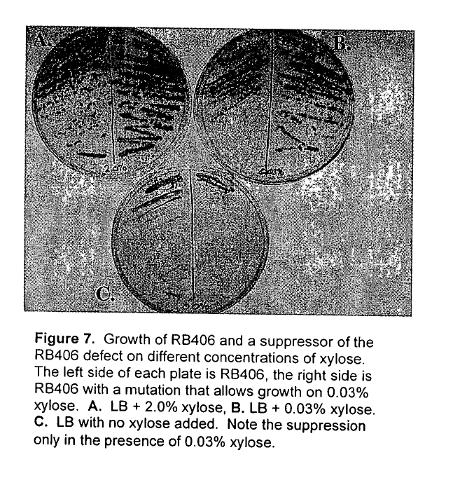

A culture of RB406 was mutagenized with 0.6% EMS and cells were plated on LB medium with 0.03% xylose overnight at 37° C. RB406 has a doubling time of approximately 45 min under these conditions (versus 25 min for growth in 2.0% xylose) and mutants that display wild-type growth are easily distinguished from non-suppressed cells on plates after 24 hours of growth. Without mutagenesis no spontaneous suppressors were found. Eighteen (18) suppressors were isolated, of which all are capable of wild-type growth on 0.03% xylose. Fourteen (14) suppressors are not able to suppress the growth defect of RB406 cells grown without xylose, demonstrating that some expression of YqeH protein is necessary for suppression. See FIG. 7. To confirm that the suppressor mutations were not linked to either the P_{xylA}-yqeH or ΔyqeH::spc markers, both were backcrossed from the suppressor strains into a wild-type background. Sixteen of the eighteen backcrosses were unable to form healthy colonies on LB plates containing 0.03% xylose, demonstrating that the suppressor mutations are not linked to either marker and are extragenic. The other two suppressors are likely mutations resulting in the overexpression of YqeH protein due to a mutation in the P_{xylA} promoter.

### EXAMPLE IX

### Construction of Strains to Deplete Five Additional Essential GTPases

This example analyzes the gene expression profiles of strains that are depleted for an essential GTPase.

DNA microarray analysis of the expression profiles of cells depleted of YlqF protein indicates that the ylqF gene participates in translation, perhaps in initiation. In order to characterize other essential GTPases of B. subtilis using DNA microarrays, Pₛₚₐₙₖ -GTPase gene fusions using era, obg, ysxC, and yphC were constructed. Each of these fusions produced similar results to the Pₛₚₐₙₖ-ylqF and Pₛₚₐₙₖ-yqeH gene fusions. All strains form small colonies when plated on LB medium without IPTG and show a dramatic reduction in growth rate when grown in liquid medium without IPTG.

A putative rho-independent transcription terminator immediately follows the ysxC and obg genes and fusing the Pₛₚₐₙₖ promoter to these genes at their native locus will not result in the depletion of genes downstream. The yphC gene does have an additional gene (i.e., for example, gpsA) immediately downstream. As with the yqeH gene fusion, a Pₛₚₐₙₖ fusion to the gene immediately downstream of the yphC gene was constructed. Depletion of the GpsA protein had no effect on growth.

### EXAMPLE X

### Construction of a YlqF-GFP Fusion Protein

B. subtilis is particularly amenable to GFP gene fusion because any gene in the B. subtilis genome can be generated in a single step. A ylqF-gfp gene fusion construct has been created.

The ylqF gene was fused to gfpmut2 by cloning a 3′ fragment of the ylqF in frame with gfp (downstream of ylqf). Cormack et al., “FACS-optimized mutants of the green fluorescent protein (GFP)” Gene 173:33-8 (1996). This construct was transformed into competent B. subtilis and the ylqF-gfp gene fusion was shown to be functional since transformants were unable to be isolated.

The ylqF-gfp fusion will be visualized in live cells using a Nikon microscope equipped with an ORCA CCD camera. Exponentially growing cells will be harvested and placed on agarose pads according to previously described protocols. Britton et al., “Characterization of a prokaryotic SMC protein involved in chromosome partitioning” Genes Dev 12:1254-9 (1998). The localization of the fusion protein will be determined.

Although it is not necessary to understand the mechanism of an invention, it is believed that if YlqF protein is associated with ribosomes, a localization pattern similar to that observed for ribosomal proteins in B. subtilis would be expected. Lewis et al., “Compartmentalization of transcription and translation in Bacillus subtilis” Embo J 19:710-8 (2000).

Alternatively, localization of YlqF protein by immunofluorescence using polyclonal antibodies generated against the proteins YlqF or to GFP may be performed. Due to artifacts associated with fixation of cells for immunofluorescence and the ease of visualizing GFP fusions, GFP experiments are done first using immunofluorescence as a backup strategy.

### EXAMPLE XI

### Functional Analysis of the 30S Subunit in YlqF Protein-Depleted Cells

Binding of mRNA and fMet-tRNA to the 30S subunit are early steps in the process of translation initiation. If YlqF protein-depleted cells are defective in initiation, either because the 50S subunit is not properly formed or because the YlqF protein is an integral part of the initiation step, then the status of the 30S subunit with respect to binding mRNA or initiator tRNA may be affected. The experiments outlined below will address if there are significant differences in the amount of initiator tRNA or mRNA bound to the 30S ribosome. These results will indicate if the 30S subunit is compromised in its ability to initiate translation.

To address the level of mRNA bound to 30S subunits a strain in which the bgaB gene can be expressed at high levels in a background in which YlqF protein can be depleted was constructed. The RB301 strain was transformed with the pSWEET-bgaB plasmid, creating a strain in which the bgaB gene can be induced at high levels in the RB301 background by the addition of xylose. This strain will be grown in the presence or absence of IPTG with 2.0% xylose to induce bgaB gene expression. 30S subunits will be isolated by sucrose density gradient centrifugation and the levels of bgaB gene associated with the 30S subunit will be determined by real-time RT-PCR. Differences in the level of mRNA associated with the 30S subunit between YlqF⁺ and YlqF protein-depleted cells would further support a conclusion that translation initiation is defective.

If YlqF protein participates in the association of mRNA with the 30S ribosome then YlqF protein-depleted cells will have 30S subunits containing less bgaB mRNA. If YlqF protein-depleted cells have a defect in 50S subunit assembly and lack of this assembly inhibits 70S ribosome formation the 30S initiation complex should not be affected. Under these conditions, more bgaB mRNA would be associated with 30S subunits from YlqF protein-depleted cells.

A similar experiment will be done with fMet-tRNA, analyzing the levels of this tRNA bound to the 30S subunit by Northern hybridization. Similar logic applies to interpreting the results for this experiment. For example, more initiator tRNA bound to 30S subunits from ylqF⁺ cells versus YlqF protein-depleted cells, suggests that initiation is not ready to take place in ylqF⁻ cells. Conversely, more fMet-tRNA bound to the 30S ribosome in YlqF protein-depleted cells. Consequently, the defect observed in YlqF protein-depleted cells is likely due to a 50S subunit defect.

### EXAMPLE XI

### YlqF Protein Rescue of 70S Ribosome Formation in vitro

The goal of this example is to determine if YlqF protein can rescue the ribosome biogenesis defect observed in YlqF protein-depleted cells.

YlqF protein-depleted cells may be unable to initiate translation due to loss of YlqF protein activity. Altered mobility of the 50S subunit suggests YlqF protein participates in the assembly of the large subunit; however, because after depletion of YlqF protein for several generations, it is possible that the observed defect is an indirect result of YlqF protein depletion. A successful rescue of the ribosome assembly defect in vitro will support a direct role for YlqF protein in ribosome biogenesis.

RB301 cells will be grown in the absence of IPTG under conditions in which little or no 70S ribosomes are formed and cell lysates prepared. Purified YlqF-His₆ protein will be added to the YlqF protein-depleted lysates and the sample will be subjected to sucrose gradient density centrifugation after selected periods of incubation in order to determine if YlqF protein supplied in vitro can restore the 50S subunit to wild-type size and lead to the formation of 70S ribosomes.

YlqF-His₆ protein is functional in vivo since cell viability is supported when YlqF -His₆ protein is the only form of YlqF protein expressed in the cell. Although reaction conditions including buffers, RNA stabilizing reagents, and GTP and/or trace metal concentrations will need to be optimized empirically, successful rescue of the depleted phenotype would establish a direct role of the ylqF gene in ribosome biogenesis.

### EXAMPLE XII

### Synthesis of DNA, RNA, and Protein in YlqF Protein-Depleted Cells

Cells depleted of YlqF protein show a dramatic reduction in growth rate. Because growth rate was determined by accumulation of mass as measured by an increase in optical density, YlqF protein-depleted populations make protein more slowly than wild-type cells.

To quantitate the decrease in protein synthesis we will measure amino acid incorporation in cells depleted of YlqF protein will be determined. RB301 (Pₛₚₐₙₖ-ylqF) cells will be grown in the presence of 1 mM IPTG. Cells will be transferred into medium containing no IPTG and the incorporation of 35S-methionine into total protein will be assayed at several timepoints during depletion of YlqF protein. DNA and RNA synthesis will be measured in YlqF-depleted cells by determining the incorporation of ³H-thymidine and ³H-uridine into DNA and RNA, respectively. Because YlqF protein participates in translation it is expected that protein synthesis will slow down first, followed by alterations in RNA and DNA synthesis.

### EXAMPLE XIII

### Analysis of rRNA Processing Using Stable RNA DNA Microarrays

The level of 16S rRNA in YqeH protein-depleted cells is lower than in wild-type cells, suggesting a defect in processing of this molecule. Normally, the status of rRNA processing is followed by Northern hybridizations using multiple probes to mature and immature regions of rRNA. A single hybridization using DNA microarrays corresponding to mature and immature regions of rRNA can greatly reduce the time and number of experiments needed to characterize possible processing defects. A similar approach has been successful in analyzing processing of yeast stable RNA molecules. Peng et al., “A panoramic view of yeast noncoding RNA processing” Cell 113:919-33 (2003).

Oligonucleotides (40-50mers) will be produced that correspond to mature 16S, 23S and 5S RNA. Additional probes to sequences present in the primary 30S RNA transcript will be constructed to detect unprocessed messages and processing intermediates. The processing events that occur in B. subtilis to produce the mature rRNAs are not well understood, therefore the events that occur in the processing of E. coli rRNA will be used as a guide in probe design. Li et al., “RNase G (CafA protein) and RNase E are both required for the 5′ maturation of 16S ribosomal RNA” Embo J 18:2878-85 (1999).

Oligonucleotides will be chosen so that the melting temperatures of every oligonucleotide is similar, specificity is ensured, and secondary structure is minimized (this latter point may be difficult to achieve for many of the transcripts as stable RNAs form extensive secondary structures). Control oligonucleotides that have no sequence similarity to the oligos in this array will be constructed and used as positive (doped in RNA for labeling and hybridization controls) and negative controls (non-specific hybridization control).

The rRNA microarray will be used to determine if YqeH protein depletion alters the processing or production of rRNA. For example, Pₛₚₐₙₖ-yqeH (RB286) cells will be grown in 1 mM IPTG or in the absence of IPTG (YqeH protein-depleted). Cells will be grown in the absence of IPTG until RB286 reaches a doubling time of 100 min. This will indicate the cells have been fully depleted of YqeH protein. Cells will be harvested and RNA isolated. Hybridizations will be performed twice with the fluorescent dyes reversed to control dye bias in the experiments. Slides will be imaged using a GenePix 4000B scanner (Axon). Data storage and preliminary analysis will be performed using GeneTraffic (Iobion) software.

The results will show that the ratio of 23S:16S rRNA is altered in YqeH protein-depleted cells. Visual inspection of the RNA indicates that the ratio is different due to a decrease in the amount of 16S rRNA. This decrease reflects a defect either in 16S rRNA processing or in the assembly of the 30S subunit. Because the 23S and 16S rRNA genes are transcribed as a single 30S transcript that is then processed into precursor 23S and 16S fragments, YqeH protein is not directly involved in the synthesis of 16S rRNA.

A strain mutated for the rnc gene, which encodes for RNAseIII, will be used as a positive control. rnc gene mutants accumulate 30S precursor RNA in E. coli and B. subtilis. Herskovitz et al., “Endoribonuclease RNase III is essential in Bacillus subtilis” Mol Microbiol 38:1027-33 (2000). Microarray analysis will confirm the abnormal ratio of 23S:16S rRNA previously detected on agarose gels.

Single hybridization techniques can also analyze processing defects. Defects in ribosomal RNA processing/modification or ribosome assembly can result in the accumulation of partially processed rRNA intermediates. Mature 16S rRNA is processed during ribosome assembly, with the final maturation step occurring very late in assembly, possibly after initiation starts. 30S subunits assembled with immature 16S rRNA are not functional, leading to speculation that processing of the pre-16S rRNA and the control of translation are intimately coupled. Srivastava et al., “Mechanism and regulation of bacterial ribosomal RNA processing” Annu Rev Microbiol 44:105-29 (1990). Identification of a precursor 16S rRNA in YqeH protein-depleted cells would lend further support to a role for yqeH genes in translation.

Alternatively, Northern blots can be used to detect rRNA transcripts of abnormal size by using probes to the mature 16S or 23S rRNA. Additional probes that correspond to sequences removed from the precursor fragments will also be tested. The drawback of the Northern blot approach is that several experiments need to be conducted to obtain the amount of information we can obtain from a single microarray experiment. However, the Northern approach has been successfully used in the past to address rRNA processing in bacteria. If altered transcripts are detected we will localize the 5′ ends of the transcripts by primer extension.

### EXAMPLE XIV

### YqeH Protein Binding to 16S rRNA in vivo and in vitro

This example determines if YqeH protein interacts with RNA. Initial attempts will be performed in vivo because the RNA species to which YqeH protein binds will not be a mature 16S rRNA. Also, if YqeH protein recognizes 16S rRNA only in the context of the ribosome, an in vitro approach may not work.

Defects in 16S rRNA accumulation suggests that YqeH protein may directly interact with 16S rRNA or a precursor. A potential zinc ribbon motif (i.e., for example, CXXCN₂₅CXXC) has been identified that is absolutely conserved in all of the YqeH protein homologs found to date, are found in several ribosomal proteins and may play a role in RNA:protein interactions. Laity et al., “Zinc finger proteins: new insights into structural and functional diversity” Curr Opin Struct Biol 11:39-46 (2001).

Analysis of protein:DNA interactions was previously optimized for use in B. subtilis. Lindow et al., “Structural maintenance of chromosomes protein of Bacillus subtilis affects supercoiling in vivo” J Bacteriol 184:5317-22 (2002); and Quisel et al., “Control of sporulation gene expression in Bacillus subtilis by the chromosome partitioning proteins Soj (ParA) and Spo0J (ParB)” J Bacteriol 182:3446-51 (2000). The ability of a YqeH protein to bind 16S rRNA precursor or mature 16S rRNA in vivo will be assessed by crosslinking cellular components with formaldehyde and immunoprecipitating YqeH protein using YqeH specific antibodies. Formaldehyde will form stable protein:protein, protein:DNA, and protein:RNA crosslinks.

After immunoprecipitating the YqeH protein, the crosslinks will be reversed by incubation at 37° C. Samples will be treated with DNAse to remove any DNA present; subsequently the DNAse will be inactivated by heat. The presence of 16S rRNA will be determined by RT-PCR. Specific bands for 16S rRNA will be confirmed to exclude contaminating RNA species. Mock immunoprecipitations will be performed as a control to ensure that any observed interactions are due to isolation of crosslinked YqeH protein:RNA species.

Alternatively, in vitro analysis of YqeH protein binding to rRNA will be performed using purified YqeH -His₆ protein. YqeH -His₆ protein has been expressed in the cell as the only form of YqeH protein and cells grew normally; demonstrating that the fusion protein is functional in vivo. Interactions between YqeH-His₆ protein and 16S rRNA will be assessed by affinity chromatography using biotinylated rRNA immobilized to a column. Variables such a zinc, GTP, and GDP concentrations will be varied to identify optimal conditions for observing an interaction.

These experiments will include mutational analysis of the CXXC motifs within YqeH proteins and deletion analysis of the RNA to determine what part of 16S rRNA is bound by YqeH proteins. If YqeH -His₆ protein is capable of binding and hydrolyzing GTP then at the least the GTP binding domain is likely to be folded properly. Alternatively, immobilization of YqeH -His₆ protein to Ni resin can determine if rRNA is bound to YqeH after incubation with cell lysate. Optimal conditions for observing significant binding will need to be determined empirically.

### EXAMPLE XV

### Analysis of Proteins in the 30S and Sub-30S Peaks

Depletion of YqeH protein results in a decreased amount of 30S subunits and the appearance of a small peak slightly smaller than the 30S peak. This peak may represent a small subunit assembly intermediate, possibly an intermediate similar to the 21S reconstitution intermediate (RI) observed during in vitro assembly of the 30S subunit. Culver et al., “Efficient reconstitution of functional Escherichia coli 30S ribosomal subunits from a complete set of recombinant small subunit ribosomal proteins” RNA 5:832-43 (1999); and Held et al., “Rate determining step in the reconstitution of Escherichia coli 30S ribosomal subunits” Biochemistry 12:3273-81 (1973). Determining the identity of the proteins and RNA associated with this peak may identify potential defects in 30S assembly in YqeH protein-depleted cells.

To determine the status of ribosomal RNA in the sub-30S peak cell lysates from YqeH protein-depleted cells are run over 10-25% sucrose centrifugation density gradients and 200 μl fractions are collected across the gradient. Samples will be run on 1% TAE formaldehyde agarose gels and stained with ethidium bromide to determine which RNA species correspond with the 30S peak. Detection of 23S and 16S bands in fractions corresponding to the 50S and 30S peaks are easily performed.

Fractions corresponding to the sub-30S peak will be collected, pooled and concentrated. The same will be done with a mature 30S peak from non-depleted cells. The concentrated fractions will be run on a 15% SDS-PAGE gel to identify which, if any, of the small subunit proteins are missing from the sub-30S peak. Differentially associated proteins will be identified by mass spectrometry. If this peak represents a biogenesis intermediate similar to the 21S RI, observed during in vitro E. coli 30S subunit assembly, then missing proteins from this intermediate will be identified. In E. coli these proteins are known as the tertiary binding proteins S2, S3, S10, S14, and S21. As an alternative approach we will analyze the sub-30S and mature 30S subunits by LC-MS/MS.

### EXAMPLE XVI

### Isolation of Dominant Negative Mutants for the Analysis of YqeH Protein Function

The present example analyzes dominant-negative mutations to assess YqeH protein function.

If the YqeH protein functions as a 16S rRNA processing factor a dominant-negative mutation could create a protein that binds but does not cleave the RNA. In YqeH protein-depleted cells this RNA may be unstable and degraded, whereas in a yqeH gene dominant-negative mutant, this RNA may be stabilized. Missense mutations in the yeast ribosomal protein rps14 gene are known to determine its role in 18S ribosomal processing. Antunez de Mayolo et al., “Interactions of yeast ribosomal protein rpS14 with RNA” J Mol Biol 333:697-709 (2003); and Jakovljevic et al., “The carboxy-terminal extension of yeast ribosomal protein S14 is necessary for maturation of 43S preribosomes” Mol Cell 14:331-42 (2004). Null mutations in the rps14 gene did not show an increase in precursor but instead showed processing intermediates that were smaller than 18S. Only when missense mutations were used that still produced protein were they able to isolate 20S precursor rRNA at high levels, indicating a role for the rps14 gene in rRNA processing.

The yqeH gene was cloned into the pSWEET vector for xylose dependent expression in B. subtilis. The pSWEET -yqeH plasmid will be mutagenized by amplifying the plasmid in XL1-RED cells, an E. coli strain that lacks DNA repair functions. Chusacultanachai et al., “Random mutagenesis strategies for construction of large and diverse clone libraries of mutated DNA fragments” Methods Mol Biol 270:319-34 (2004); and Farrow et al., “Identification of essential residues in the Erm(B) rRNA methyltransferase of Clostridium perfringens” Antimicrob Agents Chemother 46:1253-61 (2002). This strain has been used in numerous studies for generating libraries of random mutations in many studies.

A second procedure for mutating yqeH DNA uses error-prone PCR. Mutated plasmids will be linearized (to ensure marker replacement of amyE) and transformed into wild-type B. subtilis. Transformants will be replica plated onto media containing 2.0% xylose and colonies that no longer have the ability to grow in the presence of xylose (expressing mutant YqeH protein) will be isolated. Positive clones will be backcrossed and tested again to confirm lethality in the presence of xylose. The yqeH gene will be sequenced to identify the mutation(s) potentially responsible for the dominant-negative phenotype. The 23S:16S ratio, 16S rRNA processing, and ribosome profiles of dominant negative mutants will be performed. Alternatively, the mutagenized library can be transformed into a strain that lacks the native copy of the yqeH gene in an attempt to identify temperature sensitive mutations in the yqeH gene. These would also be useful in investigating YqeH protein function.

### EXAMPLE XVII

### Genetic Suppressor Analysis of ylqF and yqeH Genes

This example describes a strategy for mapping yqeH gene suppressors. Mapping of the ylqF gene suppressors uses the same strategy and will not be repeated.

A B. subtilis strain was constructed in which the slow growth of cells partially depleted of YqeH protein can be suppressed by extragenic mutations. Following the isolation of a number of extragenic suppressors, the genes responsible for suppression of the growth defect are identified.

Briefly, individual suppressor strains will be transformed with the pIC333 (tet) plasmid and mini-Tn10 libraries will be constructed as described. Petit et al., “Tn10-derived transposons active in Bacillus subtilis” J Bacteriol 172:6736-40 (1990). This plasmid normally contains a spectinomycin (spc) resistance gene. An original strain used to perform the suppressor analysis of YqeH protein-depleted cells is already resistant to spectinomycin, consequently the spc gene may be replaced with a tetracycline (tet) resistance gene.

Approximately 15,000 individual colonies will be selected and combined for each library. Chromosomal DNA will be isolated and then backcrossed into RB406 on media containing 0.03% xylose and tetracycline. Any colonies that are tetracycline resistant and are able to form wild-type appearing colonies on 0.03% xylose are candidates for having the mini-Tn10 element linked to the suppressor mutation. To confirm linkage of the mini-Tn10 element to the suppressor mutation chromosomal DNA will be isolated from colonies that are both tetracycline resistant and able to form colonies on 0.03% xylose. DNA can then be backcrossed into RB406 onto tetracycline, 2.0% xylose LB plates. Individual colonies will be then plated on LB tet 0.03% xylose and the linkage of the mini-Tn10 element will be determined. These transformations will be done with low quantities of DNA to minimize congression of unlinked markers.

Any linkage above 5% (maximum observed congression frequency) is considered positive linkage to the suppressor mutation. Although linkage by transformation of up to 50 kB has been demonstrated in B. subtilis, it is more likely that a linked element will be within 10-20 kB of the suppressor mutation (closer if the linkage is quite high). To determine where linked mini-Tn10 elements are located the elements and flanking chromosomal DNA will be isolated using standard procedures. The mini-Tn10 element contains an origin of replication in E. coli and chromosomal DNA surrounding the transposon can be easily cloned in E. coli. Primers specific to the mini-Tn10 element are then used to sequence the flanking chromosomal DNA. Genes located near the element will be identified by analyzing the chromosomal region on Subtilist. Individual genes in the region will be subjected to complementation testing to determine which gene is responsible for suppression.

Mutation identification will provide information regarding the function of YqeH and YlqF proteins. The present screening method involves cells only partially depleted of these GTPase proteins, consequently other proteins that directly interact with YlqF or YqeH proteins may be detected.

### EXAMPLE XVIII

### Identifying Suppressors of YlqF or YqeH Protein-Depleted Cells

This example describes a library of the B. subtilis chromosome cloned behind the P_{xylA} promoter to identify genes that, when overexpressed, suppress the growth defect of cells depleted of YlqF or YqeH proteins. This library was previously used to identify the zapA gene as one overexpression suppressor of minD overexpression. Gueiros-Filho et al., “A widely conserved bacterial cell division protein that promotes assembly of the tubulin-like protein FtsZ” Genes Dev 16:2544-56 (2002).

The library will be transformed into RB286 (Pₛₚₐₙₖ -yqeH) or RB301 (Pₛₚₐₙₖ -ylqF) cells. Individual colonies will be tested for their ability to grow in the absence of IPTG (decreased GTPase expression) and in the presence of 2.0% xylose (induction of library expression). Transformants that are able to grow under these conditions will be isolated and the fragment of DNA driven by PxylA expression will be sequenced. If more than one complete gene is isolated on the fragment, each gene will be tested for suppression individually. Experiments will then be performed to characterize the mechanism of suppression by the overexpressed gene.

### EXAMPLE XIX

### Expression Profiling and Proteonomic Analysis

Growth Conditions

All Examples were performed at 37° C. in Luria-Bertani (LB) medium. Antibiotics were added at the following concentrations, when necessary: chloramphenicol 5 μg/ml and spectinomycin, 100 μg/ml, tetracycline 12.5 μg/ml. Isopropyl-beta-D-thiogalactopyranoside (IPTG) was purchased from Teknova.

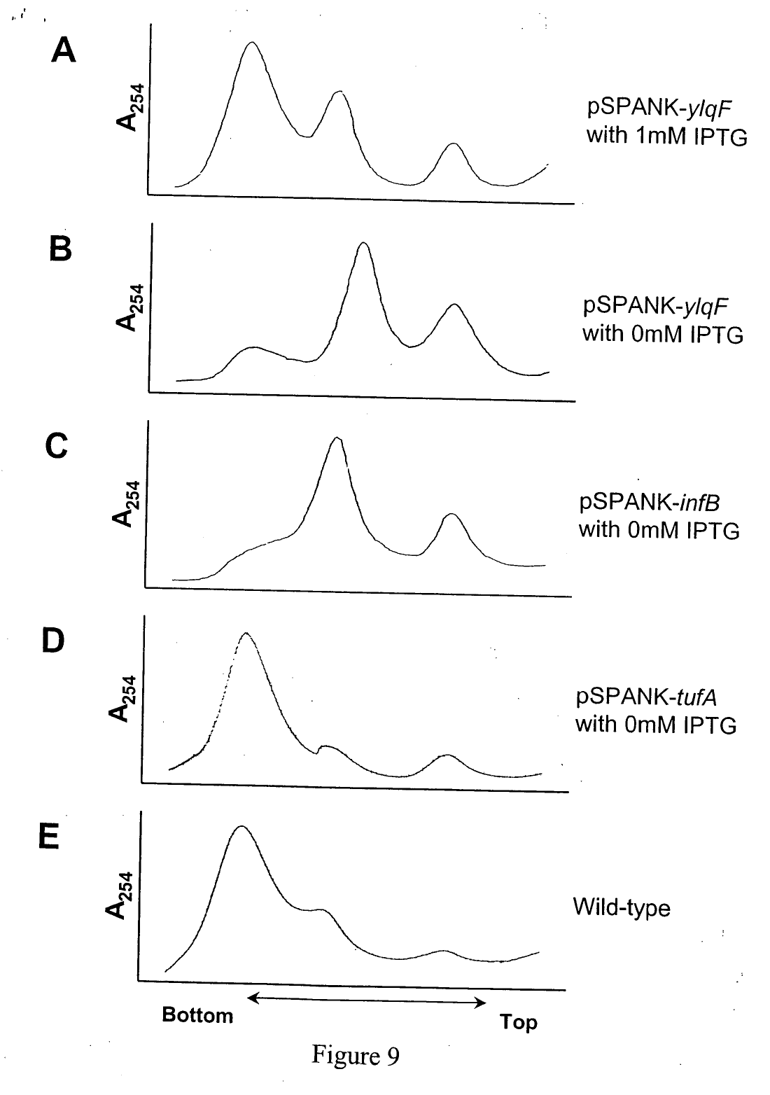

The growth rate of RB301 (Pₛₚₐₙₖ-ylqF) is dependent on the concentration of IPTG added to the medium; there appears to be an inverse correlation between the amount of YlqF protein present in the cell and the doubling time of the culture. RB301 grown in the presence of 1 mM IPTG (full induction) has a doubling time similar to wild-type cells grown in LB medium at 37° (25 min). This contrasts with a doubling time that is six times slower (150 min) when RB301 is grown in the absence of IPTG. These cells are viable indicating that the limited growth supported without IPTG is presumably due to leaky expression from the Pₛₚₐₙₖ promoter. Intermediate rates of growth were supported by different concentrations of IPTG, the more IPTG provided to the cell correlated with faster growth rates. FIG. 9. These results suggest that YlqF protein facilitates a rate-limiting step in a process that governs growth rate.

Strain Construction

All strains were derived from the wild-type strain RB247 (JH642) that is trp-, phe-. Strain RB301 (Pₛₚₐₙₖ-ylqF) was created by cloning a 217 bp PCR product containing the 30 bp upstream of the start codon of the ylqF gene to 187 bp downstream of the start codon into the Pₛₚₐₙₖ plasmid pJL86, creating pLS19. pLS19 was transformed into RB247, and single crossover recombination results in the placement of the full length ylqF gene behind the IPTG inducible, LacI repressible Pₛₚₐₙₖ promoter.

RB418 (Pₛₚₐₙₖ -infB) was created by cloning a 230 bp fragment of infB into pJL86 creating plasmid pLS34.

RB440 (P_{spankhy} -tufA) was created by cloning a 217 bp fragment of tufA into pJL87, which contains a more active version of the Pₛₚₐₙₖ promoter P_{spankhy} -, creating plasmid pWU3.

The more active promoter was necessary because fully activated Pₛₚₐₙₖ was unable to produce high enough levels of EF-Tu protein to support wild-type growth. The genome sequence reveals that ylqF and tufA genes are flanked on their 3′ end by putative transcription terminators, suggesting that no other gene will be depleted in these strains. However, the infB gene is followed immediately by the ylxP gene.

To control for possible downstream effects of regulation of the infB gene, and to more specifically assign a phenotype, a Pₛₚₐₙₖ -ylxP strain was created. RB420 (Pₛₚₐₙₖ-ylxP) was created by cloning a 203 bp fragment of gene ylxP into pJL86 creating plasmid pLS35.

Single crossover recombinations of all of these constructs into RB247, selecting for chloramphenicol resistance, result in the genes of interest being placed under the control of the inducible promoter. Strain RB395 was constructed by cloning the ylqF gene into the pSWEET plasmid under the control of the P_{xylA} promoter. This construct was linearized and inserted into the amyE locus in RB247 creating strain RB393 (data not shown).

Chloramphenicol resistant colonies were confirmed to have lost the ability to degrade starch, indicating disruption of the amyE gene. The entire native ylqF gene was deleted by long flanking homology PCR in which we replaced the ylqF gene coding sequence with an erythromycin resistance gene.

Deletion of the ylqF gene was performed in the RB393 background grown in the presence of 2% xylose. The resulting strain was xylose dependent for growth; colonies were unable to form on LB medium in the absence of xylose.

DNA Microarray Analysis

RB301 was grown in LB medium in the presence of 1 mM IPTG to an OD₆₀₀ of 0.5. Cells were pelleted, washed twice with LB medium, and then diluted back into pre-warmed LB medium containing 1 mM IPTG or lacking IPTG. Cells were grown up until a significant change in the growth rate was observed in the culture without IPTG.

Samples for microarray analysis were taken at similar OD₆₀₀ values to minimize cell density effects on gene expression. Four milliliters of culture was added to an equal volume of −20° C. methanol to immediately cease bacterial metabolism and stabilize the RNA. Total RNA was extracted using the RNeasy mini kit (Qiagen). Quality and amount of the RNA was assessed using a Nanodrop ND-1000 spectrophotometer and by 1% agarose gel electrophoresis. In all cases the 260/280 ratio was near 2.0 and there was no sign of degradation of the RNA by visual inspection. An indirect method of generating labeled cDNA and array hybridization conditions were performed. Long oligonucleotides corresponding to each gene in the B. subtilis genome were purchased from Compugen. These oligonucleotides were resuspended in 3×SSC at a concentration of 25 μM and spotted onto Corning UltraGAPS-II slides at the Michigan State Genomics Technology Support Facility.

Slides were scanned on an Axon 4000B microarray scanner and visualized using GenePix Pro 4.1 software (Axon). Scanning was performed in such a way as to capture the same amount of information in both the Cy3 and Cy5 channels. Four independent biological replicates were performed. Data was exported into Excel® and normalized based on making the total intensities of both channels equal after processing out genes that were not expressed. Spots that did not have 75% of their pixels have a value that was 1SD above background were eliminated from the analysis unless that gene was significantly expressed in the other sample, in which case the background value was assigned. This allowed a ratio to be generated and is expected to underestimate fold changes in these cases (actual value would be lower than background). Statistically significant changes in gene expression were determined by iterative outlier analysis. Britton et al., “Genome-wide analysis of the stationary phase sigma factor (sigma-H) regulon of Bacillus subtilis” J Bacteriol 184:4881-4890 (2002).

Four iterations were performed. Gene expression analysis was subsequently confirmed for several genes by quantitative real-time RT-PCR. (Syber Green/Machine® Kit). Genes that were confirmed by this method are shown in Table 5.

TABLE 5
Classes of genes differentially expressed in cells depleted of ylqF.

| | # of genes | # of genes |
|---|---|---|
| | overexpressed | underexpressed |
| | in YlqF | in YlqF |
| | protein-depleted | protein-depleted |
| Gene Classification^{A} | cells | cells |
| Metabolism of | 4 | 39 |
| carbohydrates and related | | |
| structures | | |
| Metabolism of amino acids | 1 | 32 |
| and related structures | | |
| Intermediary metabolism - | 2 | 15 |
| others | | |
| Adaptation to | 5 | 22 |
| stress/detoxification | | |
| Transport | 11 | 22 |
| RNA synthesis | 10 | 9 |
| Protein folding | 0 | 2 |
| Protein synthesis | 51 | 0 |
| Unknown | 24 | 36 |

^{A}Gene classifications as annotated in Subtilist.

Microarray analysis of cells depleted of YlqF protein suggested a role for the ylqF gene in translation. The observed changes in gene expression in cells depleted of YlqF protein suggest potential functions for this GTPase. Therefore, oligonucleotide DNA microarrays corresponding to the four thousand one hundred six (4,106) protein coding genes of B. subtilis were used to probe changes in gene expression. Parallel cultures of RB301 (pSPANK-ylqF) were grown in LB medium at 37° in the presence of 1 mM IPTG. After reaching balanced growth one culture was washed and resuspended in LB medium lacking IPTG. Cells were allowed to grow until a significant decrease in doubling time was observed, usually after 5-7 generations. Samples for RNA isolation were taken from both cultures at similar OD₆₀₀ readings to minimize gene expression changes due to differences in cell density.

Expression profiles comparing YlqF protein-depleted cells with cells expressing YlqF protein suggest a role for the ylqF gene in translation. Many ribosomal proteins, translation factors, and other proteins involved in translation were upregulated in cells depleted of YlqF protein. In some cases, individual ribosomal proteins were more highly expressed by as much as 10-fold, which is surprising considering that ribosomal protein genes are among the most highly expressed in the cell during rapid growth. The large number of proteins involved in translation that were increased in expression in cells depleted of YlqF protein suggested that protein synthesis was abnormal. Proteins involved in general metabolism, adaptation to stress, and protein folding were the major classes of underexpressed proteins in the cell.

A decrease in the expression of genes involved in generating energy and detoxifying cellular stresses associated with energy production is also consistent with decreased translation since protein synthesis is the most energy consuming process in the cell. In further support of a role for the ylqF genein translation, the YlqF protein-depleted expression profiles are similar to the transcriptional response of Escherichia coli cells treated with sublethal concentration of antibiotics affecting translation. Sabina et al., “Interfering with different steps of protein synthesis explored by transcriptional profiling of Escherichia coli K-12” J Bacteriol 185:6158-6170 (2003).

Microscopy

RB301 cells were grown in the presence of 1 mM IPTG or grown without IPTG until a doubling time of 150 minutes was achieved. Microscopy was performed essentially as previously described. Britton et al., “Characterization of a prokaryotic SMC protein involved in chromosome partitioning” Genes Dev 12:1254-1259 (1998).

The nucleoid morphology of YlqF protein-depleted cells is consistent with inhibition of translation. Cells treated with certain antibiotics that inhibit translation exhibit a chromosome partitioning defective phenotype in which the chromosome greatly compacts near the center of the cell. The antibiotic chloramphenicol, which binds to the 50S subunit of the ribosome and affects elongation and fidelity, has been shown to cause fusion of segregated chromosomes in the center of the cell. van Helvoort et al., “Chloramphenicol causes fusion of separated nucleoids in Escherichia coli K-12 cells and filaments” J Bacteriol 178:4289-4293 (1996).

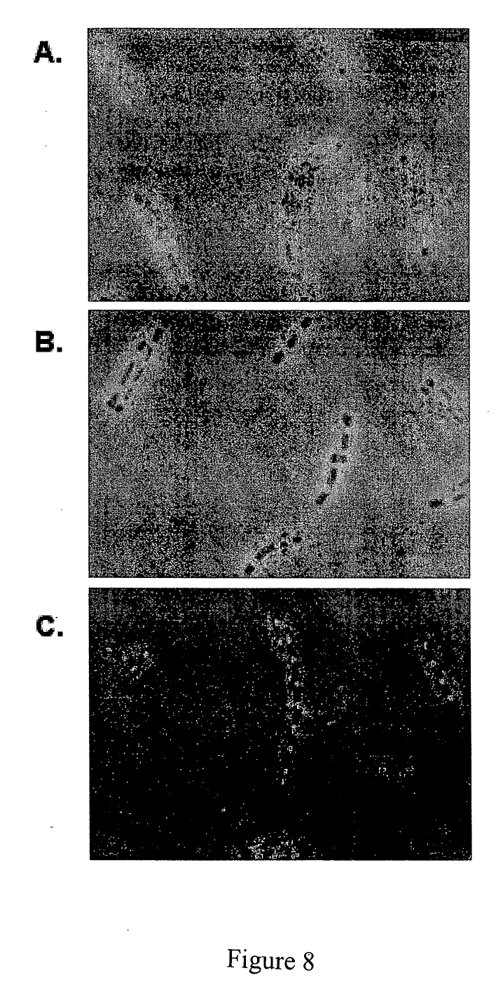

The cellular morphology and nucleoid phenotype of cells depleted of YlqF protein were analyzed found that these cells have a strikingly similar appearance to cells inhibited for translation. Cells depleted of YlqF protein contained circular nucleoids that were tightly condensed in the center of the cell with large regions of the cell devoid of DNA. FIG. 8B. A similar, but less pronounced, condensation of the nucleoid was previously noted in YlqF protein-depleted cells. Morimoto et al., “Six GTP-binding proteins of the Era/Obg family are essential for cell growth in Bacillus subtilis” Microbiology 148:3539-3552 (2002). A nucleoid phenotype similar to YlqF protein-depleted cells was observed when cells were treated with the antibiotic tetracycline at levels that inhibited cell growth. FIG. 8C. This is in contrast to RB301 cells grown in the presence of IPTG in which many of the nucleoids have a bi-lobed structure appearance that is normally observed in wild-type cells. FIG. 8A. These results further support a role for the ylqF gene in translation.

Ribosome Profiles

Ribosome profiles were prepared by sucrose density centrifugation of lysates of indicated cells grown to OD₆₀₀ of 0.5. Charollais et al., “The DEADbox RNA helicase SrmB is involved in the assembly of 50S ribosomal subunits in Escherichia coli” Mol Microbiol 48:1253-1265 (2003); and Lin et al., “The Caulobacter crescentus CgtAC protein cosediments with the free 50S ribosomal subunit” J Bacteriol 186:481-489 (2004).

Sucrose density gradients were prepared by discontinuous loading of multiple density layers and allowing diffusion overnight for a continuous gradient. Sucrose layers were prepared in 10 mM Tris-HCl buffer (pH 7.5) with 10 mM MgCl₂, 50 mM NH₄Cl, and 1 mM DTT. The strains were cultured in LB medium at 37° containing the indicated concentration of inducer for several generations to deplete the cells of YlqF protein until a constant growth rate was observed. Cells were grown to an OD₆₀₀ of 0.5 in 150 mL cultures. Chloramphenicol (Sigma) was added to a final concentration of 100 μg/mL five minutes prior to harvesting to prevent ribosome runoff. Cells were pelleted and resuspended in 6 mL of lysis buffer consisting of 10 mM Tris-HCl (pH 7.5), 60 mM KCl, 10 mM MgCl₂, 0.5% Sodium Deoxycholate, 0.5% Tween 20, 1 mM DTT, 1× Complete EDTA-Free Protease Inhibitors (Roche), and 10 U/mL RNase-free DNase (Roche). Cells were lysed by French press, and lysates were clarified by centrifugation at 16,000 g for 20 min. Lysates were loaded on top of prepared gradients and centrifuged using an SW41 rotor (Beckman) for 3.5 h at 35,000 RPM. After centrifugation, the bottom of the gradient was punctured and the gradient was drawn out and monitored for UV absorbance using a flow cell.

70S ribosome formation and 50S subunit biogenesis are defective in YlqF protein-depleted cells, and consequently result in a protein translation defect. One possible explanation for the translation defect in YlqF protein-depleted cells is that 70S ribosomes are not properly assembled. Ribosome assembly in YlqF protein-depleted cells were analyzed using ribosome profiles by sucrose gradient density centrifugation.

Pₛₚₐₙₖ-ylqF cells were grown in the presence of 1 mM IPTG and in the absence of IPTG. RB301 cells grown in the presence of 1 mM IPTG had a ribosome profile indistinguishable from wild-type cells. FIG. 9A. However, two defects are apparent from the ribosome profiles of cells depleted of YlqF protein. FIG. 9B. First, there was a severe reduction of 70S ribosomes in YlqF protein-depleted cells, consistent with the sharp decrease in growth rate of these cells. Correspondingly, levels of individual 50S and 30S ribosomal subunits were increased in YlqF protein-depleted cells. Second, the 50S subunit did not sediment normally. Alignment of the profiles demonstrated that the large subunit migrated abnormally through the sucrose gradient and sedimented at 45S rather than 50S. This slower migration of the 45S subunit suggests the large subunit is either lacking protein components from the subunit, has a smaller version of the 23S rRNA or is missing 5S rRNA, and/or is in a less compact conformation. The 23S rRNA isolated from YlqF protein-depleted cells was normal in size, although small changes would not have been detected (data not shown).

To confirm these results, a second strain was constructed capable of depleting YlqF protein. The expression of the ylqF gene was placed under the control of a xylose inducible promoter (P_{xylA}) at an ectopic locus (amyE) and then deleted the normal copy of the ylqF gene from its native locus by marker replacement. The consequent depletion of YlqF protein from this strain also yielded a reduction in 70S ribosomes and the abnormal 45S large subunit, confirming the phenotype of YlqF protein depletion in ribosome biogenesis (data not shown).

The ribosome profile of YlqF protein-depleted cells is distinct from profiles of cells depleted of IF2 or EF-Tu proteins. Strains were created that would deplete either IF2 (expressed by the infB gene) or EF-Tu (expressed by the tufA gene), both of which are also GTPase proteins. RB418 (PₛₚₐₙₖinfB) or RB440 (P_{spankhy}-tufA) were grown in levels of IPTG that result in a generation time of ˜150 min, similar to YlqF protein-depleted cells. Cell lysates were prepared and ribosome profiles were generated. FIGS. 9C & 9D, respectively. RB418 yielded a profile that one would expect from cells depleted of an initiation factor; i.e., greatly reduced levels of 70S ribosomes and an increase in 50S and 30S subunits.

Significantly, the migration of the 50S subunit was normal, indicating that the 45S peak observed in YlqF protein-depleted cells is not due simply to defective translation initiation. RB440 cells had a profile that was expected for a defect in an elongation factor; i.e., ribosomes appeared to be locked in their 70S form with a modest reduction in 50S and 30S subunits in the cell. FIG. 9D. These results demonstrate that the ribosome assembly defects observed in YlqF protein-depleted cells is not merely due to inhibition of translation or to an indirect effect of slow growth. Further, the data suggest that the levels of 70S ribosomes are dependent on the amount of YlqF protein in the cell.

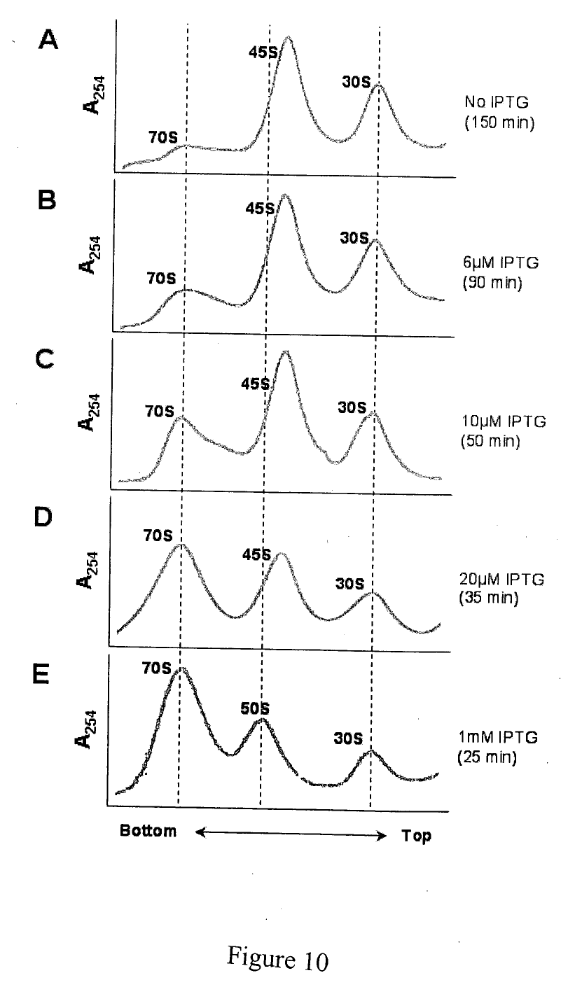

To test if YlqF protein directly influences the level of functional 70S ribosomes, RB301 (Pₛₚₐₙₖ-ylqF) was incubated with varied concentrations of IPTG and analyzed ribosome profiles under each condition. The results demonstrate that 70S ribosome formation correlates with the levels of YlqF protein synthesized in the cell. FIG. 10. When RB301 cells were grown in the presence of 1 mM IPTG (generation time 25 min) cells displayed a ribosome profile similar to that observed in wild-type cells (FIG. 10E). When grown in the absence of IPTG (generation time 150 minutes) RB301 cells were devoid of 70S ribosomes. FIG. 10A. When RB301 cells were grown using intermediate concentrations of IPTG (supporting generation times of 40, 50, and 90 min) a correlation was observed between the formation of 70S ribosomes and generation time. FIG. 10B-D. As the growth rate became faster, the proportion of 70S ribosomes increased. Although it is not necessary to understand the mechanism of an invention, it is believed that the faster growth rate is a result of increased YlqF protein levels in response to increased IPTG concentration.

The primary defect in YlqF protein-depleted cells is abnormal biogenesis of the 50S ribosomal subunit. When cells were depleted of YlqF protein the 50S ribosomal subunit migrated abnormally at 45S, suggesting that proteins normally associated with the 50S subunit were lacking or that the large subunit was less compact. The migration of the 50S subunit was analyzed in RB301 cells grown using intermediate concentrations of IPTG to determine if the 45S peak was still present at faster growth rates. Interestingly, even at doubling times approaching wild-type growth, the 45S ribosomal subunit was still present. As the concentration of IPTG added to the cells increased, the 45S level decreased with a corresponding increase in functional 70S ribosomes and growth rate.

Increased 50S production from the 45S intermediate may occur as more YlqF protein is produced in the cell. These newly formed active SOS subunits, as well as recycled 50S subunits, are immediately coupled with the available 30S subunits to form 70S ribosomes. Consequently, there is not a shift from a 45S intermediate towards a SOS subunit as growth rate increases. These results strongly suggest the defect in YlqF protein-depleted cells is the inability to form functional 50S subunits.

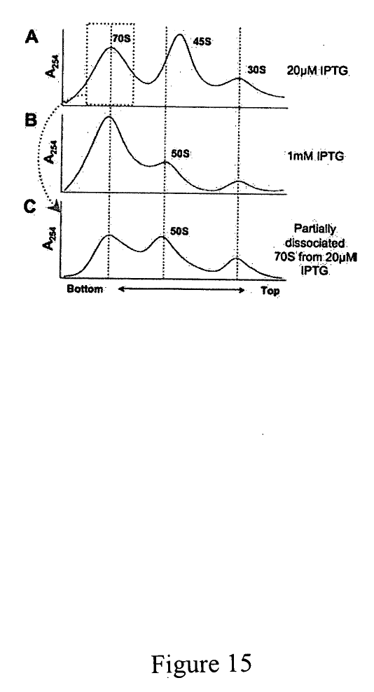

In support of this model, 70S ribosomes formed using intermediate concentrations of IPTG consist of mature 50S and 30S subunits. A 70S peak was isolated from a 20 μM IPTG (doubling time 42 minutes) ribosome profile. See FIG. 15A. The 70S peak was dissociated into subunits using low concentrations of Mg²⁺ at an incubation temperature of 37° C. See FIG. 15C. Ribosome profiling of the partially dissociated 70S particles showed that the large subunit is a SOS subunit and not a 45S subunit (compare with 50S subunit form wild-types cells in FIG. 15B). 45S subunits incubated under these conditions are stable and would have been detected if they were present in the 70S complex (data not shown). Therefore, many of the large ribosomal subunits in YlqF-depleted cells are 50S in size even though they are not observed as free subunits in the sucrose gradient profiles. Although it is not necessary to understand the mechanism of an invention, it is believed that the defect in YlqF-depleted cells represents an inability to form mature 50S subunits.

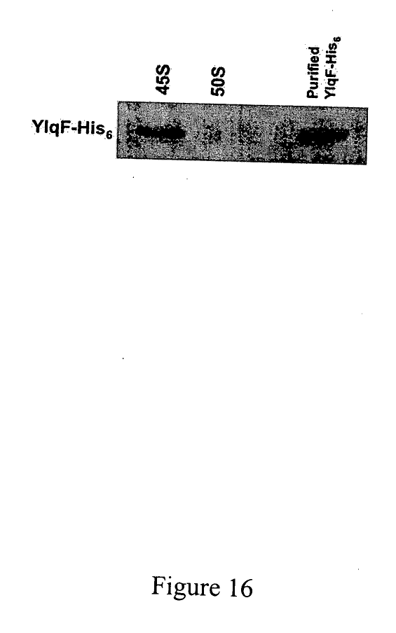

Unlike the mature SOS subunit, the 45S intermediate subunit is believed to interact with YlqF in vitro. A B. subtilis strain was constructed that expresses a YlqF-His₆ fusion protein as the only functional YlqF copy in the cell. This B. subtilis strain was viable and grew at the same rate as a wild-type strain indicating that the fusion protein is functional. The YlqF-His₆ fusion protein was purified and tested for association with purified 45S intermediates and mature 50S subunits. YlqF-His6 and either the 45S intermediate or 50S subunits were mixed and incubated for 10 minutes at 37° C. The mixtures were then fractionated on 10-25% sucrose gradients and individual fractions corresponding to the 45S and 50S peaks were collected. Western blot analysis using polyclonal antibodies raised against YlqF demonstrated that YlqF was able to bind the 45S intermediate but not the 50S subunit in vitro. See FIG. 16. Other studies have shown that YlqF-His6 does not migrate at 45S on its own (data not shown). Although it is not necessary to understand the mechanism of an invention, it is believed that YlqF functions as a factor involved in the biogenesis of the 50S subunit.

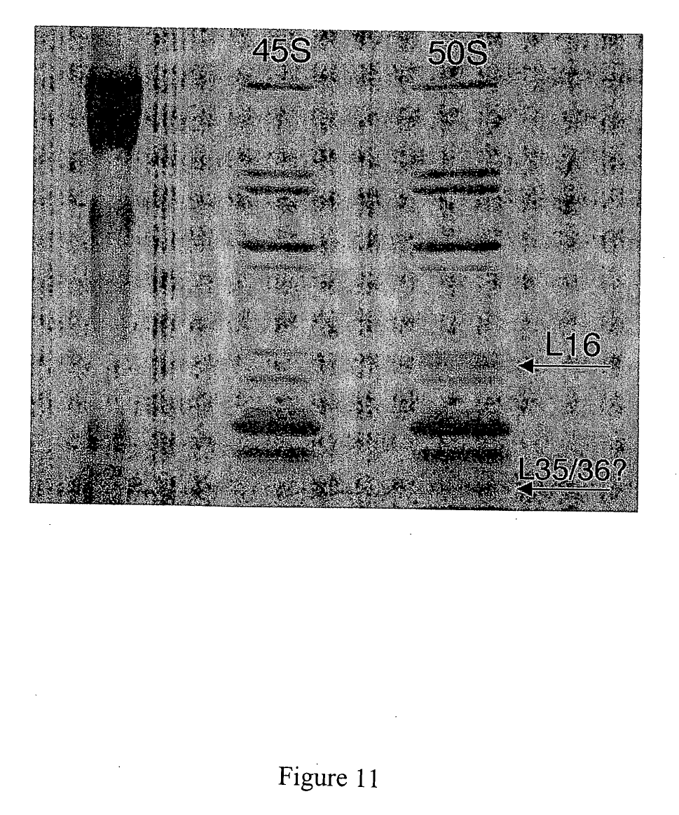

Ribosomal protein L16 is missing or greatly reduced in the 45S particle. FIG. 11. A 45S ribosome assembly intermediate profile was constructed by isolating fractions corresponding to the 45S peak from YlqF protein-depleted cells, and determining the proteins present. These profiles were compared to the protein composition of the 50S subunit from IF2 protein-depleted cells. The 50S subunit from IF2 protein-depleted cells was used because it migrates the same as the wild-type 50S subunit, the 50S subunit will be free of contaminating 70S subunits, and the cells will be doubling at the same rate as YlqF protein-depleted cells.

Isolated fractions from the 50S and 45S fractions were precipitated and run on a 12% SDS-PAGE gel. FIG. 11A. A single band migrating at 16 kD was lacking in three independent fractions of the 45S subunit. Subsequent analysis of this protein by mass spectrometry identified this protein as L16. In the lower molecular weight region of the gel, the 45S particle was consistently less intense when compared to the 50S particle. The data suggest that both the L35 and/or L36 are underrepresented (data not shown).

50S subunits from RB301 cells grown in the presence of 1 mM IPTG were found to display a wild-type ribosome profile. Analysis of the 50S subunit proteins on a 12% SDS/PAGE gel yielded a protein distribution indistinguishable from IF2 protein-depleted cells (data not shown).

Proteomic Analysis

Protein samples were reduced and alkylated and then digested with sequencing grade porcine trypsin (Promega) and analyzed by nano-scale LC/MS/MS using a Surveyor HPLC system connected to a ThermoElectron LTQ-FT. Briefly, digested peptides were trapped on a 100 μm by 5 mm nano-trap packed with Magic C18AQ 5 μm packing material (Michrom Bioresources). After the peptides were trapped, they were eluted with a gradient of 2%B to 40%B in 25 minutes (40 minutes total analysis time per sample) (A=0.1% formic acid, B=100% acetonitrile+0.1% formic acid) on a 75 um×100 mm picrofrit column (New Objectives) packed with Magic C18AQ.

Mass spectra were acquired with the following instrument parameters; the top 8 ions were isolated and analyzed with the FT detector (1-2 ppm accuracies) while simultaneously being fragmented in the LTQ to obtain MS/MS data (200-400 ppm accuracies). Peak lists were extracted using extract_msn and searched against the B. subtilis sequences from the NCBI bacterial genome collection using the xΔtandem search algorithm (Beavis Informatics, CA). Identifications were considered correct if the protein score had a probability score of −3.0 or below. False positive identification rates were determined by reversing the B. subtilis database and using xtandems modified Probit algorithm. When protein identifications were based on a (p) of −2.0 or below (used in this study), estimated false positives and reversed sequence false positives were 2 for the reverse database search and 0 for the false positive modeling calculation.

To confirm the ribosomal expression results a nano-scale LC/MS/MS of the entire protein content was performed of the 45S intermediate and 50S subunit. The results demonstrated that ribosomal protein L16 was easily detected in the 50S subunit but was not significantly detected in the 45S subunit, confirming the 1D-gel data. In the 50S subunit fraction ribosomal protein L16 was identified by 5 unique peptides covering 42% of the protein with a log(expect) score of −3.0 or below (total log(expect)=−26.5). In addition, L16 had a Log(I) score of 5.51. The Log(I) score is calculated by the xTandem® software and is the sum of the fragment ion intensities for the entire protein which can be viewed as a rough indicator of abundance. In the 45S subunit L16 was identified by only one peptide and had a total log(expect)=−2.8 with log(I) score of 3.31.

Bioinformatic Analysis.

BLAST and PSI-BLAST analyses were performed at the NCBI website (ncbi.nlm.nih.gov/BLAST/). The default parameters were used for BLAST analysis. For PSI-BLAST analysis proteins identified as having an e value of <0.005 were included for further iterations. Three iterations were performed.

### EXAMPLE XX

### Correlating YlqF Protein Production with Ribosome Biogenesis

This example investigates the loss of 70S ribosome formation and an alteration in the mobility of the 50S subunit that is observed in YlqF protein-depleted cells.

Strain RB301 cells will be grown in the presence of 1 mM, 50 μM, 10 μM, 5 μM, and no IPTG. Cell lysates will be prepared and run on 10-25% sucrose gradients and ribosome profiles will be generated according to Example XIX. 70S ribosome levels will directly correlate with the growth rate of the cells. This supports a role for YlqF proteins as a limiting step in 70S formation. This limiting step may comprise translation initiation or subunit biogenesis. An observation that the shift in the size of 50S subunit is observed in all cultures that are affected in growth, then the effect is directly due to YlqF protein depletion. YlqF protein depletion supports a model in which ribosome biogenesis is affected by a rate-limiting step that requires YlqF protein, and that once YlqF protein performs its function, then translation initiation can proceed and 70S ribosomes can be formed.

Intermediate levels of growth of the RB301 strain may correlate with the level of IPTG in the medium suggesting that YlqF protein is controlling a rate-limiting step affecting bacterial cell growth. Analysis of ribosome profiles from RB301 cultures growing at different rates will determine if: a) 70S ribosome levels directly correlate with the amount of YlqF protein in the cell; and b) if the abnormal migration of 50S subunit is still observed at faster growth rates. If abnormal 50S subunits are observed under all conditions tested, the results would support a model in which decreased expression of YlqF protein is limiting for translation by some effect on 50S biogenesis.

### EXAMPLE XXI

### Interaction of the YlqF Protein with the Ribosome

This example investigates the structural mechanics of YlqF protein interaction with the ribosome. Identifying the ribosomal component(s) to which a YlqF protein binds will aid in determining the function of YlqF protein.

Initially, it will be determined if YlqF proteins co-fractionate with ribosomes. The experiments will be done under conditions that have been shown to facilitate association of other bacterial GTPases with the ribosome. Daigle et al., “Studies of the interaction of Escherichia coli YjeQ with the ribosome in vitro” J Bacteriol 186:1381-7 (2004); Lin et al., “The Caulobacter crescentus CgtA(C) Protein Cosediments with the Free 50S Ribosomal Subunit” J Bacteriol 186:481-9 (2004); and Wout et al., “The Escherichia coli GTPase CgtAE cofractionates with the 50S ribosomal subunit and interacts with SpoT, a ppGpp synthetase/hydrolase” J Bacteriol 186:5249-57 (2004). The first experiment will determine if YlqF proteins remain with the ribosome in the S100 centrifugation pellet. Next, various salt and ion concentrations are tested to determine the stability of the interaction with the ribosome. After that, sucrose gradient density centrifugations will be performed on wild-type cells to further verify that YlqF proteins are associated with a specific ribosomal subunit.

Sucrose density fractions will be collected that contain polysomes, 70S ribosomes, 50S subunits, and the 30S subunits. Polyclonal antibodies to YlqF proteins will be incubated with Western blot electrophoretic gels directed against the isolated fractions. This technique will determine if YlqF protein co-fractionates with 70S ribosomes, 50S subunits, or 30S subunits. Alternatively, a composition comprising a YlqF -green fluorescent protein (GFP) fusion protein and GFP antibodies can be used to monitor the migration of YlqF proteins. A YlqF -GFP fusion bacterial strain has been verified as in which the fusion protein is the only YlqF protein being expressed in a viable cell and also supports wild-type growth (supra).

Alternatively, in vitro experiments may be performed to determine if YlqF protein associates with the ribosome. For example, purified YlqF -His₆ protein will be incubated with total cell lysates of YlqF protein-depleted cells. Lysates will be subjected to sucrose gradient density centrifugation, and individual fractions along the gradient will be isolated. The presence of YlqF-His₆ protein can be monitored by polyclonal YlqF protein antibodies (if available) or commercially available anti-His antibodies. Incubation conditions are determined empirically. A similar approach has been used in analyzing association of the essential GTPase Era protein with the ribosome in E. coli. Sayed et al., “Era, an essential Escherichia coli small G protein, binds to the 30S ribosomal subunit” Biochem Biophys Res Commun 264:51-4 (1999).

Alternatively, a cell biological approach may be used to monitor the presence of YlqF protein. For example, cell culture protein localization using proteins fused to GFP has been used to successfully gain information about the function of proteins involved in many processes including cell division, transcription and translation, and DNA replication. Lemon et al., “Localization of bacterial DNA polymerase: evidence for a factory model of replication” Science 282:1516-9 (1998); Lewis et al., “Compartmentalization of transcription and translation in Bacillus subtilis” Embo J 19:710-8 (2000); and Shapiro et al., “Protein localization and cell fate in bacteria” Science 276:712-8 (1997).

I claim:

1. A method to identify an antimicrobial compound, comprising:
a) providing:
i) a test compound;
ii) a first protein, said first protein comprising a GTPase;
iii) a second protein capable of interacting with said first protein, wherein said second protein comprises a ribosomal protein;
b) mixing said first and second protein in the presence of said test compound; and
c) measuring the interaction of said first and second proteins.

2. The method of claim 1, wherein said GTPase is selected from the group consisting of YlqF, YsxC, and YphC.

3. The method of claim 1, wherein said ribosomal protein is selected from the group consisting of L16, L35, and L36.

4. The method of claim 1, wherein said second protein comprises at least one fluorophore.

5. The method of claim 1, wherein said measuring comprises energy emission detection.

6. The method of claim 1, wherein said test compound comprises a protein translation inhibitor.

7. The method of claim 6, wherein said protein translation inhibitor binds to said GTPase.

8. The method of claim 7, wherein said binding prevents said interaction between said GTPase and said ribosomal protein.

9. The method of claim 6, wherein said protein translation inhibitor is selected from the group consisting of a polypeptide and a small molecular weight organic molecule.

10. A composition comprising a GTPase, a ribosomal protein, a ribosomal RNA, and a test compound, wherein said GTPase comprises a basic N-terminus and an acidic C-terminus.

11. The composition of claim 10, wherein said ribosomal protein interacts with said C-terminus.

12. The composition of claim 10, wherein said ribosomal RNA interacts with said N-terminus.

13. The composition of claim 10, wherein said test compound binds with said GTPase.

14. The composition of claim 10, wherein said GTPase is selected from the group consisting of YlqF, YqeH, YsxC, and YphC.

15. The composition of claim 10, wherein said ribosomal protein is selected from the group consisting of L16, L35, and L36.

16. The composition of claim 13, wherein said test compound comprises a protein translation inhibitor.

17. The method of claim 13, wherein said binding prevents said interaction between said GTPase and said ribosomal protein.

18. The method of claim 16, wherein said protein translation inhibitor is selected from the group consisting of a polypeptide and a small molecular weight organic molecule.

19. The method of claim 10, wherein said ribosomal RNA is selected from the group consisting of 5S, 16S, and 23S rRNA.
